(19) United States
(12) Patent Application Publication
(10) Pub. No.: US 2023/0242989 A1
(43) Pub. Date: Aug. 3, 2023

(54) TAGMENTATION TO OPEN UP CIRCLES OF DNA AND DETECT EXTRACHROMOSOMAL CIRCLES OF DNA FOR DIAGNOSIS

(71) Applicant: University of Virginia Patent Foundation, Charlottesville, VA (US)

(72) Inventors: Anindya Dutta, Charlottesville, VA (US); Pankaj Kumar, Crozet, VA (US)

(21) Appl. No.: 17/603,150

(22) PCT Filed: Apr. 13, 2020
(86) PCT No.: PCT/US2020/027938

§ 371 Date: Oct. 12, 2021

Related U.S. Application Data
(60) Provisional application No. 62/832,443, filed on Apr. 11, 2019.

(51) Int. Cl. C12Q 1/6886; C12N 15/10

(52) U.S. Cl. CPC C12Q 1/6886; C12N 15/1065; C12Q 1/6806

(57) ABSTRACT

Provided are methods and kits for detecting an extrachromosomal circular DNA (eccDNA) in a biological sample. In some embodiments, the method comprises treating the biological sample to produce a tagged linearized fragment of genomic DNA; and determining whether the tagged linearized fragment is derived from an eccDNA to thereby detect the eccDNA. In some embodiments, the treating of the biological sample to produce a tagged linearized fragment of genomic DNA comprises treating the biological sample with an insertional enzyme complex to produce a tagged linearized fragment of genomic DNA.

### CROSS REFERENCE TO RELATED APPLICATION

The presently disclosed subject matter claims the benefit of U.S. Provisional Pat. Application Serial Nos. 62/832,443, filed Apr. 11, 2019; the disclosure of which is incorporated herein by reference in its entirety.

### GOVERNMENT INTEREST

This invention was made with government support under Grant No. CA060499 awarded by The National Institutes of Health. The government has certain rights in the invention.

### BACKGROUND

The presence of tens of thousands of extrachomosomal DNA (eccDNA) in the nuclei of human and mouse cell lines as well as normal tissues and cancers has been previously reported (Dillon et al., Cell Rep 11, 1749-1759 (2015); Kumar et al., Mol Cancer Res 15, 1197-1205 (2017); Shibata et al., Science 336, 82-86 (2012)). Several other groups have also described the presence of eccDNAs in various eukaryotes ranging from yeasts to humans (Moller et al., G3 (Bethesda) 6, 453-462 (2015); Moller et al., Proc Natl Acad Sci USA 112, E3114-3122 (2015); Moller et al., Nat Commun 9, 1069 (2018); deCarvalho et al., Nat Genet 50, 708-717 (2018); Shoura et al., G3 (Bethesda) 7, 3295-3303 (2017); Turner et al., Nature 543, 122-125 (2017)). More recently it has been shown that circular DNA promotes the expression of oncogenes (Wu et al., Nature 575, 699-703 (2019)). Not only the oncogenes but also the regulatory regions associated with genes are also amplified as eccDNA (Morton et al., Cell 179, 1330-1341 e1313 (2019)). Thus, approaches are needed in the art to assess and characterize eccDNAs from samples.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

A method of detecting an extrachromosomal circular DNA (eccDNA) in a biological sample is provided in accordance with the presently disclosed subject matter. In some embodiments, the method comprises treating the biological sample to produce a tagged linearized fragment of genomic DNA; and determining whether the tagged linearized fragment is derived from an eccDNA to thereby detect the eccDNA. In some embodiments, the treating of the biological sample to produce a tagged linearized fragment of genomic DNA comprises treating the biological sample with an insertional enzyme complex to produce a tagged linearized fragment of genomic DNA.

In some embodiments, determining whether the tagged linearized fragment is derived from an eccDNA comprises amplifying the tagged linearized fragment of genomic DNA. In some embodiments, determining whether the tagged linearized fragment is derived from an eccDNA comprises sequencing the tagged linearized fragment of genomic DNA. In some embodiments, determining whether the tagged linearized fragment is derived from an eccDNA comprises detecting a junctional sequence in the tagged linearized fragment. In some embodiments, detecting a junctional sequence comprises employing read pairs.

In some embodiments, the method further comprising treating the sample with an exonuclease prior to treating the biological sample to produce a tagged linearized fragment of genomic DNA, optionally treating the sample with an exonuclease prior to treating the biological sample with an insertional enzyme complex to produce a tagged linearized fragment enriched from eccDNA. In some embodiments, the insertional enzyme complex comprises an insertional enzyme and at least two adaptors molecules. In some embodiments, the insertional enzyme is a transposase. In some embodiments, the transposase is a Tn5 transposase.

In some embodiments, the sample comprises a biopsy or a blood sample. In some embodiments, the subject is a subject suffering from a cancer or suspected to be suffering from a cancer or a subject having a genetic disease or disorder or suspected to have a genetic disease or disorder. In some embodiments, the subject having a genetic disease or disorder or suspected to have a genetic disease or disorder is a fetus and the sample comprises a maternal blood sample.

In some embodiments, the presently disclosed subject matter provides for analyzing a sample from a subject to detect eccDNA; and providing a diagnosis or prognosis based on the detected eccDNA. In some embodiments, providing a diagnosis or prognosis comprises identifying a cell type in the subject, identifying a cell population, identifying a tissue type, and/or identifying a nucleic acid sequence on the eccDNA. In some embodiments, the method further comprises choosing a therapy based on the diagnosis or prognosis, optionally based on the identified cell type, cell population, tissue type, or nucleic acid.

In some embodiments, the presently disclosed subject matter provides a method of detecting a cell type, a population of cells, or a tissue type in a subject. In some embodiments, the method comprises (a) detecting an extrachromosomal circular DNA (eccDNA) in a biological sample from the subject by: (i) treating the biological sample to produce a tagged linearized fragment of genomic DNA; and (ii) determining whether the tagged linearized fragment is derived from an eccDNA to thereby detect the eccDNA; and (b) determining a genomic region from which the eccDNA is derived to thereby detect a cell type, a population of cells, or a tissue type in a subject.

In some embodiments, treating the biological sample to produce a tagged linearized fragment, optionally enriched from genomic eccDNA, comprises treating the biological sample with an insertional enzyme complex to produce a tagged linearized fragment of genomic DNA, optionally treating the biological sample with an exonuclease to digest genomic linear DNA and then with an insertional enzyme complex to produce a tagged linearized fragment of genomic DNA. In some embodiments, determining whether the tagged linearized fragment is derived from an eccDNA comprises amplifying the tagged linearized fragment of genomic DNA. In some embodiments, determining whether the tagged linearized fragment is derived from an eccDNA comprises sequencing the tagged linearized fragment of genomic DNA. In some embodiments, determining whether the tagged linearized fragment is derived from an eccDNA comprises detecting a junctional sequence in the tagged linearized fragment. In some embodiments, detecting a junctional sequence comprises employing read pairs.

In some embodiments, the method further comprises treating the sample with an exonuclease prior to treating the biological sample to produce a tagged linearized fragment of genomic DNA. In some embodiments, the insertional enzyme complex comprises an insertional enzyme and at least two adaptors molecules. In some embodiments, the insertional enzyme is a transposase. In some embodiments, the transposase is a Tn5 transposase.

In some embodiments, the sample comprises a biopsy or a blood sample. In some embodiments, the subject is a subject suffering from a cancer or suspected to be suffering from a cancer or a subject having a genetic disease or disorder or suspected to have a genetic disease or disorder. In some embodiments, the subject having a genetic disease or disorder or suspected to have a genetic disease or disorder is a fetus and the sample comprises a maternal blood sample.

In some embodiments, identifying a cell type in the subject, identifying a cell population, identifying a tissue type; and/or identifying a nucleic acid sequence on the eccDNA further comprises identifying a cancer or a genetic disease or disorder in the subject. In some embodiments, the method further comprises choosing a therapy based on the identified cell type, cell population, tissue type, and/or nucleic acid sequence.

In some embodiments, the presently disclosed subject matter provides a method of detecting a nucleic acid sequence associated with a condition in a subject. In some embodiments, the method comprises (a) detecting an extrachromosomal circular DNA (eccDNA) in a sample from the subject by: (i) treating the biological sample to produce a tagged linearized fragment of genomic DNA; and (ii) determining whether the tagged linearized fragment is derived from an eccDNA to thereby detect the eccDNA; and (b) detecting a presence of a nucleic acid sequence on the eccDNA, wherein the nucleic acid sequence is associated with a condition in the subject.

In some embodiments, treating the biological sample to produce a tagged linearized fragment, optionally enriched from genomic eccDNA, comprises treating the biological sample with an insertional enzyme complex to produce a tagged linearized fragment of genomic DNA, optionally treating the biological sample with an exonuclease to digest genomic linear DNA and then with an insertional enzyme complex to produce a tagged linearized fragment of genomic DNA. In some embodiments, determining whether the tagged linearized fragment is derived from an eccDNA comprises amplifying the tagged linearized fragment of genomic DNA. In some embodiments, determining whether the tagged linearized fragment is derived from an eccDNA comprises sequencing the tagged linearized fragment of genomic DNA. In some embodiments, determining whether the tagged linearized fragment is derived from an eccDNA comprises detecting a junctional sequence in the tagged linearized fragment. In some embodiments, detecting a junctional sequence comprises employing read pairs.

In some embodiments, the method further comprises treating the sample with an exonuclease prior to treating the biological sample to produce a tagged linearized fragment of genomic DNA. In some embodiments, the insertional enzyme complex comprises an insertional enzyme and at least two adaptors molecules. In some embodiments, the insertional enzyme is a transposase. In some embodiments, the transposase is a Tn5 transposase.

In some embodiments, the sample comprises a biopsy or a blood sample. In some embodiments, the subject is a subject suffering from a cancer or suspected to be suffering from a cancer or a subject having a genetic disease or disorder or suspected to have a genetic disease or disorder. In some embodiments, the subject having a genetic disease or disorder or suspected to have a genetic disease or disorder is a fetus and the sample comprises a maternal blood sample.

In some embodiments, identifying a cell type in the subject, identifying a cell population, identifying a tissue type; and/or identifying a nucleic acid sequence on the eccDNA further comprises identifying a cancer or a genetic disease or disorder in the subject. In some embodiments, the method further comprises choosing a therapy based on the identified cell type, cell population, tissue type, and/or nucleic acid sequence.

In some embodiments, a kit for detecting eccDNA in a sample is disclosed. In some embodiments, the kit comprises one or more reagents suitable for carrying out a method in accordance with the presently disclosed subject matter, and instructional material for employing the one or more reagents.

Accordingly, it is an object of the presently disclosed subject matter to provide methods for detecting eccDNA in a sample. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A to 1C show that a circle could be part of an ATAC-seq library. FIG. 1A is a schematic showing that if circular DNA has open chromatin structure near or around the ligation point the library preparation method will cut and attach an adaptor into a DNA fragment from eccDNA. FIG. 1B is a schematic one end of paired end read mapping on the body of a circular DNA with read from the other end mapping on the ligation junction. FIG. 1C is a flow chart showing detailed steps from mapping to identification of the new Circle_finder pipeline.

FIG. 2 is a plot showing length distribution of identified eccDNA in C4-2 and OVCAR8 cell lines.

FIGS. 3A to 3D show experimental validation of randomly selected eccDNA identified by ATAC-seq in C4-2B and OVCAR8 cells. FIG. 3A is a schematic for isolation and detection of extra-chromosomal circular DNA. See Materials and Methods for EXAMPLES 1-8 for details. FIG. 3B is a digital image of an electrophoresis gel showing PCR detection of eccDNA. DNA bands marked with boxes were gel purified and sequenced. FIG. 3C is a table describing eccDNAs validated in FIG. 3B, based on analysis of ATAC-seq data from OVCAR8 and C4-2B. FIG. 3D is a schematic showing junctional tags obtained after sequencing of PCR products in FIG. 3B. Shaded and unshaded sequences depict 15 bases on either side of junctions (SEQ ID NOs: 1-11 from top to bottom, respectively; see also Table 2 herein below). Numbers indicate chromosomal location on respective chromosomes C1-C11, sequences provided in the Sequence Listing as follows: C1, SEQ ID NO: 12; C2, SEQ ID NO: 13; C3, SEQ ID NO: 14; C4, SEQ ID NO: 15; C5, SEQ ID NO: 16; C6, SEQ ID NO: 17; C7, SEQ ID NO: 18; C8, SEQ ID NO: 19; C9, SEQ ID NO: 20; C10, SEQ ID NO: 21; C11, SEQ ID NO: 22. Note the match between numbers for each circle in FIG. 3C and FIG. 3D. Some of the junction sequence identified by Sanger sequencing differ by few bases due to multiple species of eccDNA present in given cell lines. Oval circles represent insertion and boxed sequences represent mismatches. *-Sequence obtained from the bottom strand.

FIGS. 4A to 4E show eccDNA in cell lines and LGG or GBM tumors. FIG. 4A is a set of digital images showing detection of EccDNA in OVCAR8 cell line by FISH: Metaphase spread of chromosome (larger lighter areas; blue when seen in color) from OVCAR8 cells were stained with the probe (smaller lighter areas; green when seen in color) against the eccDNA locus chr2: 238136071-238170279 (Top Row) or chr10: 103457331-103528085 (Bottom Row). The spreads on the left do not have an extrachromosomal signal, while the spreads on the right have extrachromosomal signals that are better seen in the magnified insets on the extreme right. White arrowheads mark the eccDNA signals. FIG. 4B is a digital imaging showing that for the negative control cell lines, C4-2, the spread does not have extrachromosomal DNA of the type being probed for. FIG. 4C is a plot showing that the eccDNA signals in OVCAR8 (n = 28) and C4-2 (n = 24) (negative control) were quantified for locus chr10: 103457331-103528085 and shown in the graph. P values were calculated using student’s t-test, **p<0.01. FIG. 4D is a plot showing that eccDNA/duplication loci identified in WGS libraries show genomic amplification (median 1.5 fold), suggesting at least one allele is duplicated in all the cells, but this would be difficult to call reliably because of statistical variations and would need the expense of whole genome sequencing. eccDNA loci identified in ATAC-seq libraries do not show genomic amplification (median close to zero). Thus, the eccDNA are apparent by the presently disclosed method before a copy number variation (CNV) can be reliably detected at the locus by genome sequencing methods. Value of CNA in Y-axis is in log2. FIG. 4E is a plot showing length distribution of eccDNA identified in LGG and GBM TCGA ATAC-seq data.

FIGS. 5A to 5E show properties of microDNA identified herein (eccDNA<1 kb) by ATAC-seq. FIG. 5A is a plot showing length distribution of eccDNA shows peaks at 180 and 380 bases. FIG. 5B is a plot showing that the GC content of eccDNA locus and regions immediately upstream and downstream from the eccDNA is higher than genomic average as calculated from 1000 random stretches of the genome of equivalent length as the eccDNA (Random-1000). FIG. 5C is a bar graph showing that the sites in the genome that give rise to small eccDNA are enriched relative to random expectation in genic sites, sequences 2 kb upstream from genes and in CpG islands. FIG. 5D is a bar graph showing that direct repeats of 2-15 bp flanking the genomic locus of the eccDNA at ligation point are present for about 20% of the loci. FIG. 5E is a heat map showing gene classes enriched in the set of genes found on the circular DNAs in two or more cancers. The shading scale (light to dark shading; blue to black when shown in color) indicates enrichment in pathway (lighter shading or blue color indicates pathway was enriched). If the genes found on the eccDNA/duplication loci in a cancer type are significantly enriched in the indicated pathways, the color in the cell is light gray (blue when shown in color). If the set of genes is not enriched in that cancer, the cell is black.

FIG. 6 is a plot showing length distribution of identified eccDNA in GBM cell lines.

FIG. 7 is a plot showing copy number analysis limitation with TCGA SNP array hybridization data. In general, genotyping arrays will fail to identify amplifications caused by the circular DNAs detected in the presently disclosed analysis. The minimum detectable segment length (y-axis) is partially dependent on the extent of amplification (x-axis). Assuming that one copy of a circular DNA is present in every cell of a patient’s tumor, the segment mean would be 0.585 and the minimum detectable length would therefore be approximately 1.5 MB. Given that the average length of the circular DNAs detected in this analysis was 2 kb, the circular DNAs detected in this analysis would not show up as amplifications when genotyping array data is analyzed.

FIG. 8 is a table providing example of circles identified in HCT116 ATAC-Seq data. Split reads and mapped read mapping position is given for each of the mapped-unmapped pairs.

FIG. 9 is a plot showing length distribution of circles identified in bulk HCT116 cell population by ATAC-Seq method.

FIG. 10 is a plot showing circles identified in single cell ATAC-seq data. Median number of microDNA per cell in various cell type.

FIG. 11 is a set of TSNE plots showing that microDNA are generated in a tissue specific manner. The TSNE plots are based on profiles of where in the genome the microDNA arise from. Left: MicroDNA profiles in human fibroblasts & lymphoblasts. Right: Mouse cardiomyocyte & CD4+ cells.

FIG. 12 is a schematic of tumor cells, showing an approach for detecting eccDNAs pre-amplification to avoid drugs to which the tumor will quickly become resistant by amplifying the drug-resistant gene.

FIG. 13 is a schematic showing eccDNA map will identify genes poised to amplify at the pre-amplification step. A blow-up of a section of a karyotype plot is shown on the left side of FIG. 13, while a blow-up of a section of a plot of copy number versus chromosome position from Zong, Science, 2012, is shown on the right side to illustrate how CNV is called by current genomic methods.

FIG. 14 is a plot of abundance versus eccDNAs length (kb) in cancer cells and in normal cells.

### REFERENCE TO SEQUENCE LISTING

The Sequence Listing associated with the instant disclosure has been submitted electronically herewith as an 801 kilobyte file with File Name (Sequence_Listing_3062-123_PCT_ST25.txt), Creation Date (Apr. 13, 2020), Computer System (IBM-PC/MS-DOS/MS-Windows), and Docket No. (3062/123 PCT). The Sequence Listing submitted electronically herewith is hereby incorporated by reference into the instant disclosure.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

ATAC-seq (Assay for Transposase-Accessible Chromatin using sequencing) identifies open chromatin regions all across the genome (Buenrostro et al., Curr Protoc Mol Biol 109, 21 29 21-29 (2015)). The method uses the hyperactive transposase Tn5 to cut the accessible chromatin with simultaneous ligation of adapters at cut sites (Buenrostro et al., Curr Protoc Mol Biol 109, 21 29 21-29 (2015)). Since isolated nuclei as a whole are subjected to the transposition reaction in ATAC-seq, the presently disclosed subject matter relates to the investigation of whether the transposase will also cleave DNA from eccDNAs, and so the ATAC-seq libraries will contain fragments of DNA from eccDNA. Thus, the presently disclosed subject matter relates in some embodiments to the use of transposase (tagmentation) to linearize circles of extrachromosomal DNA and high throughput sequencing to identify such circles in cells, cancers, and body fluids. Particularly, it is demonstrated herein that opening the circle by transposase (tagmentation) detects circles of DNA very efficiently. ATAC-seq data was analyzed from cell populations and from single cells where the transposase was used to fragment cellular DNA, which was subsequently sequenced. The presently disclosed subject matter thus provides in some embodiments that transposase tagging of DNA followed by high throughput sequencing efficiently identifies circles of DNA.

Continuing, extrachromosomal circular DNAs (eccDNAs) are usually somatically mosaic and a source of intercellular heterogeneity in normal and tumor cells. Because short eccDNAs are poorly chromatinized, in accordance with aspects of the presently disclosed subject matter they were sequenced by tagmentation in ATAC-seq experiments, without any enrichment of circular DNA. Thousands of eccDNAs were identified. The eccDNAs identified in cell lines were validated by inverse PCR on DNA that survives exonuclease digestion of linear DNA, and by metaphase FISH. ATAC-seq in gliomas and glioblastomas identify hundreds of eccDNAs, including one containing the well-known EGFR gene amplicon from chr7. Over 18,000 eccDNAs, many carrying known cancer driver genes, are identified in a pan-cancer analysis of 360 ATAC-seq libraries from 23 tumor types. Because of somatic mosaicism, eccDNAs are identified by ATAC-seq even before amplification of the locus is recognized by genome-wide copy number variation measurements. Thus, standard ATAC-seq is a sensitive method to detect eccDNA present in a subset of tumor cells, ready to be amplified under appropriate selection, as during therapy.

According to exemplary embodiments of the presently disclosed subject matter, ATAC-seq libraries were first preparing using C4-2B (prostate cancer) and OVCAR8 (ovarian cancer) cell lines. Hundreds of eccDNAs were identified using the presently disclosed computational pipeline. Inverse PCR on exonuclease resistant extrachromosmal DNA (highly enriched in circular DNA) and FISH on metaphase spreads confirmed the presence of the identified somatically mosaic eccDNA. To provide additional evidence of the success of ATAC-seq in identifying eccDNA, an ATAC-seq library generated from patient-derived GBM cell lines (Xie et al., Cell 175, 1228-1243 e1220 (2018)) was analyzed and the eccDNA harboring EGFR gene was identified, which is known to be amplified through the formation of eccDNA in GBM. Finally, ATAC-seq data from GBM and LGG generated by the TCGA consortium was analyzed to identify hundreds of eccDNAs even before their amplification was apparent as a copy number variation by hybridization to SNP arrays. Genes involved in pathways related to nucleosomal events were significantly enriched in these loci.

Headings are included herein for reference and to aid in locating certain sections. These headings are not intended to limit the scope of the concepts described therein under, and these concepts may have applicability in other sections throughout the entire specification.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, amino acids are represented by the full name thereof, by the three letter code corresponding thereto, or by the one-letter code corresponding thereto, as indicated in Table 1:

TABLE 1

Amino Acid Codes and Functionally Equivalent Codons

| | | | |
|---|---|---|---|
| Full Name | 3-Letter Code | 1-Letter Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide’s circulating half-life without adversely affecting their activity. Additionally, a disulfide linkage may be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

Amino acids have the following general structure:

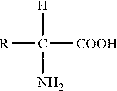

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “compound”, as used herein, refers to any type of substance or agent that is commonly considered a drug, or a candidate for use as a drug, combinations, and mixtures of the above, as well as other non-limiting examples like polypeptides and antibodies.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal’s health continues to deteriorate.

In contrast, a “disorder” in an animal is a state of health in which the animal is able to maintain homeostasis, but in which the animal’s state of health is less favorable than it would be in the absence of the disorder. Left untreated, a disorder does not necessarily cause a further decrease in the animal’s state of health.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5’-ATTGCC-3’ and 5’-TATGGC-3’ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs (see Altschul et al., 1990a; Altschul et al., 1990b), and can be accessed, for example at the National Center for Biotechnology Information (NCBI) world wide web site. BLAST nucleotide searches can be performed with the NBLAST program (designated “blastn” at the NCBI web site), using the following parameters: gap penalty = 5; gap extension penalty = 2; mismatch penalty = 3; match reward = 1; expectation value 10.0; and word size = 11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

Used interchangeably herein are the terms “isolate” and “select”.

The term “isolated”, when used in reference to cells, refers to a single cell of interest, or population of cells of interest, at least partially isolated from other cell types or other cellular material with which it naturally occurs in the tissue of origin. A sample of stem cells is “substantially pure” when it is in some embodiments at least 60%, in some embodiments at least 75%, in some embodiments at least 90%, and, in certain cases, in some embodiments at least 99% free of cells other than cells of interest. Purity can be measured by any appropriate method, for example, by fluorescence-activated cell sorting (FACS), or other assays, which distinguish cell types.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to either a molecule that joins two other molecules covalently or noncovalently, e.g., through ionic or hydrogen bonds or van der Waals interactions.

The terms “gene product” or “expression product” are used herein interchangeably to refer to the RNA transcription products (RNA transcript) of a gene, including mRNA, and the polypeptide translation product of such RNA transcripts. A gene product may be, for example, a polynucleotide gene expression product (e.g., an unspliced RNA, an mRNA, a splice variant mRNA, a microRNA, a fragmented RNA, and the like) or a protein expression product (e.g., a mature polypeptide, a post-translationally modified polypeptide, a splice variant polypeptide, and the like). In some embodiments the gene expression product may be a sequence variant including mutations, fusions, loss of heterozygoxity (LOH), and/or biological pathway effects.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature that may be used. As a result, it follows that higher relative temperatures may tend to make the reaction conditions more stringent, while lower temperatures less so. For additional details and explanation of stringency of hybridization reactions, see Ausubel et al., 1995.

“Stringent conditions” or “high stringency conditions”, as defined herein, typically: (1) employ low ionic strength solutions and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 0.075 M sodium citrate), 50 mM sodium phosphate (pH 6.8), 0.1% sodium pyrophosphate, 5× Denhardt’s solution, sonicated salmon sperm DNA (50 µg/ml), 0.1% SDS, and 10% dextran sulfate at 42° C., with washes at 42° C. in 0.2× SSC (sodium chloride/sodium citrate) and 50% formamide at 55° C., followed by a high-stringency wash consisting of 0.1× SSC containing EDTA at 55° C.

“Moderately stringent conditions” may be identified as described by Sambrook et al., 1989, and include the use of washing solution and hybridization conditions (e.g., temperature, ionic strength and % SDS) less stringent that those described above. An example of moderately stringent condition is overnight incubation at 37° C. in a solution comprising: 20% formamide, 5×SSC (150 mM NaCl, 15 mM trisodium citrate), 50 mM sodium phosphate (pH 7.6), 5×Denhardt’s solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

“Sensitivity” as used herein refers to the proportion of true positives of the total number tested that actually have the target disorder (i.e., the proportion of patients with the target disorder who have a positive test result). “Specificity” as used herein refers to the proportion of true negatives of all the patients tested who actually do not have the target disorder (i.e., the proportion of patients without the target disorder who have a negative test result).

In the context of the present disclosure, reference to “at least one,” “at least two,” “at least five,” etc. of the genes listed in any particular gene set means any one or any and all combinations of the genes listed.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5ʹ-end; the left-hand direction of a double-stranded polynucleotide sequence is referred to as the 5ʹ-direction. The direction of 5ʹ to 3ʹ addition of nucleotides to nascent RNA transcripts is referred to as the transcription direction. The DNA strand having the same sequence as an mRNA is referred to as the “coding strand”; sequences on the DNA strand which are located 5’ to a reference point on the DNA are referred to as “upstream sequences”; sequences on the DNA strand which are 3ʹ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest, including, but not limited to, wound healing, angiogenesis, bone healing, osteoblast production and function, and osteoclast production, differentiation, and activity.

The term “subject,” as used herein, generally refers to a mammal. Typically, the subject is a human. However, the term embraces other species, e.g., pigs, mice, rats, dogs, cats, or other primates. In certain embodiments, the subject is an experimental subject such as a mouse or rat. The subject may be a male or female. The subject may be an infant, a toddler, a child, a young adult, an adult or a geriatric. The subject may exhibit one or more symptoms of IPF. For example, the subject may exhibit shortness of breath (generally aggravated by exertion) and/or dry cough), and, in some cases may have obtained results of one or more of an imaging test (e.g., chest X-ray, computerized tomography (CT)), a pulmonary function test (e.g., spirometry, oximetry, exercise stress test), lung tissue analysis (e.g., histological and/or cytological analysis of samples obtained by bronchoscopy, bronchoalveolar lavage, surgical biopsy) that is indicative of the potential presence of IPF. A subject under the care of a physician or other health care provider may be referred to as a “patient”.

A “subject” of diagnosis or treatment is an animal, including a human. It also includes pets and livestock.

As used herein, a “subject in need thereo” is a patient, animal, mammal, or human, who will benefit from the method of the presently disclosed subject matter.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14. algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2X standard saline citrate (SSC), 0.1% SDS at 50° C.; in some embodiments in 7% (SDS), 0.5 M NaPO4, 1 mM EDTA at 50° C. with washing in 1X SSC, 0.1% SDS at 50° C.; in some embodiments 7% SDS, 0.5 M NaPO4, 1 mM EDTA at 50° C. with washing in 0.5X SSC, 0.1% SDS at 50° C.; and more in some embodiments in 7% SDS, 0.5 M NaPO4, 1 mM EDTA at 50° C. with washing in 0.1X SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### II. Exemplary Embodiments

A method of detecting an extrachromosomal circular DNA (eccDNA) in a biological sample is provided in accordance with the presently disclosed subject matter. In some embodiments, the method comprises treating the biological sample to produce a tagged linearized fragment of genomic DNA; and determining whether the tagged linearized fragment is derived from an eccDNA to thereby detect the eccDNA. In some embodiments, the genomic DNA comprises accessible chromatin or whole genome or exonuclease resistant DNA from the cells. In some embodiments, the biological sample is treated with a restriction enzyme and ligated to sequencing primers to produce a tagged linearized fragment of genomic DNA. In some embodiments, the fragments are sequenced by high throughput sequencing to identify the junctional sequence that indicates the presence of an eccDNA.

Any suitable restriction enzyme (also referred to as restriction endonucleases) and suitable reaction conditions and reagents as would be apparent to one of ordinary skill in the art upon a review on the instant disclosure can be employed. Restriction endonucleases are available from many commercial sources, such as Thermo Fisher Scientific and Sigma Aldrich. By way of example and not limitation, Type I, Type II, Type III, and/or Type IV restriction enzymes can be employed. Additional specific no limiting examples include Asc1, EcoR1, HindIII, and/or XhoI restriction enzymes can be employed.

Ligation can be accomplished either enzymatically or chemically. “Ligation” means to form a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between 5ʹ carbon of a terminal nucleotide of the tagged fragment of genomic DNA with the 3ʹ carbon of the tagged fragment of genomic DNA.

A variety of template-driven ligation reactions are described in the following references: Whitely et al., U.S. Pat. No. 4,883,750; Letsinger et al., U.S. Pat. No. 5,476,930; Fung et al., U.S. Pat. No. 5,593,826; Kool, U.S. Pat. No. 5,426,180; Landegren et al., U.S. Pat. No. 5,871,921; Xu and Kool (1999) Nucl. Acids Res. 27:875; Higgins et al., Meth. in Enzymol. (1979) 68:50; Engler et al. (1982) The Enzymes, 15:3 (1982); and Namsaraev, U.S. Pat. Pub. 2004/0110213.

Chemical ligation methods are disclosed in Ferris et al., Nucleosides & Nucleotides, 8: 407-414 (1989) and Shabarova et al., Nucleic Acids research, 19: 4247-4251 (1991). Enzymatic ligation utilizes a ligase. Many ligases are known to those of skill in the art as referenced in Lehman, Science, 186: 790-797 (1974); Engler et al., DNA ligases, pages 3-30 in Boyer, editor, The Enzymes, Vol. 15B (Academic Press, New York, 1982); and the like. Exemplary ligases include SplintR ligase, T4 DNA ligase, T7 DNA ligase, E.coli DNA ligase, Taq ligase, Pfu ligase and the like. Certain protocols for using ligases are disclosed by the manufacturer and also in Sambrook, Molecular Cloning: A Laboratory manual, 2^{nd} Edition (Cold Spring Harbor Laboratory, New York, 1989); Barany, PCR Methods and Applications, 1:5-16 (1991); Marsh et al., Strategies, 5:73-76 (1992). In one embodiment, the ligase may be derived from algal viruses such as the Chlorella virus, for example, PBCV-1 ligase, also known as SplintR ligase, as described U.S. Pat. Publication No. 2014/0179539, incorporated herein by reference in its entirety.

In some embodiments, the treating of the biological sample to produce a tagged linearized fragment of genomic DNA comprises treating the biological sample with an insertional enzyme complex to produce a tagged linearized fragment of genomic DNA. In some embodiments, the method comprises treating the sample with an exonuclease prior to treating the biological sample with an insertional enzyme complex to produce a tagged linearized fragment that is enriched from eccDNA. In some embodiments, the method comprises treating the sample with an exonuclease prior to treating the biological sample with restriction enzyme and ligation of sequencing primers to produce a tagged linearized fragment that is enriched from eccDNA.

In some embodiments, the presently disclosed subject matter provides for analyzing a sample from a subject to detect eccDNA; and providing a diagnosis or prognosis based on the detected eccDNA. In some embodiments, providing a diagnosis or prognosis comprises identifying a cell type in the subject, identifying a cell population, identifying a tissue type; and/or identifying a nucleic acid sequence on the eccDNA. In some embodiments, the method further comprises choosing a therapy based on the diagnosis or prognosis, optionally based on the identified cell type, cell population, tissue type, or nucleic acid. In some embodiments, this approach is used to monitor a therapeutic treatment in a subject. In some embodiments the method comprises administering the therapy to the subject. Representative non-limiting genes for analysis are provided in the Examples.

In some embodiments, the presently disclosed subject matter provides a method of detecting a cell type, a population of cells, or a tissue type in a subject. In some embodiments, the method comprises (a) detecting an extrachromosomal circular DNA (eccDNA) in a biological sample from the subject by: (i) treating the biological sample to produce a tagged linearized fragment of genomic DNA; and (ii) determining whether the tagged linearized fragment is derived from an eccDNA to thereby detect the eccDNA; and (b) determining a genomic region from which the eccDNA is derived to thereby detect a population of cells or a tissue type in a subject. In some embodiments, treating the biological sample to produce a tagged linearized fragment of genomic eccDNA comprises treating the biological sample with an exonuclease to digest genomic linear DNA and then with an insertional enzyme complex to produce a tagged linearized fragment of genomic DNA. In some embodiments, this approach is used to monitor a therapeutic treatment in a subject and/or to choose a therapy for the subject. In some embodiments the method comprises administering the therapy to the subject. Representative non limiting genes for analysis are disclosed in the Examples.

In some embodiments, the presently disclosed subject matter provides a method of detecting a nucleic acid sequence associated with a condition in a subject. In some embodiments, the method comprises (a) detecting an extrachromosomal circular DNA (eccDNA) in a sample from the subject by: (i) treating the biological sample to produce a tagged linearized fragment of genomic DNA; and (ii) determining whether the tagged linearized fragment is derived from an eccDNA to thereby detect the eccDNA; and (b) detecting a presence of a nucleic acid sequence on the eccDNA, wherein the nucleic acid sequence is associated with a condition in the subject. In some embodiments, treating the biological sample to produce a tagged linearized fragment of genomic eccDNA comprises treating the biological sample with an exonuclease to digest genomic linear DNA and then with an insertional enzyme complex to produce a tagged linearized fragment of genomic DNA. In some embodiments, this approach is used to monitor a therapeutic treatment in the subject. In some embodiments the method comprises administering the therapy to the subject. Representative non-limiting genes for analysis are disclosed in the EXAMPLES. In some embodiments, the condition comprises a disease or disorder as described herein

The term “insertional enzyme complex,” as used herein, refers to a complex comprising an insertional enzyme and two adaptor molecules (also referred to as the “molecular tags” or “transposon tags”) that are combined with polynucleotides to fragment and add adaptors to the polynucleotides. Such a system is described in a variety of publications, including Caruccio (Methods Mol. Biol. 2011 733: 241-55), US20100120098 and US20160060691, which are incorporated by reference herein. The insertional enzyme can be a transposase. In some embodiments, the transposase can be derived from Tn5 transposase. In other embodiments, the transposase can be derived from MuA transposase. In further embodiments, the transposase can be derived from Vibhar transposase (e.g. from Vibrioharveyi). In some embodiments, the insertional enzyme can comprise two or more enzymatic moieties wherein each of the enzymatic moieties inserts a common sequence into the genomic DNA. The enzymatic moieties can be linked together. The common sequence can comprise a common barcode. The enzymatic moieties can comprise transposases. The genomic DNA can be fragmented into a plurality of fragments.

The term “insertional enzyme complex,” as used herein, refers to a complex comprising an insertional enzyme and at least two adaptor molecules (the “transposon tags”) that are combined with polynucleotides to fragment and add adaptors to the polynucleotides. In some embodiments, the genomic DNA can be fragmented into a plurality of fragments during the insertion of the molecular tags. In this step, the genomic DNA is tagmented (i.e., cleaved and tagged in the same reaction) using an insertional enzyme such as a transposase that cleaves the genomic DNA in open regions in the chromatin and adds adaptors to both ends of the fragments. Methods for tagmenting isolated genomic DNA are known in the art (see, e.g., Caruccio Methods Mol. Biol. 2011 733: 241-55; Kaper et al, Proc. Natl. Acad. Sci. 2013 110: 5552-7; Marine et al, Appl. Environ. Microbiol. 2011 77: 8071-9, US20100120098, US20160060691, US2019/0032128) and are commercially available from Illumina (San Diego, Calif.) and other vendors. Such systems may be readily adapted for use herein. In some cases, the conditions may be adjusted to obtain a desirable level of insertion in the genomic DNA (e.g., an insertion that occurs, on average, every 50 to 200 base pairs in open regions). Other approaches are disclosed in the EXAMPLES set forth herein below.

The insertional enzyme can be any enzyme capable of inserting a nucleic acid sequence into a polynucleotide. In some cases, the insertional enzyme can insert the nucleic acid sequence into the polynucleotide in a substantially sequence-independent manner. The insertional enzyme can be prokaryotic or eukaryotic. Examples of insertional enzymes include, but are not limited to, transposases, HERMES, and HIV integrase. The transposase can be a Tn transposase (e.g., Tn3, Tn5, Tn7, Tn10, Tn552, Tn903), a MuA transposase, a Vibhar transposase (e.g., from Vibrioharveyi), Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Te1, THE-1, Tn/O, TnA, Tn3, Tn5, Tn7, Tn10, Tn552, Tn903, Toll, Tol2, Tn1O, Ty1, any prokaryotic transposase, or any transposase related to and/or derived from those listed above. In certain instances, a transposase related to and/or derived from a parent transposase can comprise a peptide fragment with at least about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% amino acid sequence homology to a corresponding peptide fragment of the parent transposase. The peptide fragment can be at least about 10, about 15, about 20, about 25, about 30, about 35, about 40, about 45, about 50, about 60, about 70, about 80, about 90, about 100, about 150, about 200, about 250, about 300, about 400, or about 500 amino acids in length. For example, a transposase derived from Tn5 can comprise a peptide fragment that is 50 amino acids in length and about 80% homologous to a corresponding fragment in a parent Tn5 transposase. In some cases, the insertion can be facilitated and/or triggered by addition of one or more cations. The cations can be divalent cations such as, for example, Ca²⁺, Mg²⁺ and Mn²⁺.

The adaptor molecules can comprise additional sequences that can be used for ligations, digestion, amplification, detection and/or sequencing. Such additional sequences can include, but are not limited to, sequencing adaptors, primer binding sites, locked nucleic acids (LNAs), zip nucleic acids (ZNAs), RNAs, affinity reactive molecules (e.g., biotin, dig), self-complementary molecules, phosphorothioate modifications, DNA tags, barcodes, and azide or alkyne groups. In some embodiments, the sequencing adaptors can further comprise a barcode label. Further, the barcode labels can comprise a unique sequence. The unique sequences can be used to identify the individual insertion events. Any of the tags can further comprise fluorescence tags (e.g., fluorescein, rhodamine, Cy3, Cy5, thiazole orange, etc.).

In some embodiments, the adaptor molecules can comprise unmodified DNA oligonucleotides. Examples of such unmodified DNA oligonucleotides include, but are not limited to, oligonucleotides consisting of the 19 basepair mosaic end Tn5 transposase recognition sequence, oligonucleotides which contain the recognition sequence as a subsequence as well as containing an additional sequence as a subsequence (e.g., Illumina Read 1 or Read 2 or any user-defined sequence). In some embodiments, the adaptor molecules can comprise modified DNA oligonucleotides. As used herein, “modified DNA oligonucleotides” refer to oligonucleotides which contain a chemical modification on the 5ʹ end, the 3ʹ end, or internally, and/or oligonucleotides that incorporate non-standard DNA bases (e.g., uracil, xeno-nucleic acids). Examples of such modified DNA oligonucleotides include, but are not limited to, 5ʹ or 3ʹ phosphorylation, 5ʹ acrydite modification, internal methacrylate functionalized uracil.

Additionally, the insertional enzyme complex can further comprise an affinity tag. In some cases, the affinity tag can be an antibody. The antibody can bind to, for example, a transcription factor, a modified nucleosome or a modified nucleic acid. Examples of modified nucleic acids include, but are not limited to, methylated or hydroxymethylated DNA. In other cases, the affinity tag can be a single-stranded nucleic acid (e.g., ssDNA, ssRNA). In some examples, the single-stranded nucleic acid can bind to a target nucleic acid. In further cases, the insertional enzyme complex can further comprise a nuclear localization signal.

In some embodiments, determining whether the tagged linearized fragment is derived from an eccDNA comprises amplifying the tagged linearized fragment of genomic DNA. Thus, in some embodiments, the method further comprises amplifying the tagged fragments of genomic DNA. The expression “amplification” or “amplifying” refers to a process by which extra or multiple copies of a particular polynucleotide are formed. The term “amplification product” refers to the nucleic acids, which are produced from the amplifying process as defined herein.

Amplification includes methods generally known to one skilled in the art such as, but not limited to, PCR, ligation amplification (or ligase chain reaction, LCR), real time (rtPCR) or quantitative PCR (qPCR), and other amplification methods. These methods are generally known. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In one embodiment, the ligation product is amplified using PCR. In general, the PCR procedure describes a method of gene amplification which comprises (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified. In some embodiments, the tagged fragments of genomic DNA are amplified using qPCR. Quantitative polymerase chain reaction is used to simultaneously detect a specific DNA sequence in a sample and determine the actual copy number of this sequence relative to a standard. In some embodiments, the tagged fragments of genomic DNA are amplified using rtPCR. In real-time PCR, the DNA copy number can be established after each cycle of amplification. By using a fluorescent reporter in the reaction, it is possible to measure DNA generation. Additional representative approaches for amplification are disclosed in the EXAMPLES set forth herein below.

In some embodiments, the determining whether the tagged linearized fragment is derived from an eccDNA comprises sequencing the tagged linearized fragment of genomic DNA. The fragments can be sequenced using any convenient method and can be sequenced prior to or after an amplification step, again using any convenient method. The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained. Sequencing can be carried out by any method known in the art including, but not limited to, sequencing by hybridization, sequencing by ligation or sequencing by synthesis. Sequencing by ligation includes, but is not limited to, fluorescent in situ sequencing (FISSEQ). Sequencing by synthesis includes, but is not limited to, reversible terminator chemistry (i.e. Illumina SBS). Other sequencing approaches are described in the EXAMPLES provided herein below.

In some embodiments, the tagged fragments can be sequenced to generate a plurality of sequencing reads. The fragments may be sequenced using a high-throughput sequencing technique. In some cases, the sequencing reads can be normalized based on the sequence insertion preference of the insertional enzyme. The sequencing reads can be used to determine the accessibility of the polynucleotide at any given site. In some embodiments, the length of the sequenced reads can be used to determine can be used to detect or determine a genomic region of origin for the eccDNA and/or can also be used to detect or determine the presence of a cell type, population of cells, and/or tissue type in the subject, such as the presence of cancer. For example, cancers are different from normal cells in having long eccDNA, e.g., 40% of eccDNA in cancers are at least about 1 kilobase (kb) and can range in length from about 1 kb to about a few Megabases (MB). Any method of high throughput sequencing can be used for sequencing the tagged eccDNA fragments e.g. Illumina paired-end reads, Nanopore sequencing from Oxford Nanopore or PacBio SMRT sequencing. These sequencing techniques can be used to obtain the length of the eccDNA and also the presence of particular nucleic acid sequences on the eccDNA, such as the presence of resistance genes on the eccDNA, as described in more detail elsewhere herein.

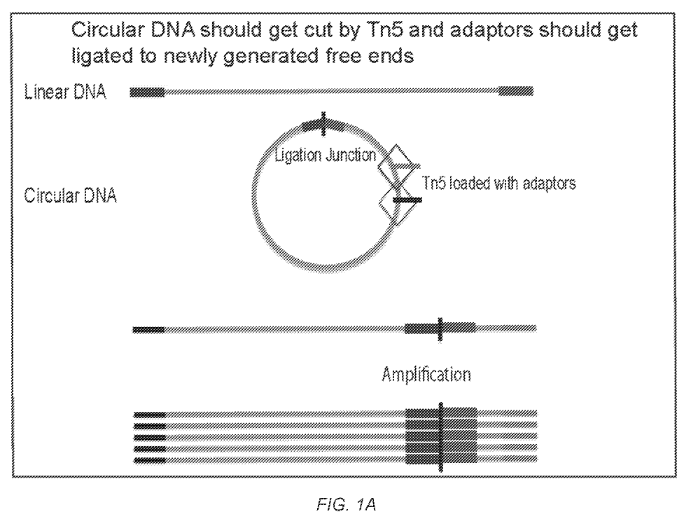

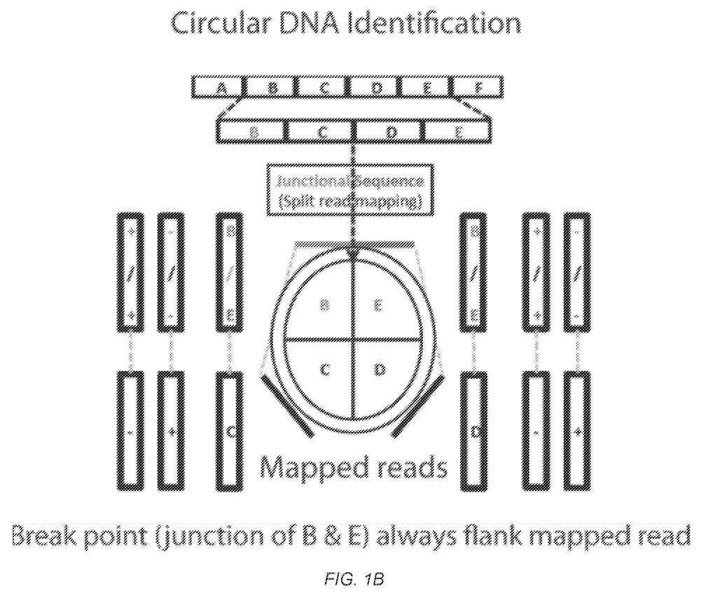

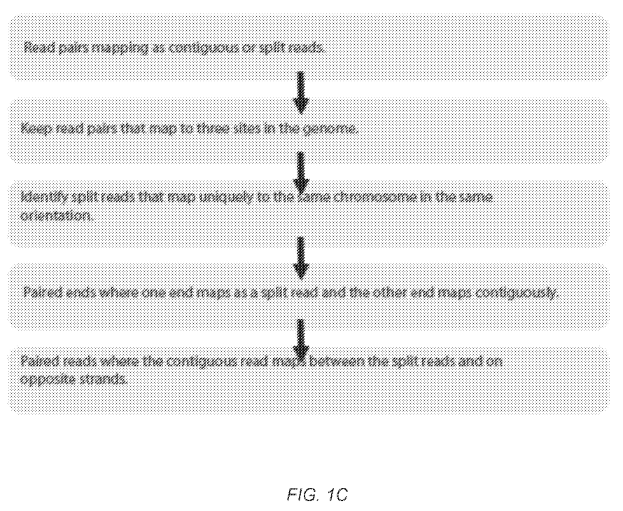

In some embodiments, determining whether the tagged linearized fragment is derived from an eccDNA comprises detecting a junctional sequence in the tagged linearized fragment. Detecting a junctional sequence can be carried out by any convenient method as would be apparent to one of ordinary skill in the art upon a review of the instant disclosure. In some embodiments, detecting a junctional sequence comprises employing read pairs. See, e.g. FIG. 1C. In some embodiments, mapped-unmapped pairs of reads are used. In mapped-unmapped pairs of reads, one read maps uniquely to the genome, but the other end is unmappable to the genome because it does not exist in the native genome. The approach, as might be employed using an algorithm (representative embodiments provided in the EXAMPLES, see, e.g., Table 11), then examines whether the unmappable read can be mapped by splitting read into two, where the two parts of the split read map to the genome on the “opposite strand” as to the mapped read, and flanking the mapped read. Since the paired-read sequencing is typically performed on DNA fragments of a defined length (usually 300-400 base), it is also ensured that at least one of the parts of the split read is <400 bases from the mapped read. In some embodiments, an alternate method for finding the circles, the discordant paired end read approach, is applied. The paired-end read (NGS) gives sequences of both ends of each DNA fragments and the distance between paired reads in sample genome should be approximately equal to library insert size. When both reads of the pair can be mapped to the reference genome, but they are mapped to different chromosomes, different orientations, or their coordinates may not agree with the library insert size, those read-pairs called discordant read-pairs. Specifically, when read-pairs are mapped to different orientation on same chromosome, they suggest presence of tandem duplication or circular DNA. Paired-reads with such a feature are extracted from whole reads. Additionally, paired reads can be employed to detect or determine a genomic region of origin for the eccDNA and/or can also be used to detect or determine the presence of a cell type, population of cells, and/or tissue type in the subject, such as the presence of cancer. The eccDNA are derived from transcriptionally active parts of the genome, which differ between tissue lineages. Thus, the chromosomal areas from which the eccDNA are derived may identify the tissue that the eccDNA arises from. eccDNA are also often enriched in CpG islands. The methylation status of CpG islands on the eccDNA can also be identified by available methods. Since the methylation of specific CpG islands also differ between tissue lineages, this is another method by which the presently disclosed subject matter can identify the lineage from which the eccDNA was derived.

In some embodiments, the biological sample can be permeabilized to allow access for an enzyme, such as an insertional enzyme. The permeabilization can be performed in a way to minimally perturb the nuclei in the sample. In some instances, the sample can be permeabilized using a permeabilization agent. Examples of permeabilization agents include, but are not limited to, NP40, digitonin, tween, streptolysin, and cationic lipids. In other instances, the sample can be permeabilized using hypotonic shock and/or ultrasonication. In other cases, the insertional enzyme can be highly charged, which may allow it to permeabilize through cell membranes.

A “sample”, as used herein, refers in some embodiments to a biological sample from a subject, including, but not limited to, normal tissue samples, diseased tissue samples, biopsies (solid and liquid), blood, saliva, feces, semen, tears, cerebrospinal fluid, sputum, bronchial washes and urine. A sample can also be any other source of material obtained from a subject which contains cells, tissues, or fluid of interest. A sample can also be obtained from cell or tissue culture. In some embodiments, the sample comprises a biopsy or a blood sample. However, any suitable sample as would be apparent to one of ordinary skill in the art upon a review of the instant disclosure can be analyzed. The terms “sample” and “biological sample” are used interchangeably herein and in a broad sense, and are intended to include sources that contain nucleic acids. Exemplary biological samples include, but are not limited to tissues, including but not limited to, liver, spleen, kidney, lung, intestine, thymus, colon, tonsil, testis, skin, brain, heart, muscle and pancreas tissue. Other exemplary biological samples include, but are not limited to, biopsies, bone marrow samples, organ samples, skin fragments and organisms. Materials obtained from clinical or forensic settings are also within the intended meaning of the term biological sample. Preferably, the sample is derived from a human, animal or plant. Preferably, the biological sample is a tissue sample, preferably an organ tissue sample. Preferably, samples are human. The sample can be obtained, for example, from autopsy, biopsy or from surgery. It can be a solid tissue such as, for example, parenchyme, connective or fatty tissue, heart or skeletal muscle, smooth muscle, skin, brain, nerve, kidney, liver, spleen, breast, carcinoma (e.g., bowel, nasopharynx, breast, lung, stomach etc.), cartilage, lymphoma, meningioma, placenta, prostate, thymus, tonsil, umbilical cord or uterus. The tissue can be a tumor (benign or malignant), cancerous or precancerous tissue. The sample can be obtained from an animal or human subject affected by disease or disorder or suspected of same (normal or diseased), or considered normal or healthy. In some embodiments, the tumor (benign or malignant), cancerous, or precancerous tissue is a tissue from any of the tissues set forth herein above, such as but not limited to, pancreatic cancer, breast cancer, prostate cancer, ovarian cancer, lung cancer, head and neck cancer, non-Hodgkin’s lymphoma, acute myelogenous leukemia, acute lymphoblastic leukemia, neuroblastoma, gliomas, and glioblastoma.

If desired, fixation of the biological sample can be effected with fixatives known to the person skilled in the art. In one embodiment, the fixative, includes but is not limited to, acids, alcohols, ketones or other organic substances, such as, glutaraldehyde, formaldehyde or paraformaldehyde. Examples of fixatives and uses thereof may be found in Sambrook et al. (1989). If employed, the used fixation also preserves DNA and RNA. Other fixatives and fixation methods for providing a fixed biological sample are known in the prior art. For example, the biological sample can be fresh froze, wherein alcohol based fixed samples can be used. In one embodiment, the fixed tissue may or may not be embedded in a non-reactive substance such as paraffin. Embedding materials include, but are not limited to, paraffin, mineral oil, non- water soluble waxes, celloidin, polyethylene glycols, polyvinyl alcohol, agar, gelatin, nitrocelluloses, methacrylate resins, epoxy resins or other plastic media. Thereby, one can produce tissue sections of the biological material suitable for histological examinations.

In some embodiments, the sample is treated with an exonuclease prior to treating the biological sample to produce a tagged linearized fragment of genomic DNA. Any suitable exonuclease as would be apparent to one of ordinary skill in the art may be used. Representative exonucleases are disclosed in the EXAMPLES. By way of example and not limitation, any DNA exonuclease with no endonuclease activity can be used. Suitable examples are commercially available through the website, https://www.biocompare.com/Search-Enzymes/?search=DNA+exonuclease. By way of specific example and not limitation, Exonuclease I and Exonuclease III from E. coli and/or Lambda Exonuclease can be employed. Thus, in some embodiments, an exonuclease is first used to remove any linear genomic DNA that may be contaminating the genome-derived eccDNA preparation. Then, the eccDNA is linearized such as by using an insertional enzyme complex, such as a transposon, or by a restriction enzyme and then ligated to sequencing primers. The linearized, tagged eccDNA is then sequenced.

In some embodiments, the subject is a subject suffering from a cancer or suspected to be suffering from a cancer or a subject having a genetic disease or disorder or suspected to have a genetic disease or disorder. In some embodiments, the subject having a genetic disease or disorder or suspected to have a genetic disease or disorder is a fetus and the sample comprises a maternal blood sample. In some embodiments, identifying a cell type in the subject, identifying a cell population, identifying a tissue type; and/or identifying a nucleic acid sequence on the eccDNA further comprises identifying a cancer or a genetic disease or disorder in the subject. For example, fetal eccDNA present in the maternal blood can be used to identify fetal genetic disorders, such as Down’s syndrome.

In some embodiments, the method further comprises choosing a therapy based on the identified cell type, cell population, tissue type, and/or nucleic acid sequence. For example, a therapeutic agent, dose level, or modality can be selected based on the identified cell type, cell population, tissue type, and/or nucleic acid sequence, and then administered. Non-limiting representative embodiments are disclosed in the EXAMPLES set forth herein below. Additional examples would be apparent on of ordinary skill in the art upon a review of the instant disclosure, and include but are not limited to for example, avoiding a drug for therapy of a particular patient’s cancer because a gene that confers resistance to the drug is already present in the cancer on an eccDNA and will be rapidly amplified to make the cancer resistant to said drug.

A representative therapy that can be chosen and administered is the administration of an effective amount of a pharmaceutical composition to treat a disease or disorder in the subject. Pharmaceutical compositions administered to a subject in need thereof by any number of routes including, but not limited to, intra-tumoral, topical, oral, intravenous, intramuscular, intra-arterial, intramedullary, intrathecal, intraventricular, transdermal, subcutaneous, intraperitoneal, intranasal, enteral, topical, sublingual, or rectal approaches.

In accordance with one embodiment, a method for treating a subject in need of such treatment is provided. The method comprises administering a pharmaceutical composition to a subject in need thereof. Pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active polypeptide, one or more acceptable carriers, and optionally other polypeptides or therapeutic agents.

For in vivo applications, the compositions of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

Where the administration of the composition is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multidose unit.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Controlled- or sustained-release formulations of a pharmaceutical composition of the presently disclosed subject matter may be made using conventional technology. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington’s Pharmaceutical Sciences, 18th ed., Mack Pub. Co., Easton, Pennsylvania, United States of America and/or Gennaro (ed.) (2003) Remington: The Science and Practice of Pharmacy, 20th edition Lippincott, Williams & Wilkins, Philadelphia, Pennsylvania, United States of America, each of which is incorporated herein by reference.

Typically, dosages of the compound of the presently disclosed subject matter which may be administered to an animal, in some embodiments a human, range in amount from 1 µg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multidose unit.

Compositions may be administered to, for example, a cell, a tissue, or a subject by any of several methods described herein and by others which are known to those of skill in the art.

The relative amounts of the active ingredient, the pharmaceutically acceptable carrier, and any additional ingredients in a pharmaceutical composition of the presently disclosed subject matter will vary, depending upon the identity, sex, age, size, and condition of the subject treated and further depending upon the route by which the composition is to be administered.

Other components such as preservatives, antioxidants, surfactants, absorption enhancers, viscosity enhancers or film forming polymers, bulking agents, diluents, coloring agents, flavoring agents, pH modifiers, sweeteners or taste-masking agents may also be incorporated into the composition. Suitable coloring agents include red, black, and yellow iron oxides and FD&C dyes such as FD&C Blue No. 2, FD&C Red No. 40, and the like. Suitable flavoring agents include mint, raspberry, licorice, orange, lemon, grapefruit, caramel, vanilla, cherry grape flavors, combinations thereof, and the like. Suitable pH modifiers include citric acid, tartaric acid, phosphoric acid, hydrochloric acid, maleic acid, sodium hydroxide, and the like. Suitable sweeteners include aspartame, acesulfame K, thaumatic, and the like. Suitable taste-masking agents include sodium bicarbonate, ionexchange resins, cyclodextrin inclusion compounds, adsorbates, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multidose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, and birds including commercially relevant birds such as chickens, ducks, geese, and turkeys.

In some embodiments, the presently disclosed subject matter provides a kit for detecting eccDNA. In some embodiments, the kit comprises one or more reagents suitable for carrying out a method in accordance with the presently disclosed subject matter, and instruction material for employing the one or more reagents.

As used herein, an “instructional material” includes a publication, a recording, a diagram, or any other medium of expression which can be used to communicate the usefulness of the methods of the presently disclosed subject matter in the kit for effecting the analyses recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains one or more reagents for carrying out the presently disclosed subject matter or be shipped together with a container which contains the one or reagents. Alternatively, the instructional material may be shipped separately from the container with the intention that the instructional material and the reagents be used cooperatively by the recipient.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al., 1989; Glover, 1985; Gait, 1984; Harlow & Lane, 1988; Roe et al., 1996; and Ausubel et al., 1995.

The presently disclosed subject matter may be embodied in other specific forms without departing from the spirit or essential attributes thereof. The presently disclosed subject matter encompasses all combinations of the different aspects of the presently disclosed subject matter noted herein. It is understood that any and all embodiments of the presently disclosed subject matter may be taken in conjunction with any other embodiment or embodiments to describe additional representative embodiments. It is also to be understood that each individual element of the disclosed embodiments is intended to be taken individually as its own independent representative embodiment. Furthermore, any element of an embodiment is meant to be combined with any and all other elements from any embodiment to describe an additional embodiment.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Example 1

### Principle of Circular DNA Identification by Tagmentation Method

EccDNAs are known to have chromosomal origin. A linear DNA fragment is generated either by the chromosome breakage due to adjoining DNA breaks, e.g. in chromothripsis (Maher et al., Cell 148, 29-32 (2012)), or by DNA synthesis related to DNA replication or repair. The two ends of a linear DNA are ligated to make a circular DNA (FIG. 1A), creating a specific junctional sequence that is not present in the normal reference genome. A very simple method is provided to identify eccDNAs by collecting all the read pairs where one read of a pair maps uniquely to the genome in a contiguous manner (<=5 bp insertions or deletions or substitutions) and the other read maps as a split read (non-contiguous segments that could be as far apart as a few MB, but usually are much closer) flanking the mapped read (FIG. 1B). The split read maps to the circular DNA ligation junction and the other (contiguously mapped read) maps to the body of the putative eccDNA. The start of the first split read and the end of second read are annotated as the start and end of the eccDNA. Tandem duplication of DNA in the genome will also create a similar junctional sequence, but for the purpose of identifying incipient gene amplification, an eccDNA or a tandem duplication of a chromosomal segment are equally important. However, if the aim is to exclusively and comprehensively identify eccDNA, the ATAC-seq library is prepared from eccDNA-enriched samples where linear DNA has been removed by exonuclease digestion, as also described elsewhere herein.

A representative technique or pipeline to identify eccDNA coming from one locus (non chimeric eccDNA) of any length is available through the following GitHub page (https://github.com/pk7zuva/Circle_finder (https://github.com/pk7zuva/Circle_finder/blob/master/circle_finder-pipeline-bwa-mem-samblaster.sh). The steps to find a circular DNA from any paired end high throughput-sequencing library are detailed in FIGS. 1B-1C and the Methods for the EXAMPLES provided herein below. A scrip for an algorithm is also provided herein below at Table 11

### Example 2

### Application of ATAC-Seq to Identify Circular DNA in OVCAR8 and C4-2B Cell Lines

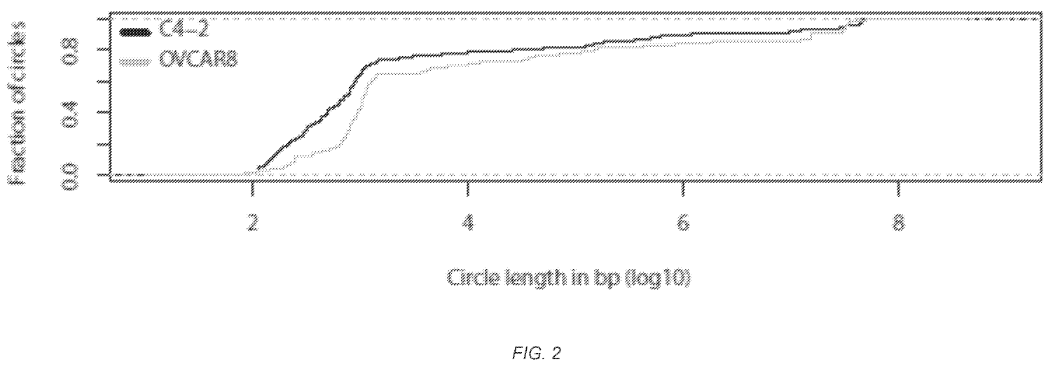

ATAC-seq libraries were prepared from C4-2B prostate cancer and OVCAR8 ovarian cancer cell lines. The sequencing and mapping statistics are given in Table 4; >90% of the reads mapped to human genome and the computational pipeline identified hundreds of circular DNA. The length distribution of eccDNA is shown in FIG. 2: around 68% in C4-2B and 37% in OVCAR8 of eccDNA are <1 kb, and so are similar to the microDNAs that were identified earlier in normal and cancer cells (4, 5). However, 32% of the eccDNA in C4-2 and 63% in OVCAR8 are >1 kb, including eccDNAs long enough to encode gene segments or even complete genes. The eccDNA are derived from all the chromosomes (Tables 7A and 7B). As a positive control hundreds of junctional sequences from the circular mitochondrial genome contaminating the nuclear preparations were identified.

### Example 3

### Validation of eccDNA Identified in C4-2 and OVCAR8 Cells by Inverse PCR

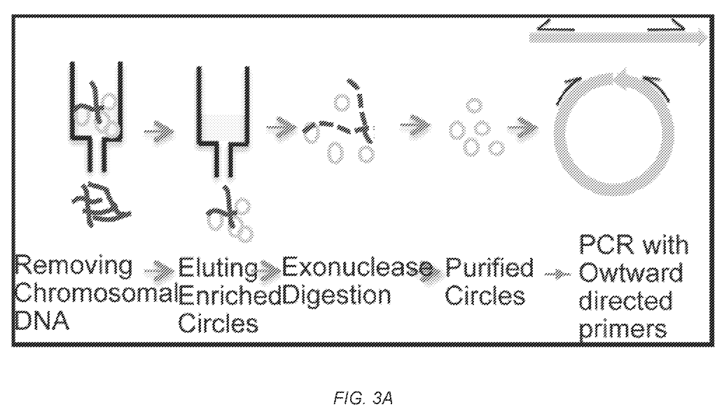

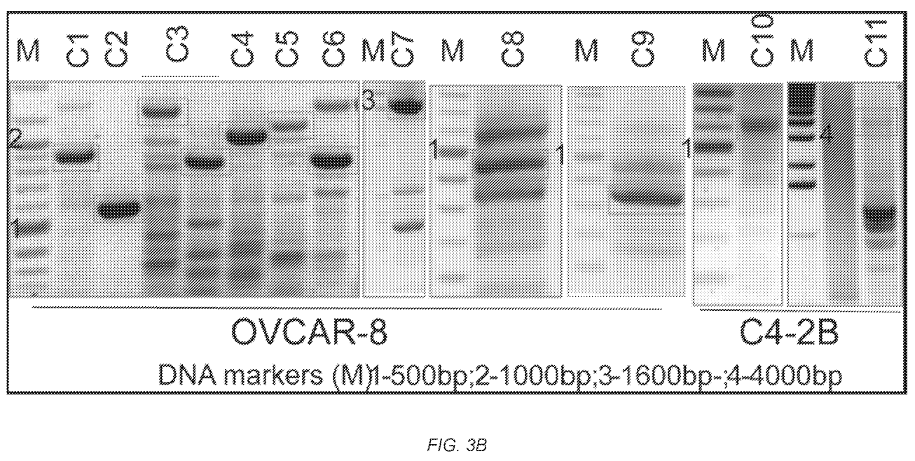

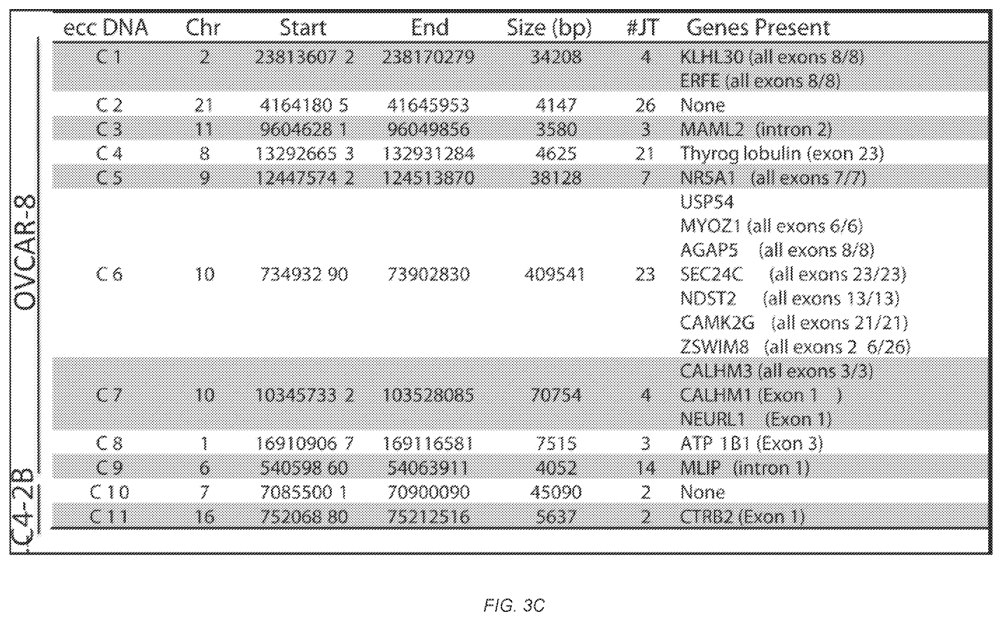

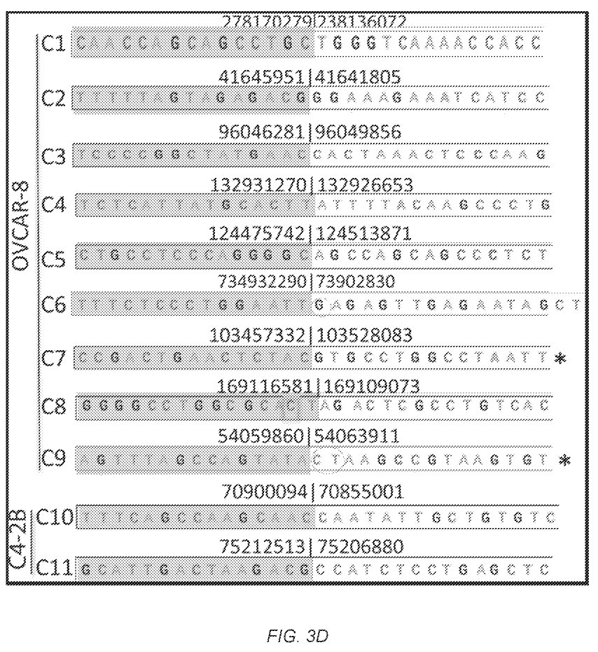

To confirm that the identified junctions are genuinely from eccDNA, and not from tandem genome duplications, circular DNA were isolated by a previously described method that relies on column chromatography and exonuclease digestion to remove all linear DNA and enrich eccDNA (FIG. 3A (5); see Methods for more details). Inverse PCR was performed with primers designed to amplify across the junctions of eccDNAs from C4-2B & OVCAR8 cell lines (FIG. 3B). 11 eccDNAs from OVCAR8 and 6 from C4-2B were tested. 9 of the 11 targets from OVCAR8 and 2 of the 6 from C4-2B gave amplicons of expected sizes (FIGS. 3B-3C). Sanger sequencing of the amplicons confirmed the junctional sequences identified by ATAC-seq (FIG. 3D and Table 2 immediately below). A fraction of the primers (2 in OVCAR8 and 4 in C4-2B) did not give desired amplicons possibly because of their presence in low complexity regions or because they came from tandem linear chromosomal duplications which did not survive column chromatography and exonuclease digestion. The junctional sequences in FIG. 3D and repeated below are unique to the eccDNA. The two halves of the junctional sequence are present in the genome, but they are separate from each other. Numbers indicate chromosomal location on respective chromosomes C1-C11, full sequences provided in the Sequence Listing as follows: C1, SEQ ID NO: 12; C2, SEQ ID NO: 13; C3, SEQ ID NO: 14; C4, SEQ ID NO: 15; C5, SEQ ID NO: 16; C6, SEQ ID NO: 17; C7, SEQ ID NO: 18; C8, SEQ ID NO: 19; C9, SEQ ID NO: 20; C10, SEQ ID NO: 21; C11, SEQ ID NO: 22.

Table 2

Junctional sequences - FIG. 3D
2781702791238136072
C1: CAACCAGCAGCCTGC | TGGGTCAAAACCACC (SEQ ID NO: 1)
41645951141641805
C2: TTTTTAGTAGAGACG | GGAAAGAAATCATCC (SEQ ID NO: 2)
96046281196049856
C3: TCCCCGGCTATGAAC | CACTAAACTCCCAAG* (SEQ ID NO: 3)
1329312701132926653
C4: TCTCATTATGCACTT | ATTTTACAAGCCCTG (SEQ ID NO: 4)
124475742 | 124513871
C5: CTGCCTCCCAGGGGC | AGCCAGCAGCCCTCT* (SEQ ID NO: 5)
734932290 | 73902830
C6: TTTCTCCCTGGAATT | GAGAGTTGAGAATAGCT* (SEQ ID NO: 6)
103457332 | 103528083
C7: CCGACTGAACTCTAC | GTGCCTGGCCTAATT* (SEQ ID NO: 7)
169116581 | 169109073
C8:GGGGCCTGGCGCACT | AGACTCGCCTGTCAC (SEQ ID NO: 8)
54059860154063911
C9: AGTTTAGCCAGTATA | CTAAGCCGTAAGTGT* (SEQ ID NO: 9)
70900094 | 70855001
C10: TTTCAGCCAAGCAACICAATATTGCTGTGTC (SEQID NO: 10)
75212513175206880
C11: GCATTGACTAAGACG | CCATCTCCTGAGCTC (SEQ ID NO: 11)

### Example 4

### Validation of eccDNA by Metaphase Fish in OVCAR8 Cells

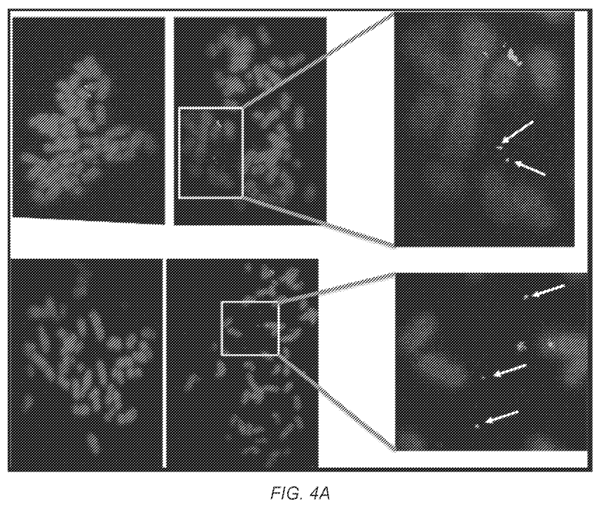

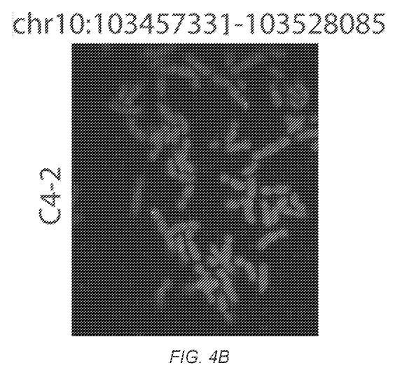

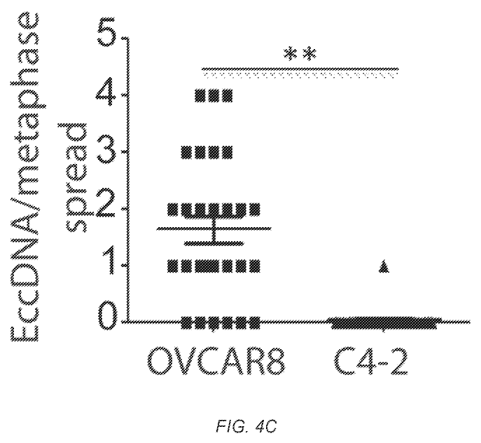

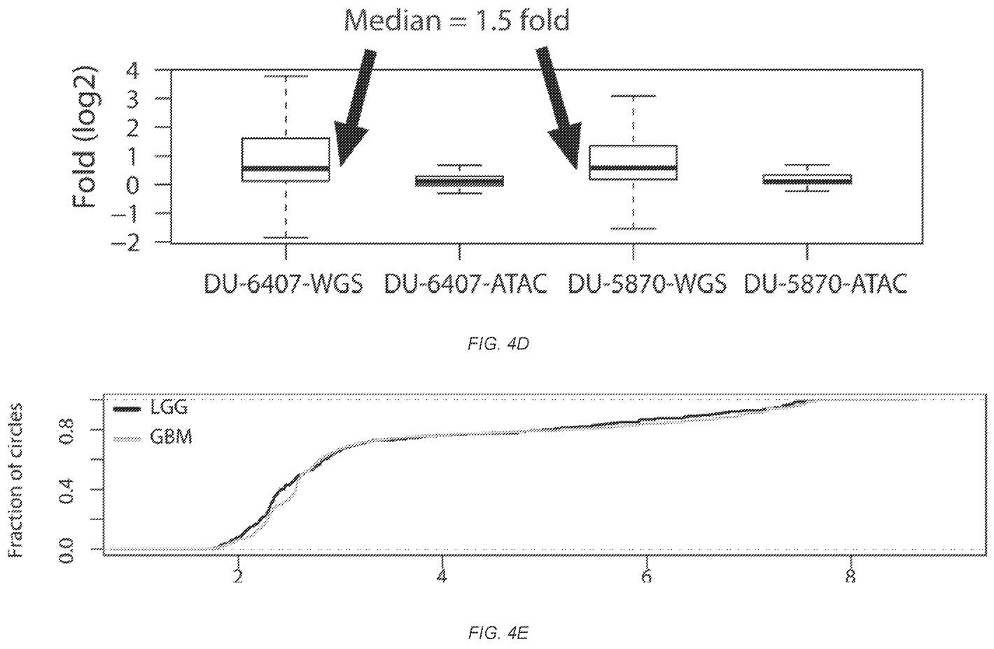

An independent method for ascertaining whether a locus identified in this study is in an extrachromsomal DNA is to carry out FISH on metaphase spreads. This analysis was performed with two loci that were predicted to be present as either an eccDNA or a gene duplication in OVCAR8 cells but not in C4-2 cells: chr2: 238136071-238170279 and chr10: 103457331-103528085. Both were confirmed by inverse PCR in FIG. 3 (C1 and C7). Signal was detected off the main chromosomes in some of the metaphase spreads, but not others (FIG. 4A), consistent with the hypothesis that the junctional sequences identify somatically mosaic eccDNA in this cell line. For negative control C4-2 (FIG. 4B), the spreads do not show an extrachromosomal DNA signal. The 71 kb eccDNA in OVCAR8 (n = 28) and C4-2 (n = 24) (negative control) metaphase spreads were quantified for locus chr10: 103457331-103528085 and shown in the graph (FIG. 4C).

### Example 5

### Identification of eccDNA from ATAC-seq Data for Glioblastoma (GBM) Cell Lines

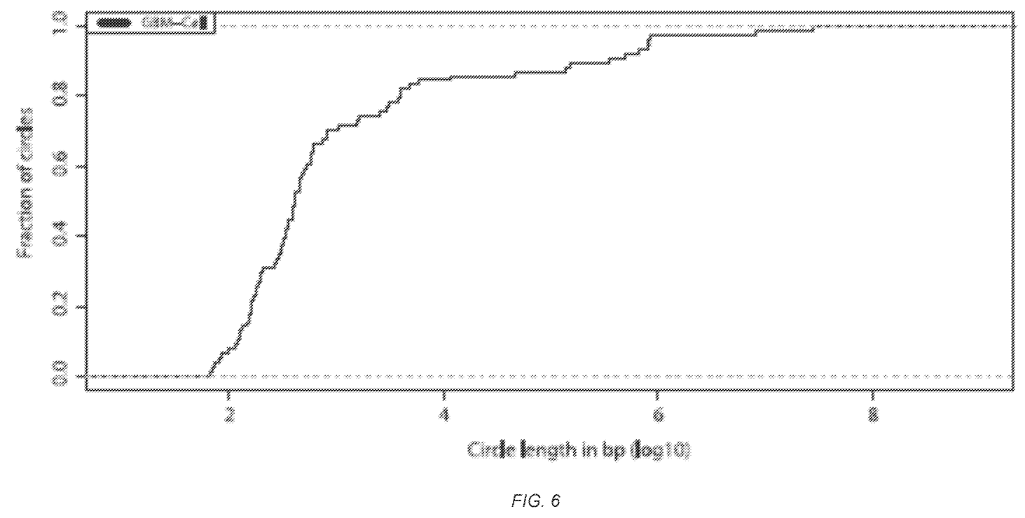

Epidermal growth factor receptor (EGFR) was one of the first oncogenes identified in brain cancer and is massively amplified in some GBM patients (Libermann et al., Nature 313, 144-147 (1985)). This somatic copy number variation is present in 43% of GBM patients (Maire et al., Neuro Oncol 16 Suppl 8, viii1-6 (2014)) . Recent studies have provided further evidence that this oncogenic amplification occurs on eccDNA (deCarvalho et al., Nat Genet 50, 708-717 (2018); Turner et al., Nature 543, 122-125 (2017); Xu et al., Acta Neuropathol 137, 123-137 (2019)) . To check if the eccDNA can be detected in ATAC-seq data generated from GBM cell lines, six ATAC-seq libraries generated from GBM cell lines developed from a single glioblastoma patient (Xie et al., Cell 175, 1228-1243 e1220 (2018)) were assessed. The Circle_finder pipeline was run, combining all the six libraries (GSM3318539, GSM3318540, GSM3318541, GSM3318542, GSM3318543 and GSM3318544) and 58 eccDNAs were found, varying in size from few hundred bases to few megabases. The length distribution and chromosomal distribution of identified eccDNAs are shown in FIG. 6 and Table 9. Of note, eccDNA harboring the EGFR gene was the most abundant eccDNA. The top five most-abundant eccDNAs (or tandem gene duplications) identified were chr4:118591708-119454712 (METTL14, SEC24D, SYNPO2, MYOZ2, USP53, C4orf3, FABP2), chr7:54590796-55256528 (SEC61G, EGFR), chr7:54771165-54782815(No protein coding genes), chr7:65038261-65873269 (transcribed unprocessed pseudogenes), chr7:65038264-65873256 (transcribed unprocessed pseudogenes).

### Example 6

### Application to GBM and LGG TCGA ATAC-Seq Data

Having demonstrated above that ATAC-seq data can be repurposed to identify eccDNA, attention was turned to ATAC-seq data generated by TCGA consortium (Corces et al., Science 362, Issue 6413, eaav1898 (2018)) with a primary focus on two LGGs for which whole genome sequencing data and ATAC-seq data was available. In the TCGA-DU-5870-02A ATAC-seq library 21 eccDNAs (junctional tag >=2; 13>1 kb, 7>50 kb) were found. In the TCGA-DU-5870-02A WGS library 637 eccDNAs (junctional tag >=2; 361>1 kb, 105>50 kb) were found. The eccDNAs identified in ATAC-seq and WGS libraries were further compared and 21 common eccDNAs (junctional tag>=1; Table 5) were found.

In the ATAC-seq library from TCGA-DU-6407-02B 64 eccDNAs (junctional tag >=2; 21>1 kb, 15>50 kb) were found and in WGS libraries from the same tumor 455 eccDNAs (junctional tag >=2; 307>1 kb, 131>50 kb) were found. 44 common eccDNAs were identified in both libraries (junctional tag>=1; Table 6). Many of common eccDNAs had a high number of junctional tags in the WGS library, perhaps a surrogate marker of their abundance.

A higher number of eccDNA/duplication events was seen in WGS compared to ATAC-seq, but 21 and 44 eccDNAs were common between ATAC-seq and WGS in TCGA-DU-5870-02A and TCGA-DU-6407-02B libraries respectively (Tables 5-6). The lack of more overlap between the eccDNAs identified by ATAC-seq and WGS from even the same tumor is most likely due to somatic mosaicism (a) because different sections are used for the two libraries and (b) because of insufficient depth of sequencing in either library.

As mentioned earlier, the Circle_finder algorithm cannot distinguish between an extrachromosomal circle and chromosomal segmental tandem duplication without experimentally purifying the circles before library preparation, and so these loci are referred to as eccDNA/duplication. The signal for the eccDNA/duplication detected from WGS data was strong in the two tumors and was also evident in a targeted Copy Number Analysis from the WGS data (not a genome-wide analysis). The median sequencing read coverage at the eccDNA/duplication loci was 1.5 fold higher compared to equivalent upstream or downstream regions, suggesting that at least a two-fold amplification of one allele occurred in at least 50% of the cells. Surprisingly, the eccDNA/duplication events detected by ATAC-seq did NOT show corresponding amplification in WGS (FIG. 4D). This result suggests that as with eccDNAs detected by rolling circle amplification, the eccDNA/duplication events identified by ATAC-seq are somatically mosaic in the GBM cell lines and are detected even before a CNV is apparent from whole genome sequencing of a large population of tumor cells.

10 LGG and 8 GBM ATAC-seq libraries were next analyzed and a total of 2152 and 3147 eccDNA/duplication events were found in LGG and GBM samples, respectively. The length distribution of eccDNA/duplications is shown in FIG. 4E. 58% of the loci are <1 kb (similar to microDNA), but nearly 41% (2200 eccDNAs in GBM+LGG) are 50 kb to 50 MB in length, suggesting that they harbor full length genes. The chromosomal distribution of eccDNA identified (Junctional Tags>=2) in LGG and GBM samples is shown in Tables 8A and 8B. The EGFR locus was contained in the eccDNA/duplication identified in GBM patients, supporting that the use of Circle_finder in ATAC-seq data can identify loci that have been amplified even in a subset of the cells in the tumor.

### Example 7

### Cumulative Analysis of All Small eccDNA (microDNA)

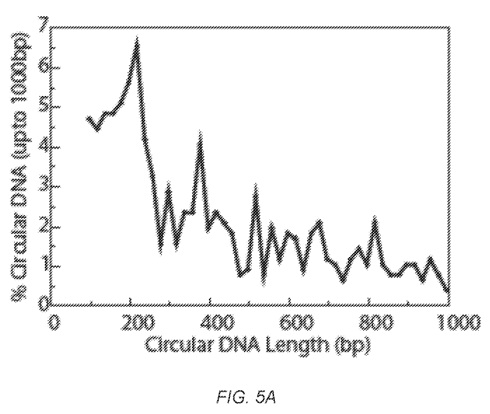

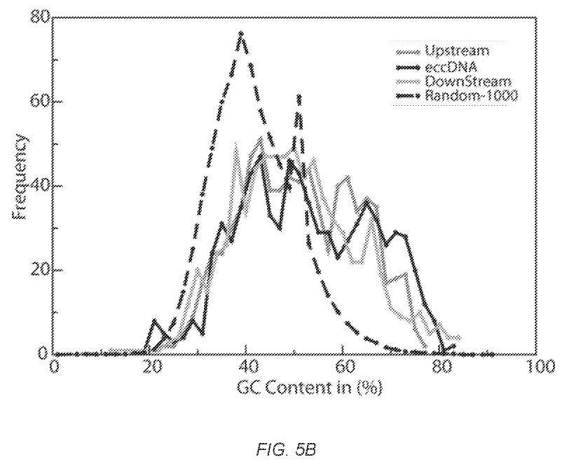

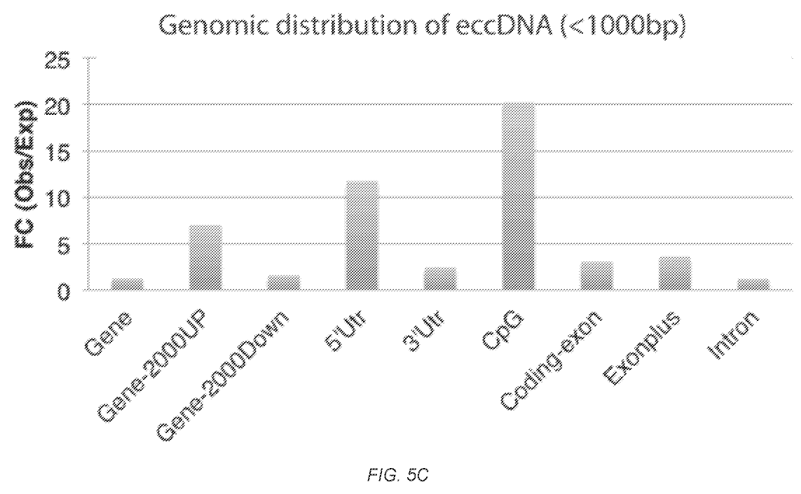

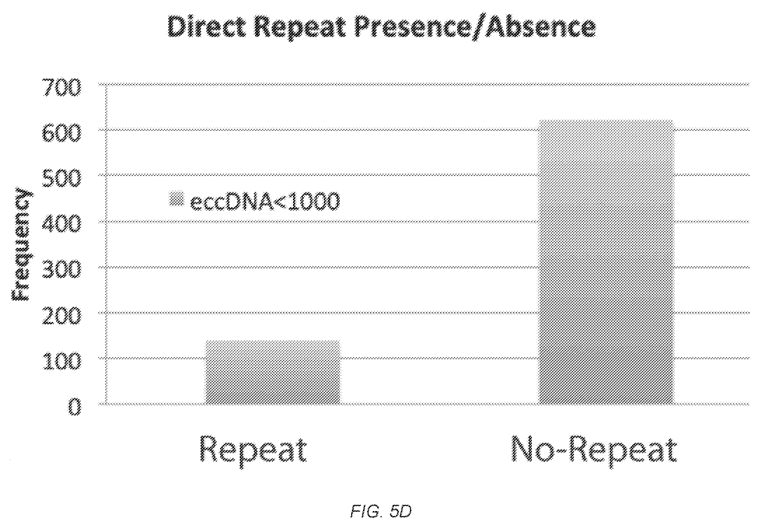

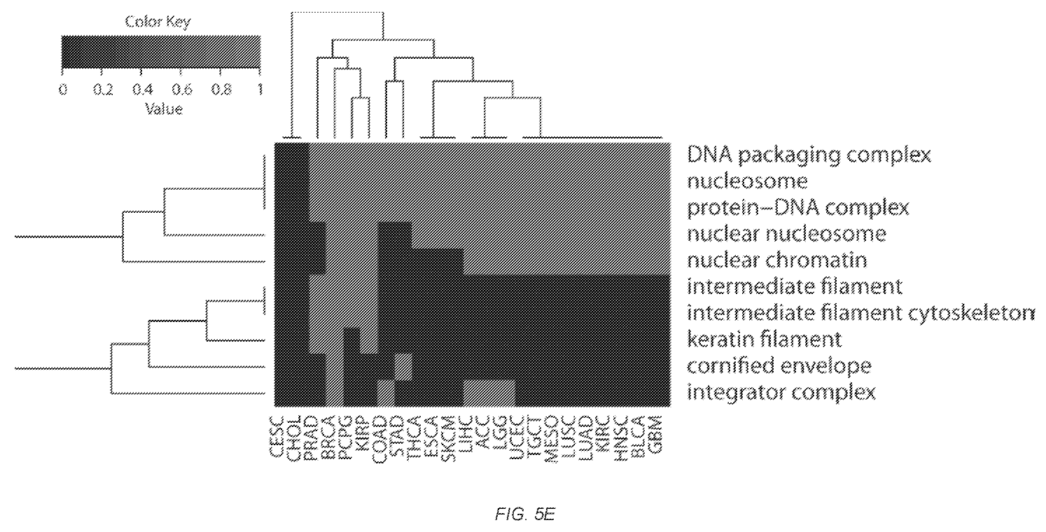

After pooling all the eccDNA identified so far in Examples 1-6 (OVCAR8 + C4-2 + 8 GBM + 10 LGG), the ones <1 kb were analyzed to compare their properties with the microDNA identified earlier by rolling circle amplification (Kumar et al., Mol Cancer Res 15, 1197-1205 (2017); Shibata et al., Science 336, 82-86 (2012)). 4073 eccDNA were found that were <1 kb. The length distribution of these circles reveals characteristic peaks at about 200 and about 400 bases (FIG. 5A), that have been noted earlier. The higher GC content relative to the genome average (FIG. 5B), and the enrichment of the microDNA from upstream of genes, 5ʹUTR and CpG islands (FIG. 5C) is also similar to previous reports. Finally, around 15% of the small eccDNAs reported here appear to have used flanking sequences of 2-15 base micro homology (FIG. 5D) to promote the ligation that gives rise to the circle.

### Example 8

### Pan Cancer Analysis of eccDNA in TCGA ATAC-Seq Data

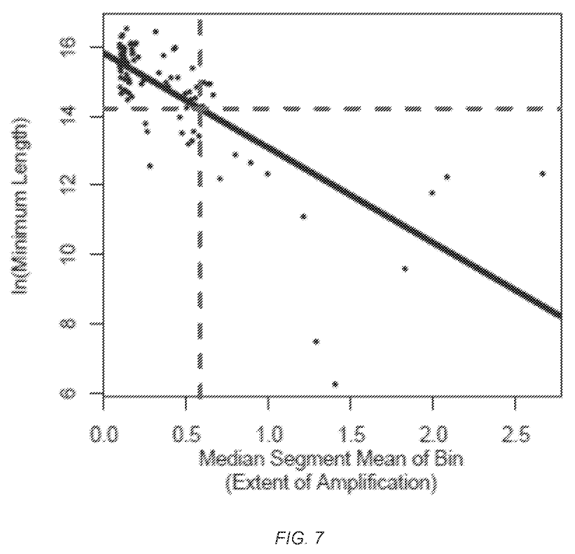

Finally, 360 ATAC-seq libraries from twenty-three tumor types generated by TCGA consortium were analyzed (FIG. 6 and Table 9, and FIG. 7; Tables 4-6) (Corces et al., Science 362, Issue 6413, eaav1898 (2018)). A total of 18,143 eccDNAs/duplications were found, of which 86% were <1 kb. The co-ordinates of eccDNA identified in each library are available through the webpage (http://genome.bioch.virginia.edu/TCGA-ATACSEQ-ECCDNA. The unique eccDNA intervals were used to extract the full length genes harbored inside the circle. The cancer driver genes (Bailey et al., Cell 174, 1034-1035 (2018)) amplified as eccDNA/duplication in individual tumor type is given in Table 3. Gene ontology analysis of all the genes carried on the eccDNA/duplication loci shows that Pathways related to nucleosomal events are significantly enriched in these loci (FIG. 5E).

TABLE 3

Known Cancer Driver Genes Amplified in eccDNA/Gene Duplications (JTGE1) in Various Tumor Types

| | |
|---|---|
| ACC | FGFR2, H3F3A, FOXA1, SMARCA4, NFE2L2, PMS1, SF3B1, SOS1, PCBP1, KIT, EGFR, GNAQ |
| BLCA | ERCC2, GRIN2D, PPP2R1A, SOS1, PCBP1, MSH3 |
| BRCA | FGFR2, MTOR, WT1, SF1, CCND1, PTPRC, PTPN11, KRAS, H3F3A, ERBB3, KLF5, MACF1, AKT1, FOXA1, MAP2K1, IDH2, ERBB2, SPOP, SETBP1, SMARCA4, CACNA1A, PIK3R2, GNA11, ERCC2, GRIN2D, PPP2R1A, U2AF1, NFE2L2, PMS1, SF3B1, IDH1, MAPK1, SOS1, PCBP1, CTNNB1, RHOA, FBXW7, KIT, MSH3, PIK3CG, UNCX, BRAF, CUL1, EGFR, GTF2I, MYC, SOX17, GNAQ, EIF1AX |
| CESE | MTOR, PTPRC, H3F3A, NFE2L2, PMS1, GNAQ |
| COAD | FGFR2, MTOR, WT1, SF1, CCND1, PTPRC, KRAS, H3F3A, ERBB3, CDK4, CHD4, ARID1A, KLF5, MACF1, FOXA1, MAP2K1, IDH2, ERBB2, SPOP, SETBP1, SMARCA4, CACNA1A, PIK3R2, GNA11, ERCC2, GRIN2D, PPP2R1A, GNAS, U2AF1, NFE2L2, PMS1, SF3B1, IDH1, MAPK1, PLXNB2, SOS1, PCBP1, PIK3CA, CTNNB1, RHOA, MSH3, EEF1A1, PIK3CG, BRAF, CUL1, EGFR, GTF2I, SOX17, GNAQ, EIF 1AX |
| ESCA | FGFR2, CCND1, PTPN11, KRAS, ERBB3, CDK4, KLF5, FOXA1, MAP2K1, IDH2, ERBB2, SETBP1, NFE2L2, PMS1, SF3B1, IDH1, SOS1, PCBP1, PIK3CA, CTNNB1, RHOA, KIT, MSH3, EEF1A1, EGFR, GTF2I, MYC, SOX17 |
| GBM | H3F3A, CDK4, PIK3R2, ERCC2, GRIN2D, EGFR, MYC |
| HNSC | FGFR2, MTOR, SF1, CCND1, PTPRC, KRAS, ERBB3, CDK4, KLF5, MAP2K1, ERBB2, SPOP, SETBP1, KEAP1, SMARCA4, CACNA1A, PIK3R2, ERCC2, GRIN2D, NFE2L2, PMS1, SF3B1, IDH1, PCBP1, PIK3CA, EGFR, GTF2I, MYC, GNAQ |
| KIRC | FGFR2, MTOR, WT1, PTPRC, KRAS, ERBB3, CDK4, FOXA1, MAP2K1, ERBB2, SPOP, SMARCA4, CACNA1A, PIK3R2, ERCC2, GRIN2D, PPP2R1A, NFE2L2, PMS1, SF3B1, IDH1, MAPK1, SOS1, PCBP1, PIK3CG, RAC1, SOX17 |
| KIRP | FGFR2, PTPRC, FOXA1, IDH2, ERBB2, SPOP, SMARCA4, ERCC2, GRIN2D, PPP2R1A, MAPK1, SMARCB1, PCBP1, PIK3CA, MSH3, PIK3CG, MET, BRAF, CUL1, EGFR, MYC, SOX17, GNAQ |
| LGG | WT1, MACF1, PCBP1, KIT, PIK3CG, MTOR |
| LIHC | WT1, SF1, CCND1, DHX9, PTPRC, PTPN11, KRAS, CHD4, MACF1, MAP2K1, ERBB2, SPOP, SETBP1, SMARCA4, CACNA1A, PIK3R2, GNA11, ERCC2, GRIN2D, U2AF1, PMS1, SF3B1, IDH1, SOS1, XPO1, PCBP1, PIK3CA, KIT, CDKN1A, EEF1A1, PIK3CG, BRAF, CUL1, GNAQ, EIF1AX |
| LUAD | FGFR2, MTOR, WT1, SF1, CCND1, PTPRC, KRAS, H3F3A, ERBB3, CDK4, KLF5, MACF1, MAP2K1, ERBB2, SPOP, SETBP1, SMARCA4, CACNA1A, PIK3R2, GNA11, ERCC2, GRIN2D, PPP2R1A, NFE2L2, PMS1, SF3B1, IDH1, SOS1, PCBP1, CTNNB1, RHOA, MSH3, EEF1A1, RAC1, GTF2I, MYC |
| LUSC | NRAS, PTPN11, KRAS, H3F3A, ERBB3, CDK4, ERCC2, GRIN2D, PPP2R1A, U2AF1, SF3B1, IDH1, SOS1, PCBP1, FGFR3, MSH3, GNAQ |
| MESO | FGFR2, PTPRC, PTPN11, ERCC2, GRIN2D, PPP2R1A, IDH1, CTNNB1, RHOA, PIK3CG, RAC1, GTF2I, MYC |
| PCPG | MTOR, NRAS, PTPRC, PMS1, SF3B1, IDH1, SOS1, EPAS1, PIK3CG, EGFR, MYC, SOX17 |
| PRAD | MTOR, KRAS, ERBB3, CDK4, CHD4, KLF5, FOXA1, ERBB2, SPOP, TP53, SETBP1, SMARCA4, CACNA1A, PIK3R2, GNA11, ERCC2, GRIN2D, PPP2R1A, NFE2L2, PMS1, SF3B1, IDH1, SOS1, PCBP1, KIT, MSH3, PIK3CG, EGFR, GTF2I, MYC, SOX17 |
| SKCM | CCND1, KRAS, CHD4, FOXA1, SETBP1, SMARCA4, CACNA1A, PIK3R2, ERCC2, GRIN2D, PPP2R1A, MAPK1, CTNNB1, RHOA, EGFR, EIF1AX |
| STAD | MTOR, NRAS, WT1, SF1, CCND1, KRAS, H3F3A, ERBB3, CDK4, MAP2K1, ERBB2, SPOP, SETBP1, SMAD4, SMARCA4, CACNA1A, PIK3R2, GRIN2D, NFE2L2, PMS1, SF3B1, IDH1, SOS1, PCBP1, PIK3CA, CTNNB1, RHOA, MSH3, CUL1, EGFR, GTF2I, MYC, SOX17, CNBD1, GNAQ, FAM46D |
| TGCT | WT1, KRAS, CHD4, MACF1, MAP2K1, IDH2, SETBP 1, ERCC2, GRIN2D, PPP2R1A, SOS1, CTNNB1, SOX17, EIF1AX |
| THCA | NRAS, KLF5, ERBB2, SPOP, ERCC2, GRIN2D, PPP2R1A, PCBP1, CTNNB1, RHOA, PIK3CG, GTF2I |
| UCEC | CCND1, KLF5, ERBB2, SPOP, PIK3R2, U2AF1, PCBP1, PIK3CA, PIK3CG |

TABLE 4

Summary of eccDNA Sequencing and Mapping to the Human Genome in C4-2 and OVCAR8 Cell Lines

| | C4-2 | OVCAR8 |
|---|---|---|
| Total PE150 reads | 374,931,197 | 346,833,854 |
| Mapped in pairs | 372,561,742 | 345,127,010 |
| Mapped-Reads | 373,585,003 | 343,541,464 |
| Un-Mapped-Reads | 1346194 | 1,706,844 |
| #eccDNA | 1,335 | 611 |

TABLE 5

Common eccDNA between WGS & ATACseq Libraries in TCGA-DU-5870-02A

| | JT-ATAC | JT-WGS | Length |
|---|---|---|---|
| chr12:8208854-8238302 | 1 | 13 | 29448 |
| chr13:42373132-42373518 | 1 | 12 | 386 |
| chr14:56200142-56200312 | 2 | 4 | 170 |
| chr14:99811139-99811333 | 2 | 1 | 194 |
| chr15:52523965-52524853 | 1 | 16 | 888 |
| chr1:60822423-60822666 | 1 | 7 | 243 |
| chr16:69820782-69825118 | 3 | 15 | 4336 |
| chr17:80024303-80024653 | 1 | 11 | 350 |
| chr18:79200703-79200779 | 1 | 2 | 76 |
| chr19:17722272-17722449 | 1 | 3 | 177 |
| chr2:110101070-110104430 | 1 | 14 | 3360 |
| chr22:49226585-49228448 | 2 | 11 | 1863 |
| chr3:42898790-46004380 | 1 | 1 | 3105590 |
| chr4:150242941-150244988 | 7 | 15 | 2047 |
| chr4:156660547-156660686 | 1 | 1 | 139 |
| chr6:167516501-167516632 | 1 | 1 | 131 |
| chr6:54059859-54063911 | 2 | 19 | 4052 |
| chr7:24028695-24028902 | 1 | 3 | 207 |
| chr7:47669120-47669934 | 1 | 10 | 814 |
| chr7:65038315-65873352 | 3 | 3 | 835037 |
| chr9:14692642-14692781 | 1 | 1 | 139 |

TABLE 6

Common eccDNA between WGS & ATACseq Libraries in TCGA-DU-5870-02A

| | JT-ATAC | JT-WGS | Length |
|---|---|---|---|
| chr10:95447028-95448268 | 1 | 7 | 1240 |
| chr11:28824878-36215494 | 1 | 16 | 7390616 |
| chr11:29133708-29430833 | 1 | 4 | 297125 |
| chr11:31877072-36188811 | 3 | 13 | 4311739 |
| chr11:36202059-36238115 | 2 | 28 | 36056 |
| chr11:36214300-36237662 | 1 | 9 | 23362 |
| chr11:36412110-36417948 | 2 | 13 | 5838 |
| chr11:36412110-36417951 | 2 | 13 | 5841 |
| chr1:14109813-14112070 | 1 | 1 | 2257 |
| chr13:113232110-113232386 | 1 | 1 | 276 |
| chr13:35957905-35958285 | 1 | 3 | 380 |
| chr13:42373132-42373518 | 1 | 15 | 386 |
| chr13:76789088-76789267 | 1 | 1 | 179 |
| chr14:100526783-100526942 | 1 | 1 | 159 |
| chr1:56530360-56530674 | 1 | 2 | 314 |
| chr15:91440230-91446387 | 1 | 13 | 6157 |
| chr17:41632757-41633279 | 1 | 1 | 522 |
| chr17:80024303-80024653 | 1 | 1 | 350 |
| chr20:2236337-2236458 | 2 | 19 | 121 |
| chr2:11554-91086 | 1 | 10 | 79532 |
| chr2:16052063-16452027 | 1 | 9 | 399964 |
| chr2:16177696-17516120 | 6 | 157 | 1338424 |
| chr2:16225124-16226720 | 8 | 195 | 1596 |
| chr2:16567034-17412029 | 1 | 1 | 844995 |
| chr2:16632426-17084537 | 1 | 13 | 452111 |
| chr2:16741536-17706105 | 2 | 56 | 964569 |
| chr2:17279980-17334701 | 7 | 15 | 54721 |
| chr2:17296668-17297551 | 3 | 14 | 883 |
| chr2:17584356-17877776 | 2 | 18 | 293420 |
| chr22:27805472-27805771 | 3 | 2 | 299 |
| chr2:63459820-63461139 | 1 | 6 | 1319 |
| chr3:5565190-5565271 | 1 | 1 | 81 |
| chr3 :95749272-95752156 | 1 | 9 | 2884 |
| chr4:52081945-52082092 | 1 | 1 | 147 |
| chr5: 168031041-168034576 | 3 | 5 | 3535 |
| chr6:118690555-118692765 | 1 | 6 | 2210 |
| chr6:118940825-118941155 | 1 | 19 | 330 |
| chr6:54059859-54063911 | 1 | 9 | 4052 |
| chr7:116674913-121917354 | 10 | 44 | 5242441 |
| chr7:482867-483641 | 1 | 11 | 774 |
| chr7:76498793-76998017 | 2 | 1 | 499224 |
| chr8: 145036620-145051835 | 1 | 15 | 15215 |
| chrX: 105007860-105008004 | 1 | 1 | 144 |
| chrY: 10945178-11295108 | 2 | 11 | 349930 |

TABLE 7A

C4-2-Circle-Co-ordinate

| Chromosome | Start | End |
|---|---|---|
| chr1 | 1067955 | 1068745 |
| chr1 | 4144482 | 4144633 |
| chr1 | 4939419 | 4939672 |
| chr1 | 4939548 | 4939686 |
| chr1 | 5160160 | 5160331 |
| chr1 | 5387170 | 5387379 |
| chr1 | 5485839 | 5486113 |
| chr1 | 8026890 | 8027022 |
| chr1 | 9411125 | 9411306 |
| chr1 | 10713835 | 14744297 |
| chr1 | 11039547 | 11041749 |
| chr1 | 16049926 | 16059816 |
| chr1 | 33180948 | 33181691 |
| chr1 | 55002896 | 55003057 |
| chr1 | 61082818 | 61083762 |
| chr1 | 67685357 | 67686391 |
| chr1 | 67685367 | 67686401 |
| chr1 | 67685375 | 67686409 |
| chr1 | 155324504 | 155325367 |
| chr1 | 226024126 | 235409833 |
| chr1 | 230751770 | 230752653 |
| chr1 | 230751780 | 230752663 |
| chr1 | 230751795 | 230752666 |
| chr1 | 235078245 | 235078548 |
| chr10 | 41887628 | 41896968 |
| chr10 | 75352105 | 75352337 |
| chr10 | 84385236 | 94699052 |
| chr10 | 93635610 | 94820838 |
| chr10 | 95447028 | 95448267 |
| chr10 | 102503746 | 102504769 |
| chr10 | 121974816 | 121975166 |
| chr10 | 127016787 | 127017101 |
| chr10 | 130300872 | 130301711 |
| chr10 | 130300873 | 130301818 |
| chr10 | 130300877 | 130301822 |
| chr10 | 130301216 | 130301710 |
| chr10 | 131176467 | 131177158 |
| chr10 | 132364373 | 132364686 |
| chr11 | 50411955 | 50412827 |
| chr11 | 64500855 | 64501064 |
| chr11 | 67507943 | 67508641 |
| chr11 | 70371335 | 70371506 |
| chr11 | 101741684 | 101862965 |
| chr11 | 113934810 | 113935454 |
| chr12 | 7018519 | 7019024 |
| chr12 | 72187438 | 121352789 |
| chr12 | 108363619 | 108363988 |
| chr13 | 26972766 | 26973546 |
| chr13 | 41405577 | 41431247 |
| chr13 | 113176451 | 113176543 |
| chr13 | 113672393 | 113672556 |
| chr14 | 54396756 | 54397539 |
| chr14 | 65791577 | 65791691 |
| chr14 | 105162457 | 105162664 |
| chr14 | 105162463 | 105162692 |
| chr15 | 20818350 | 21387486 |
| chr15 | 34437411 | 34583661 |
| chr15 | 34437413 | 34583663 |
| chr15 | 34437417 | 34583667 |
| chr15 | 34437419 | 34583669 |
| chr15 | 61317774 | 61318106 |
| chr15 | 77699271 | 77699769 |
| chr15 | 77699272 | 77699834 |
| chr15 | 77699372 | 77699798 |
| chr15 | 100146720 | 100152953 |
| chr15 | 101415240 | 101415431 |
| chr16 | 3263736 | 3264539 |
| chr16 | 11517543 | 11888071 |
| chr16 | 34586256 | 34587329 |
| chr16 | 48932277 | 48932703 |
| chr16 | 75206880 | 75212516 |
| chr16 | 89566206 | 89566313 |
| chr17 | 7314901 | 43900458 |
| chr17 | 80024304 | 80024653 |
| chr17 | 80497949 | 80498109 |
| chr17 | 81416011 | 81416564 |
| chr19 | 1015734 | 1015998 |
| chr19 | 1082083 | 1082826 |
| chr19 | 3556867 | 3556992 |
| chr19 | 33249731 | 33250382 |
| chr19 | 49149355 | 49150430 |
| chr2 | 3046153 | 3046312 |
| chr2 | 11618093 | 11618226 |
| chr2 | 15047555 | 15047669 |
| chr2 | 32916230 | 65130711 |
| chr2 | 32916242 | 61471084 |
| chr2 | 32916246 | 47906689 |
| chr2 | 32916249 | 64751321 |
| chr2 | 112054511 | 112054760 |
| chr2 | 130797566 | 130798206 |
| chr2 | 131673078 | 131673984 |
| chr2 | 131673107 | 131674013 |
| chr2 | 184473608 | 184473719 |
| chr2 | 217924298 | 230067556 |
| chr2 | 238098568 | 238098659 |
| chr20 | 2379056 | 2379968 |
| chr20 | 10323922 | 10324068 |
| chr20 | 25601604 | 25601733 |
| chr20 | 29754977 | 30503718 |
| chr20 | 30503373 | 30503590 |
| chr20 | 31067989 | 31072348 |
| chr20 | 40687694 | 40688803 |
| chr20 | 43696475 | 43697012 |
| chr20 | 58695014 | 58695334 |
| chr20 | 58695051 | 58695340 |
| chr20 | 64078178 | 64078281 |
| chr21 | 41179065 | 41179572 |
| chr21 | 43792716 | 43827775 |
| chr22 | 22183015 | 22183156 |
| chr22 | 38286051 | 38522454 |
| chr22 | 49973632 | 49973802 |
| chr22 | 49973721 | 49973805 |
| chr3 | 4492336 | 4493280 |
| chr3 | 40309582 | 40310226 |
| chr3 | 57536920 | 57538314 |
| chr3 | 57536923 | 57538401 |
| chr3 | 58646722 | 58647883 |
| chr3 | 93470362 | 93470781 |
| chr3 | 93470366 | 93470787 |
| chr3 | 93470372 | 93470788 |
| chr3 | 95749272 | 95752155 |
| chr3 | 122680622 | 122681276 |
| chr3 | 187139152 | 187139665 |
| chr3 | 194943264 | 194943687 |
| chr4 | 679157 | 679270 |
| chr4 | 1808681 | 1810082 |
| chr4 | 8781387 | 8781864 |
| chr4 | 37686340 | 53393790 |
| chr4 | 49657558 | 49657703 |
| chr5 | 761866 | 762155 |
| chr5 | 1229908 | 1230139 |
| chr5 | 17712709 | 17715744 |
| chr5 | 49660052 | 49661065 |
| chr5 | 107820286 | 107926854 |
| chr5 | 134495854 | 178660760 |
| chr5 | 139452273 | 139452656 |
| chr5 | 139452276 | 139453769 |
| chr5 | 144132924 | 144135488 |
| chr5 | 162906578 | 162906821 |
| chr5 | 178585408 | 178585741 |
| chr5 | 178585545 | 178585710 |
| chr5 | 178585545 | 178585725 |
| chr5 | 178585563 | 178585740 |
| chr5 | 178585576 | 178585725 |
| chr5 | 178941268 | 178942055 |
| chr5 | 179858344 | 179858897 |
| chr6 | 1053742 | 1054031 |
| chr6 | 12012670 | 40674678 |
| chr6 | 34072812 | 34073139 |
| chr6 | 35786784 | 35799011 |
| chr6 | 44044809 | 44045014 |
| chr6 | 166381920 | 166383126 |
| chr6 | 168270042 | 168270257 |
| chr6 | 170029659 | 170030109 |
| chr7 | 467302 | 467445 |
| chr7 | 482868 | 483641 |
| chr7 | 5038077 | 5218556 |
| chr7 | 66728586 | 73967789 |
| chr7 | 66728718 | 66728824 |
| chr7 | 70855001 | 70900090 |
| chr7 | 73831397 | 73832436 |
| chr7 | 76498794 | 76998017 |
| chr7 | 98085806 | 98086636 |
| chr7 | 100952735 | 100954962 |
| chr7 | 100957483 | 100958895 |
| chr7 | 100957644 | 100958273 |
| chr7 | 100958069 | 100958902 |
| chr7 | 100967866 | 100967982 |
| chr8 | 6648888 | 6649329 |
| chr8 | 8385665 | 8386470 |
| chr8 | 22694374 | 22695375 |
| chr8 | 81029288 | 81029908 |
| chr8 | 97775153 | 97775956 |
| chr8 | 138691547 | 138692217 |
| chr8 | 138691772 | 138692274 |
| chr8 | 140457841 | 140458165 |
| chr8 | 144413370 | 144414442 |
| chr9 | 34681484 | 34681981 |
| chr9 | 109523566 | 109524282 |
| chr9 | 121464631 | 121465541 |
| chr9 | 133459570 | 133460026 |
| chr9 | 134182936 | 134183082 |
| chr9 | 135726800 | 135727090 |
| chrX | 118975462 | 118976315 |
| chrY | 10691091 | 11296015 |
| chrY | 10747321 | 56833371 |
| chrY | 10747447 | 56833370 |
| chrY | 10754890 | 56834835 |
| chrY | 10757508 | 10807352 |
| chrY | 10808562 | 56834414 |
| chrY | 10808593 | 11294557 |
| chrY | 10945179 | 11295108 |
| chrY | 10986390 | 11295108 |
| chrY | 11019390 | 56832930 |
| chrY | 11295516 | 11295812 |

TABLE 7B

OVCAR8 Circle-Co-ordinate

| Chromosome | Start | End |
|---|---|---|
| chr1 | 820254 | 821729 |
| chr1 | 5991759 | 5992492 |
| chr1 | 16049926 | 16059816 |
| chr1 | 16049943 | 16059833 |
| chr1 | 67685301 | 67686335 |
| chr1 | 68111973 | 68113082 |
| chr1 | 115337875 | 115338240 |
| chr1 | 118100880 | 118101547 |
| chr1 | 146035461 | 146036230 |
| chr1 | 169109067 | 169116581 |
| chr1 | 183635163 | 183636009 |
| chr1 | 231337501 | 231338302 |
| chr10 | 1327203 | 1328008 |
| chr10 | 1327752 | 1327998 |
| chr10 | 3171472 | 3172728 |
| chr10 | 5889307 | 5890241 |
| chr10 | 73493290 | 73902830 |
| chr10 | 73625154 | 73625998 |
| chr10 | 103457332 | 103528085 |
| chr10 | 125824767 | 125825185 |
| chr10 | 133379291 | 133380329 |
| chr11 | 32091028 | 32091715 |
| chr11 | 36361949 | 36362944 |
| chr11 | 58900904 | 59058535 |
| chr11 | 65111820 | 65112583 |
| chr11 | 65111854 | 65112617 |
| chr11 | 65454428 | 65455299 |
| chr11 | 94768278 | 94769012 |
| chr11 | 96046281 | 96049860 |
| chr11 | 131680550 | 131680810 |
| chr12 | 49188504 | 49189169 |
| chr12 | 72187438 | 121352801 |
| chr12 | 72187438 | 121352828 |
| chr12 | 106031789 | 107832773 |
| chr12 | 132394117 | 132394264 |
| chr12 | 132767162 | 132768482 |
| chr13 | 99979566 | 99980600 |
| chr14 | 22944939 | 68530452 |
| chr14 | 34993060 | 34994348 |
| chr14 | 89582374 | 89651359 |
| chr14 | 99682866 | 99684031 |
| chr15 | 34437415 | 34583665 |
| chr15 | 34437417 | 34583667 |
| chr15 | 52956597 | 52962902 |
| chr15 | 73368967 | 73370349 |
| chr16 | 180349 | 181511 |
| chr16 | 556017 | 556749 |
| chr16 | 727065 | 728199 |
| chr16 | 2897471 | 2938600 |
| chr16 | 3263738 | 3264553 |
| chr16 | 4537445 | 4538545 |
| chr16 | 11515716 | 11515802 |
| chr16 | 19167930 | 19168994 |
| chr16 | 85524999 | 85525924 |
| chr16 | 87778023 | 87779287 |
| chr16 | 88864107 | 88864190 |
| chr17 | 2393037 | 2393792 |
| chr17 | 2548168 | 2548359 |
| chr17 | 2719771 | 2719870 |
| chr17 | 4949070 | 4950160 |
| chr17 | 21512856 | 21513067 |
| chr17 | 48489520 | 48495417 |
| chr17 | 73311110 | 73312131 |
| chr17 | 82022150 | 82023088 |
| chr18 | 2655698 | 2656500 |
| chr18 | 23585472 | 23586444 |
| chr18 | 78865825 | 78865986 |
| chr19 | 2247066 | 2247173 |
| chr19 | 5804123 | 5804956 |
| chr19 | 38361959 | 38362711 |
| chr19 | 43533437 | 43534093 |
| chr19 | 50717378 | 50718792 |
| chr19 | 50965118 | 50979784 |
| chr2 | 15275199 | 15698869 |
| chr2 | 26344760 | 26346102 |
| chr2 | 32916239 | 61694863 |
| chr2 | 32916240 | 47906682 |
| chr2 | 32916240 | 62705935 |
| chr2 | 32916241 | 47906684 |
| chr2 | 32916242 | 47906659 |
| chr2 | 32916242 | 47906684 |
| chr2 | 32916242 | 65130709 |
| chr2 | 32916242 | 65130722 |
| chr2 | 32916243 | 47906659 |
| chr2 | 32916243 | 47906684 |
| chr2 | 32916243 | 65130696 |
| chr2 | 32916243 | 65130723 |
| chr2 | 32916244 | 65130723 |
| chr2 | 32916249 | 47906652 |
| chr2 | 32916249 | 47906692 |
| chr2 | 32916249 | 63050778 |
| chr2 | 32916251 | 63050764 |
| chr2 | 32916485 | 68252667 |
| chr2 | 32916486 | 68252644 |
| chr2 | 64751429 | 64752103 |
| chr2 | 95345680 | 95346417 |
| chr2 | 112054511 | 112054755 |
| chr2 | 112054511 | 112054760 |
| chr2 | 112054517 | 112054766 |
| chr2 | 112054521 | 112054770 |
| chr2 | 112054540 | 112054789 |
| chr2 | 120344562 | 120345151 |
| chr2 | 130797589 | 130798201 |
| chr2 | 131673097 | 131674003 |
| chr2 | 149770397 | 149770867 |
| chr2 | 217924298 | 230067554 |
| chr2 | 232316045 | 232317104 |
| chr2 | 238136072 | 238170279 |
| chr2 | 240565954 | 240566669 |
| chr20 | 4686300 | 4687201 |
| chr20 | 47390643 | 47392362 |
| chr20 | 50058565 | 50059626 |
| chr20 | 62321196 | 62321566 |
| chr20 | 63626131 | 63626529 |
| chr20 | 63864274 | 63864949 |
| chr21 | 36698450 | 36699153 |
| chr21 | 41641805 | 41645951 |
| chr21 | 44276504 | 44287965 |
| chr22 | 41022258 | 41099935 |
| chr22 | 49954522 | 50077839 |
| chr22 | 50526222 | 50527277 |
| chr3 | 4492336 | 4493280 |
| chr3 | 39151021 | 39151883 |
| chr3 | 49903154 | 49903959 |
| chr3 | 62102209 | 62134661 |
| chr3 | 93470432 | 93470782 |
| chr3 | 136196102 | 136196691 |
| chr3 | 185841649 | 185842697 |
| chr3 | 186107709 | 186109107 |
| chr3 | 187908289 | 188155866 |
| chr4 | 8409618 | 8409856 |
| chr4 | 123200629 | 123237107 |
| chr5 | 72308111 | 72308619 |
| chr5 | 75672824 | 75672958 |
| chr5 | 141556735 | 141557945 |
| chr5 | 172869377 | 172870408 |
| chr5 | 180027197 | 180027869 |
| chr5 | 180292079 | 180293203 |
| chr6 | 10412434 | 10413454 |
| chr6 | 54059859 | 54063910 |
| chr6 | 73520186 | 73521216 |
| chr7 | 1940927 | 1941012 |
| chr7 | 65038261 | 65873269 |
| chr7 | 65038264 | 65873256 |
| chr7 | 74224754 | 74266926 |
| chr7 | 96953101 | 96953331 |
| chr7 | 96953107 | 96953337 |
| chr7 | 116525211 | 116526304 |
| chr7 | 155347769 | 155348946 |
| chr8 | 6648888 | 6649329 |
| chr8 | 10358133 | 10358945 |
| chr8 | 43776203 | 43780756 |
| chr8 | 46232819 | 46233833 |
| chr8 | 46326736 | 46327963 |
| chr8 | 97775153 | 97775956 |
| chr8 | 106950568 | 140950071 |
| chr8 | 124172009 | 127735305 |
| chr8 | 132926653 | 132931277 |
| chr8 | 142470915 | 142472027 |
| chr8 | 142675202 | 142676313 |
| chr8 | 143936732 | 143937961 |
| chr9 | 29101300 | 29212824 |
| chr9 | 91711805 | 91711994 |
| chr9 | 124300966 | 124301913 |
| chr9 | 124475741 | 124513868 |
| chr9 | 128702455 | 128703411 |
| chr9 | 128702509 | 128703378 |
| chr9 | 136841161 | 136842352 |
| chr9 | 137085966 | 137086993 |
| chrX | 23776133 | 23789070 |
| chrX | 109626770 | 111410864 |

TABLE 8A

GBM-Circle-Co-ordinate

| Chromosome | Start | End |
|---|---|---|
| chr1 | 64966005 | 64966496 |
| chr1 | 84504918 | 84506819 |
| chr1 | 84583988 | 84584807 |
| chr1 | 145766785 | 180632057 |
| chr1 | 151196520 | 174925904 |
| chr1 | 151196522 | 174925904 |
| chr1 | 153725238 | 184792551 |
| chr1 | 154405472 | 163071873 |
| chr1 | 154845372 | 184386914 |
| chr1 | 154972744 | 154974193 |
| chr1 | 155001928 | 155002212 |
| chr1 | 155060293 | 155061294 |
| chr1 | 155127314 | 155127819 |
| chr1 | 155255048 | 155255453 |
| chr1 | 156049807 | 156050565 |
| chr1 | 156668047 | 180911938 |
| chr1 | 156669826 | 156671068 |
| chr1 | 156751557 | 156752007 |
| chr1 | 160205363 | 160205955 |
| chr1 | 161193281 | 161194054 |
| chr1 | 161973884 | 161974485 |
| chr1 | 166973577 | 185045404 |
| chr1 | 172042525 | 172043191 |
| chr1 | 179229435 | 179230029 |
| chr1 | 180581079 | 180582589 |
| chr1 | 181033847 | 211402298 |
| chr1 | 182390614 | 182391699 |
| chr1 | 182782444 | 182783301 |
| chr1 | 183023187 | 183023614 |
| chr1 | 183635172 | 183635927 |
| chr1 | 183982735 | 183984414 |
| chr1 | 184387029 | 184387764 |
| chr1 | 197274106 | 197275027 |
| chr1 | 200487879 | 200670105 |
| chr1 | 200487881 | 200670105 |
| chr1 | 200519011 | 216682930 |
| chr1 | 200738793 | 200739587 |
| chr1 | 201829091 | 201829574 |
| chr1 | 201885750 | 201886886 |
| chr1 | 201888629 | 201888930 |
| chr1 | 202624523 | 202624815 |
| chr1 | 203762608 | 205210542 |
| chr1 | 203795861 | 203796446 |
| chr1 | 203918512 | 203920808 |
| chr1 | 204075317 | 204076435 |
| chr1 | 204088274 | 204089351 |
| chr1 | 204113681 | 204114651 |
| chr1 | 204195186 | 204195272 |
| chr1 | 204359455 | 204360458 |
| chr1 | 204693110 | 204693871 |
| chr1 | 204870665 | 204871285 |
| chr1 | 209217822 | 220644618 |
| chr1 | 212558445 | 212559077 |
| chr1 | 218164711 | 218165595 |
| chr1 | 221742469 | 221742697 |
| chr1 | 224132159 | 224132788 |
| chr1 | 228082971 | 228083950 |
| chr1 | 228166243 | 228166863 |
| chr1 | 230792727 | 230793627 |
| chr1 | 236142439 | 236143373 |
| chr1 | 239805359 | 239806557 |
| chr1 | 244834480 | 244835214 |
| chr1 | 244835295 | 244835819 |
| chr1 | 244863672 | 244864439 |
| chr1 | 244863744 | 244864625 |
| chr1 | 247112014 | 247112822 |
| chr10 | 598527 | 21522293 |
| chr10 | 688838 | 689479 |
| chr10 | 780276 | 1156914 |
| chr10 | 3838638 | 3838840 |
| chr10 | 4556448 | 4556820 |
| chr10 | 6055951 | 6750243 |
| chr10 | 6145120 | 6145826 |
| chr10 | 7526614 | 7526796 |
| chr10 | 11178077 | 11178772 |
| chr10 | 12780179 | 12780565 |
| chr10 | 13398035 | 13398444 |
| chr10 | 13759218 | 13759711 |
| chr12 | 1051503 | 18850669 |
| chr12 | 1629491 | 1630454 |
| chr12 | 1936233 | 1937034 |
| chr12 | 2269978 | 2271177 |
| chr12 | 2485455 | 49869926 |
| chr12 | 3218682 | 3219921 |
| chr12 | 3290825 | 3291611 |
| chr12 | 4271222 | 34163846 |
| chr12 | 4275095 | 4275562 |
| chr12 | 4661540 | 4926117 |
| chr12 | 4809311 | 4809579 |
| chr12 | 4909301 | 4909961 |
| chr12 | 5375582 | 7848472 |
| chr12 | 6085714 | 22046642 |
| chr12 | 6200410 | 6201491 |
| chr12 | 6310554 | 6310838 |
| chr12 | 6340972 | 6342141 |
| chr12 | 6451582 | 6452683 |
| chr12 | 6470758 | 6471073 |
| chr12 | 6493271 | 6493921 |
| chr12 | 6532407 | 6532987 |
| chr12 | 6541709 | 6542424 |
| chr12 | 6555369 | 6556134 |
| chr12 | 6612918 | 9065165 |
| chr12 | 6689008 | 6689534 |
| chr12 | 6872549 | 6873498 |
| chr12 | 6872561 | 6873630 |
| chr12 | 6872570 | 6873414 |
| chr12 | 6872609 | 6873364 |
| chr12 | 6943981 | 6944747 |
| chr12 | 7017979 | 7018629 |
| chr12 | 9406057 | 9406815 |
| chr12 | 11106587 | 11141418 |
| chr12 | 11171204 | 11171665 |
| chr12 | 11193559 | 21401648 |
| chr12 | 13140151 | 13140423 |
| chr12 | 13989044 | 31119083 |
| chr12 | 14677102 | 14677517 |
| chr12 | 16028932 | 28312310 |
| chr12 | 16069154 | 16069535 |
| chr12 | 17309333 | 51365826 |
| chr12 | 18853138 | 38427804 |
| chr12 | 19573695 | 19574034 |
| chr12 | 19997967 | 19998683 |
| chr12 | 22312201 | 43083671 |
| chr12 | 22479040 | 46245879 |
| chr12 | 23557106 | 23557457 |
| chr12 | 24074602 | 43435680 |
| chr12 | 26294989 | 26295205 |
| chr12 | 27003157 | 27003358 |
| chr12 | 27780252 | 27780503 |
| chr12 | 28229211 | 42511702 |
| chr12 | 30985473 | 30986124 |
| chr12 | 31035894 | 31036710 |
| chr12 | 31253062 | 31253426 |
| chr12 | 31325580 | 31326390 |
| chr12 | 32679300 | 32679800 |
| chr12 | 57520779 | 57738877 |
| chr12 | 57613669 | 57849420 |
| chr12 | 57626258 | 57627487 |
| chr12 | 57743299 | 57744699 |
| chr12 | 57752087 | 57752409 |
| chr12 | 57845558 | 57846484 |
| chr12 | 57845752 | 57846204 |
| chr12 | 57846369 | 57847180 |
| chr12 | 57846463 | 57847047 |
| chr12 | 57846607 | 57847165 |
| chr12 | 57846622 | 57847163 |
| chr12 | 64183437 | 64201818 |
| chr12 | 64205591 | 64205832 |
| chr12 | 68808057 | 68808821 |
| chr12 | 68808072 | 68808663 |
| chr12 | 68808084 | 68808848 |
| chr12 | 68808088 | 68808852 |
| chr12 | 68854394 | 68854717 |
| chr12 | 70243203 | 70243710 |
| chr12 | 70339104 | 70340286 |
| chr12 | 70366131 | 70366634 |
| chr13 | 98142478 | 98143018 |
| chr14 | 21069214 | 21070150 |
| chr16 | 17069197 | 17069872 |
| chr2 | 119759550 | 119760206 |
| chr2 | 120243329 | 120244353 |
| chr2 | 120323288 | 120324584 |
| chr2 | 120343530 | 120344263 |
| chr2 | 120874171 | 120874482 |
| chr2 | 121284318 | 121285387 |
| chr2 | 121736950 | 121737144 |
| chr20 | 50587642 | 50588296 |
| chr20 | 62763506 | 62764448 |
| chr21 | 36156615 | 36157286 |
| chr3 | 1380642 | 16916542 |
| chr3 | 1381023 | 18524644 |
| chr3 | 2005622 | 9749637 |
| chr3 | 2055313 | 24426507 |
| chr3 | 2114929 | 12975877 |
| chr3 | 2653647 | 26661140 |
| chr3 | 3836140 | 3836924 |
| chr3 | 4451973 | 13005987 |
| chr3 | 4492394 | 4493265 |
| chr3 | 4610793 | 9344482 |
| chr3 | 4978519 | 4979468 |
| chr3 | 4978882 | 4979293 |
| chr3 | 4979481 | 4980525 |
| chr3 | 4979483 | 4980521 |
| chr3 | 4979483 | 4980527 |
| chr3 | 7611126 | 7612684 |
| chr3 | 9396910 | 9397426 |
| chr3 | 9397090 | 9397599 |
| chr3 | 9397105 | 9397600 |
| chr3 | 9664961 | 15227004 |
| chr3 | 9729591 | 9731053 |
| chr3 | 9731442 | 9732193 |
| chr3 | 9731464 | 9732452 |
| chr3 | 9750451 | 10244242 |
| chr3 | 9769731 | 9770698 |
| chr3 | 9867287 | 35069346 |
| chr3 | 9900901 | 12659483 |
| chr3 | 10011673 | 24519362 |
| chr3 | 10192254 | 10192829 |
| chr3 | 11558344 | 38758954 |
| chr3 | 12695080 | 31891110 |
| chr3 | 12877258 | 12878051 |
| chr3 | 14505385 | 14506337 |
| chr3 | 14952240 | 14953093 |
| chr3 | 14989937 | 32728446 |
| chr3 | 15070016 | 15070414 |
| chr3 | 15859863 | 15860748 |
| chr3 | 16838193 | 32432716 |
| chr3 | 23643946 | 23644017 |
| chr3 | 24690698 | 24690891 |
| chr3 | 24727339 | 33441283 |
| chr3 | 30120884 | 30121481 |
| chr3 | 31532404 | 31533455 |
| chr3 | 31532573 | 31533170 |
| chr3 | 38024871 | 38025569 |
| chr5 | 38424982 | 38425801 |
| chr5 | 112876503 | 113477413 |
| chr5 | 112921709 | 112922234 |
| chr6 | 170553341 | 170554228 |
| chr6 | 170553353 | 170554229 |
| chr7 | 566477 | 3120272 |
| chr7 | 758064 | 1504364 |
| chr7 | 816235 | 5423957 |
| chr7 | 842893 | 844114 |
| chr7 | 877149 | 877905 |
| chr7 | 1675652 | 1676485 |
| chr7 | 1863141 | 1863832 |
| chr7 | 2051611 | 2051865 |
| chr7 | 2108812 | 2109408 |
| chr7 | 2518869 | 2519545 |
| chr7 | 2555139 | 2555791 |
| chr7 | 2555160 | 2555801 |
| chr7 | 2824330 | 2824981 |
| chr7 | 4568973 | 22645954 |
| chr7 | 5189908 | 5190401 |
| chr7 | 5314533 | 32640868 |
| chr7 | 5419279 | 5419771 |
| chr7 | 5422649 | 5423318 |
| chr7 | 5423213 | 5423719 |
| chr7 | 5423262 | 5423997 |
| chr7 | 5423619 | 15097133 |
| chr7 | 5424177 | 5424561 |
| chr7 | 5426567 | 5427418 |
| chr7 | 5426767 | 5427458 |
| chr7 | 5427958 | 5428420 |
| chr7 | 5495052 | 5495870 |
| chr7 | 5529611 | 5529881 |
| chr7 | 5555319 | 5556379 |
| chr7 | 5562478 | 5562829 |
| chr7 | 5562868 | 5563736 |
| chr7 | 5940928 | 24290822 |
| chr7 | 6348164 | 6348830 |
| chr7 | 6663591 | 6664181 |
| chr7 | 7182565 | 7183366 |
| chr7 | 7182583 | 7183371 |
| chr7 | 7182583 | 7183379 |
| chr7 | 7366485 | 7366813 |
| chr7 | 7968806 | 7969213 |
| chr7 | 8211746 | 8212824 |
| chr7 | 12686862 | 12687366 |
| chr7 | 15685536 | 15686608 |
| chr7 | 16172291 | 16172842 |
| chr7 | 16645029 | 16646144 |
| chr7 | 16645029 | 16646148 |
| chr7 | 16791061 | 45161749 |
| chr7 | 17939839 | 17940206 |
| chr7 | 20785002 | 20785994 |
| chr7 | 22068591 | 28214477 |
| chr7 | 23112529 | 52333389 |
| chr7 | 23467960 | 23468957 |
| chr7 | 23531548 | 23532064 |
| chr7 | 23597308 | 23597875 |
| chr7 | 23597313 | 23597804 |
| chr7 | 24979521 | 24980211 |
| chr7 | 25358811 | 25359654 |
| chr7 | 26199862 | 26200872 |
| chr7 | 26200157 | 26200758 |
| chr7 | 26201589 | 26202167 |
| chr7 | 26201688 | 26202187 |
| chr7 | 26201726 | 26202319 |
| chr7 | 26398232 | 26399115 |
| chr7 | 26398593 | 26399104 |
| chr7 | 27932817 | 27933015 |
| chr7 | 28093785 | 28094825 |
| chr7 | 28728202 | 28729424 |
| chr7 | 28955818 | 28956631 |
| chr7 | 28957165 | 28957688 |
| chr7 | 29684991 | 29685594 |
| chr7 | 29807241 | 29808597 |
| chr7 | 29923911 | 38032345 |
| chr7 | 30134699 | 30134912 |
| chr7 | 30284567 | 30285288 |
| chr7 | 30403177 | 63833641 |
| chr7 | 30859768 | 30860226 |
| chr7 | 30970589 | 30971398 |
| chr7 | 30988565 | 30989242 |
| chr7 | 31028953 | 31029537 |
| chr7 | 32495247 | 32495425 |
| chr7 | 32495247 | 44290368 |
| chr7 | 32495339 | 75637995 |
| chr7 | 32495387 | 32495854 |
| chr7 | 32495526 | 32496384 |
| chr7 | 32891299 | 32891736 |
| chr7 | 32891332 | 32891664 |
| chr7 | 32891376 | 32891725 |
| chr7 | 32891463 | 32891863 |
| chr7 | 32940937 | 32941877 |
| chr7 | 33106283 | 66690235 |
| chr7 | 33905330 | 75753251 |
| chr7 | 36152917 | 36153180 |
| chr7 | 37447873 | 37448658 |
| chr7 | 37448461 | 37449012 |
| chr7 | 37694432 | 37695074 |
| chr7 | 38630872 | 38631407 |
| chr7 | 40134689 | 40134993 |
| chr7 | 42882780 | 42883423 |
| chr7 | 43113002 | 43113288 |
| chr7 | 43302658 | 43303286 |
| chr7 | 43621564 | 73584455 |
| chr7 | 44104256 | 44104976 |
| chr7 | 44258666 | 44259720 |
| chr7 | 44283601 | 44284382 |
| chr7 | 44748481 | 44748778 |
| chr7 | 44759879 | 44760721 |
| chr7 | 44847897 | 44848215 |
| chr7 | 45221571 | 45222265 |
| chr7 | 46445954 | 47791231 |
| chr7 | 47498274 | 47498915 |
| chr7 | 47669120 | 47669934 |
| chr7 | 48089080 | 48089762 |
| chr7 | 48089085 | 48089687 |
| chr7 | 48089088 | 48089687 |
| chr7 | 51315667 | 51316488 |
| chr7 | 54540504 | 55427933 |
| chr7 | 54545917 | 69728178 |
| chr7 | 54545918 | 69728179 |
| chr7 | 54545919 | 69728180 |
| chr7 | 54545920 | 69728181 |
| chr7 | 54546741 | 95435111 |
| chr7 | 54559198 | 85891526 |
| chr7 | 54574602 | 54574764 |
| chr7 | 54664661 | 54665262 |
| chr7 | 54719572 | 54719673 |
| chr7 | 54749370 | 54749455 |
| chr7 | 54759297 | 54759989 |
| chr7 | 54772112 | 54772260 |
| chr7 | 54838829 | 54839962 |
| chr7 | 54842242 | 55144987 |
| chr7 | 54926047 | 54926501 |
| chr7 | 54934716 | 54934948 |
| chr7 | 54990188 | 54990444 |
| chr7 | 55019041 | 55019493 |
| chr7 | 55019425 | 55020289 |
| chr7 | 55019432 | 55020296 |
| chr7 | 55065796 | 63833642 |
| chr7 | 55079061 | 55079886 |
| chr7 | 55079340 | 55079543 |
| chr7 | 55096317 | 55096604 |
| chr7 | 55099112 | 55100172 |
| chr7 | 55121709 | 55121815 |
| chr7 | 55204534 | 55204714 |
| chr7 | 55204599 | 55205038 |
| chr7 | 55248874 | 103726868 |
| chr7 | 55254946 | 55255061 |
| chr7 | 55357825 | 55358406 |
| chr7 | 55365588 | 55366305 |
| chr7 | 55365882 | 55366595 |
| chr7 | 55365931 | 55366287 |
| chr7 | 55462619 | 100121157 |
| chr7 | 55476188 | 55476717 |
| chr7 | 55522676 | 55523553 |
| chr7 | 55887679 | 55887846 |
| chr7 | 55911573 | 55911992 |
| chr7 | 57217223 | 57218360 |
| chr7 | 57377436 | 57378568 |
| chr7 | 62306503 | 62307437 |
| chr7 | 63887444 | 63887972 |
| chr7 | 64375052 | 64376339 |
| chr7 | 64666513 | 64667690 |
| chr7 | 64876998 | 64877594 |
| chr7 | 65038263 | 65873256 |
| chr7 | 65872920 | 106101470 |
| chr7 | 66557799 | 66558602 |
| chr7 | 66804821 | 66805770 |
| chr7 | 66869598 | 102121355 |
| chr7 | 68423209 | 68423933 |
| chr7 | 68517194 | 68518274 |
| chr7 | 68677651 | 68678666 |
| chr7 | 69597442 | 69598054 |
| chr7 | 70789656 | 70790299 |
| chr7 | 71289453 | 71290476 |
| chr7 | 71617312 | 77654871 |
| chr7 | 72536159 | 74975729 |
| chr7 | 73521985 | 73522415 |
| chr7 | 73521992 | 73522796 |
| chr7 | 73578686 | 73578791 |
| chr7 | 73578687 | 73578976 |
| chr7 | 73578694 | 73578983 |
| chr7 | 73578748 | 73578915 |
| chr7 | 73578771 | 73578964 |
| chr7 | 73578776 | 73578952 |
| chr7 | 73842536 | 73842691 |
| chr7 | 74026955 | 74027774 |
| chr7 | 74289392 | 74290297 |
| chr7 | 74302637 | 74303194 |
| chr7 | 74422001 | 74422802 |
| chr7 | 74561334 | 101816538 |
| chr7 | 74658175 | 74658548 |
| chr7 | 75444254 | 123535082 |
| chr7 | 75992296 | 75992692 |
| chr7 | 76294304 | 76295059 |
| chr7 | 76358986 | 76359299 |
| chr7 | 77537713 | 77538438 |
| chr7 | 77537723 | 77538438 |
| chr7 | 77588904 | 116328871 |
| chr7 | 77696392 | 77697132 |
| chr7 | 81853877 | 106101475 |
| chr7 | 82443567 | 82443852 |
| chr7 | 87152373 | 87153302 |
| chr7 | 87380245 | 87380395 |
| chr7 | 87768643 | 87768844 |
| chr7 | 90595890 | 90596258 |
| chr7 | 92081964 | 92084396 |
| chr7 | 92836599 | 92837103 |
| chr7 | 94340036 | 120262455 |
| chr7 | 94634392 | 94634500 |
| chr7 | 94908687 | 107169546 |
| chr7 | 95751905 | 95752261 |
| chr7 | 98088714 | 98088906 |
| chr7 | 98281258 | 98281695 |
| chr7 | 98400467 | 98401208 |
| chr7 | 98548211 | 98548642 |
| chr7 | 99142564 | 99143110 |
| chr7 | 99877866 | 148369220 |
| chr7 | 100081857 | 100082693 |
| chr7 | 100101489 | 130784620 |
| chr7 | 100468315 | 100469342 |
| chr7 | 100468331 | 100469398 |
| chr7 | 100587964 | 100588171 |
| chr7 | 100675497 | 100676030 |
| chr7 | 100705393 | 100705992 |
| chr7 | 100866346 | 100867206 |
| chr7 | 100895014 | 100895426 |
| chr7 | 101121857 | 117337557 |
| chr7 | 101217022 | 101217660 |
| chr7 | 101244454 | 129311994 |
| chr7 | 101858629 | 125702323 |
| chr7 | 105012691 | 105013444 |
| chr7 | 105012765 | 105013599 |
| chr7 | 105388499 | 105388955 |
| chr7 | 106176892 | 106178314 |
| chr7 | 106660459 | 106661008 |
| chr7 | 107126552 | 107127207 |
| chr7 | 107168928 | 107169747 |
| chr7 | 107169128 | 107169574 |
| chr7 | 107186271 | 145657553 |
| chr7 | 107743642 | 107744135 |
| chr7 | 109153735 | 158599032 |
| chr7 | 109346908 | 138903813 |
| chr7 | 112428974 | 112430025 |
| chr7 | 112939041 | 112940148 |
| chr7 | 116354371 | 116355362 |
| chr7 | 116862495 | 116862972 |
| chr7 | 117289680 | 117290731 |
| chr7 | 117732730 | 117733101 |
| chr7 | 121050433 | 125702330 |
| chr7 | 121390856 | 130441327 |
| chr7 | 121396009 | 121396443 |
| chr7 | 121485754 | 121486229 |
| chr7 | 121798039 | 125702326 |
| chr7 | 121825548 | 135504503 |
| chr7 | 121872967 | 121873770 |
| chr7 | 121873145 | 121873685 |
| chr7 | 122007955 | 122008215 |
| chr7 | 122143434 | 122144109 |
| chr7 | 122315074 | 122315852 |
| chr7 | 122422454 | 128770627 |
| chr7 | 123534689 | 123535138 |
| chr7 | 123534750 | 123535260 |
| chr7 | 123534755 | 123535059 |
| chr7 | 123534843 | 123535240 |
| chr7 | 123714643 | 123716017 |
| chr7 | 123748665 | 123749070 |
| chr7 | 124032255 | 124032938 |
| chr7 | 124939324 | 138460175 |
| chr7 | 127392315 | 127392825 |
| chr7 | 127651045 | 127651928 |
| chr7 | 127800020 | 127800741 |
| chr7 | 127903724 | 127904867 |
| chr7 | 128830311 | 128831046 |
| chr7 | 128830313 | 128831038 |
| chr7 | 128836207 | 128837171 |
| chr7 | 128938079 | 128938889 |
| chr7 | 129210368 | 129210929 |
| chr7 | 129825579 | 129826004 |
| chr7 | 129952727 | 129953080 |
| chr7 | 131449613 | 137207190 |
| chr7 | 131620083 | 131621066 |
| chr7 | 134458931 | 134459339 |
| chr7 | 134740335 | 134742448 |
| chr7 | 135147705 | 135148595 |
| chr7 | 135170484 | 135170768 |
| chr7 | 135430518 | 135431214 |
| chr7 | 135508978 | 135509869 |
| chr7 | 135662425 | 135662736 |
| chr7 | 135662441 | 135662961 |
| chr7 | 135662454 | 135662962 |
| chr7 | 138001666 | 138002157 |
| chr7 | 138460328 | 138460974 |
| chr7 | 139359071 | 139359698 |
| chr7 | 139359743 | 139360454 |
| chr7 | 139359747 | 139360458 |
| chr7 | 139359776 | 139360528 |
| chr7 | 139359788 | 139360534 |
| chr7 | 140696710 | 140697173 |
| chr7 | 141073471 | 141074117 |
| chr7 | 141824552 | 141825288 |
| chr7 | 143062564 | 143063056 |
| chr7 | 143288138 | 143288708 |
| chr7 | 143381736 | 143382434 |
| chr7 | 143883721 | 143885562 |
| chr7 | 145264055 | 145264395 |
| chr7 | 148550876 | 148552123 |
| chr7 | 148699203 | 148699817 |
| chr7 | 148869198 | 148869792 |
| chr7 | 148884707 | 148884923 |
| chr7 | 149090889 | 149091542 |
| chr7 | 149126066 | 149126864 |
| chr7 | 149126408 | 149126872 |
| chr7 | 149262041 | 149262481 |
| chr7 | 150378525 | 150379249 |
| chr7 | 150378535 | 150379238 |
| chr7 | 150404928 | 150405365 |
| chr7 | 150799961 | 150800560 |
| chr7 | 150800045 | 150800765 |
| chr7 | 151058236 | 151059510 |
| chr7 | 151080866 | 151081399 |
| chr7 | 151086835 | 151087474 |
| chr7 | 151794821 | 151795362 |
| chr7 | 151876086 | 151876757 |
| chr7 | 151876202 | 151876600 |
| chr7 | 152392257 | 152392614 |
| chr7 | 153748481 | 153749090 |
| chr7 | 153886922 | 153887320 |
| chr7 | 155207901 | 155208612 |
| chr7 | 155408388 | 155408755 |
| chr7 | 155456329 | 155457416 |
| chr7 | 155456466 | 155457437 |
| chr7 | 155482110 | 155482685 |
| chr7 | 155644024 | 155644650 |
| chr7 | 155996333 | 155997001 |
| chr7 | 156892725 | 156893279 |
| chr7 | 156892783 | 156893280 |
| chr7 | 157336503 | 157337226 |
| chr7 | 157480457 | 157480524 |
| chr7 | 157776206 | 157776878 |
| chr7 | 157942369 | 157943054 |
| chr7 | 158337001 | 158337867 |
| chr7 | 158387515 | 158387575 |
| chr7 | 158389650 | 158390273 |

TABLE 8B

LGG-Circle-Co-ordinate

| Chromosome | Start | End |
|---|---|---|
| chr10 | 451780 | 452902 |
| chr10 | 622240 | 623211 |
| chr10 | 627326 | 628393 |
| chr10 | 931481 | 931774 |
| chr10 | 1048924 | 1049215 |
| chr10 | 2791713 | 2792118 |
| chr10 | 2909221 | 2909421 |
| chr10 | 7526685 | 7526946 |
| chr10 | 10364731 | 10364943 |
| chr10 | 12042910 | 12043368 |
| chr10 | 13468136 | 13468347 |
| chr10 | 14009518 | 14010158 |
| chr10 | 15638355 | 15638557 |
| chr10 | 17462254 | 17462602 |
| chr10 | 23105352 | 23105566 |
| chr10 | 27972102 | 27972318 |
| chr10 | 28866078 | 28866498 |
| chr10 | 35167435 | 35167649 |
| chr10 | 35217923 | 35218479 |
| chr10 | 35399613 | 35399817 |
| chr11 | 27718480 | 27719071 |
| chr11 | 28824878 | 36215494 |
| chr11 | 29133708 | 29430833 |
| chr11 | 30361399 | 30361786 |
| chr11 | 31877072 | 36188811 |
| chr11 | 34051927 | 34089285 |
| chr11 | 36412110 | 36417948 |
| chr11 | 40757804 | 40758177 |
| chr11 | 41020557 | 41020792 |
| chr11 | 41993131 | 41993746 |
| chr12 | 1220611 | 1220987 |
| chr12 | 1887173 | 1887629 |
| chr12 | 2566230 | 2567218 |
| chr12 | 2655959 | 2656384 |
| chr12 | 3873162 | 3873501 |
| chr12 | 4269455 | 4269935 |
| chr12 | 4808968 | 4809346 |
| chr12 | 6201406 | 6202487 |
| chr12 | 14416728 | 14417905 |
| chr12 | 14416733 | 14417910 |
| chr12 | 22334674 | 22335314 |
| chr12 | 23950012 | 23951025 |
| chr12 | 24550846 | 24551366 |
| chr12 | 24562189 | 24562739 |
| chr12 | 25250528 | 25250832 |
| chr12 | 25385573 | 25386229 |
| chr12 | 25590144 | 25591139 |
| chr12 | 26122251 | 26122607 |
| chr12 | 26126211 | 26126673 |
| chr12 | 26318709 | 26319740 |
| chr12 | 123269376 | 123269728 |
| chr12 | 124564923 | 124565634 |
| chr12 | 124633178 | 124633548 |
| chr12 | 124704862 | 124705613 |
| chr12 | 124876311 | 124876701 |
| chr13 | 31161065 | 31162109 |
| chr13 | 31161760 | 31162411 |
| chr13 | 32171921 | 32172085 |
| chr13 | 33312426 | 33313993 |
| chr13 | 39602796 | 39603394 |
| chr13 | 42373132 | 42373518 |
| chr13 | 43357313 | 43358580 |
| chr13 | 43787270 | 43787806 |
| chr13 | 44905256 | 44906942 |
| chr13 | 45340620 | 45341216 |
| chr15 | 77544262 | 77544752 |
| chr15 | 77631823 | 77632422 |
| chr15 | 77658254 | 78304292 |
| chr2 | 15801996 | 15802226 |
| chr2 | 15948390 | 15948633 |
| chr2 | 16052063 | 16452027 |
| chr2 | 16177696 | 17516120 |
| chr2 | 16203188 | 16314758 |
| chr2 | 16225124 | 16226720 |
| chr2 | 16392024 | 16392729 |
| chr2 | 16567034 | 17412029 |
| chr2 | 17051859 | 17051967 |
| chr2 | 17192282 | 17192652 |
| chr2 | 17279980 | 17334701 |
| chr2 | 17296668 | 17297551 |
| chr2 | 17584356 | 17877776 |
| chr20 | 21101875 | 21103066 |
| chr20 | 21102035 | 21103071 |
| chr20 | 21507504 | 21508584 |
| chr20 | 21510732 | 21511606 |
| chr20 | 21559646 | 21560756 |
| chr20 | 61331561 | 61331713 |
| chr20 | 61636004 | 61636722 |
| chr20 | 62302171 | 62302425 |
| chr20 | 62727606 | 62728174 |
| chr20 | 62753728 | 62754298 |
| chr20 | 62950883 | 62952025 |
| chr21 | 44763786 | 44764391 |
| chr22 | 17158717 | 17159102 |
| chr22 | 17370270 | 17370659 |
| chr22 | 17556835 | 17557856 |
| chr22 | 17948634 | 17948855 |
| chr22 | 19122051 | 19122480 |
| chr22 | 19397634 | 19398790 |
| chr22 | 19431813 | 19432298 |
| chr22 | 19431816 | 19432690 |
| chr22 | 19447504 | 19447908 |
| chr22 | 19456815 | 29346704 |
| chr22 | 19714547 | 19714863 |
| chr22 | 19892999 | 19894058 |
| chr22 | 20120953 | 20121786 |
| chr22 | 20438002 | 20439089 |
| chr22 | 21924540 | 21925240 |
| chr22 | 21959628 | 21960328 |
| chr22 | 22294833 | 22295899 |
| chr22 | 22629577 | 23309299 |
| chr22 | 23070251 | 50323770 |
| chr22 | 23117779 | 23118223 |
| chr22 | 23217793 | 23219197 |
| chr22 | 23289419 | 23290788 |
| chr22 | 23894378 | 23895015 |
| chr22 | 24155288 | 24155986 |
| chr22 | 24554935 | 24555855 |
| chr22 | 24588479 | 24588978 |
| chr22 | 24599340 | 50185758 |
| chr22 | 27918865 | 27919304 |
| chr22 | 29312278 | 29312617 |
| chr22 | 29633586 | 29634532 |
| chr22 | 29703122 | 29703716 |
| chr22 | 30278275 | 30279505 |
| chr22 | 30305822 | 30307111 |
| chr22 | 30322022 | 30322713 |
| chr22 | 30423106 | 30423870 |
| chr22 | 31160519 | 31161285 |
| chr22 | 32994230 | 32995146 |
| chr22 | 35350568 | 35350924 |
| chr22 | 36386364 | 36387078 |
| chr22 | 36765056 | 36766037 |
| chr22 | 37546198 | 37547135 |
| chr22 | 37639118 | 37639707 |
| chr22 | 37802494 | 37803082 |
| chr22 | 37903524 | 37904732 |
| chr22 | 37933195 | 37933868 |
| chr22 | 38172230 | 38173202 |
| chr22 | 38316214 | 38316905 |
| chr22 | 39520512 | 39521118 |
| chr22 | 40636219 | 40636743 |
| chr22 | 41362208 | 41363117 |
| chr22 | 41381981 | 41382237 |
| chr22 | 41446894 | 41447916 |
| chr22 | 41620416 | 41621041 |
| chr22 | 41951103 | 41951997 |
| chr22 | 43219896 | 43220416 |
| chr22 | 44072194 | 44073291 |
| chr22 | 45268219 | 45268688 |
| chr22 | 45671838 | 45672262 |
| chr22 | 45963749 | 45965072 |
| chr22 | 46006482 | 46007088 |
| chr22 | 46008209 | 46009545 |
| chr22 | 46051158 | 46052466 |
| chr22 | 46053600 | 46054209 |
| chr22 | 46092576 | 46093100 |
| chr22 | 46114165 | 46115257 |
| chr22 | 46121998 | 46122669 |
| chr22 | 46223878 | 46224577 |
| chr22 | 46267500 | 46267910 |
| chr22 | 48639086 | 48639692 |
| chr4 | 42590721 | 42590957 |
| chr4 | 53347797 | 53347952 |
| chr4 | 53377579 | 53378044 |
| chr4 | 53377580 | 53378040 |
| chr4 | 53377585 | 53378050 |
| chr4 | 53814347 | 53814952 |
| chr4 | 53907355 | 53908790 |
| chr4 | 54064464 | 54064629 |
| chr4 | 54091962 | 54092343 |
| chr4 | 54106070 | 54294081 |
| chr4 | 54149233 | 54363317 |
| chr4 | 54222805 | 54224049 |
| chr4 | 54226675 | 54227227 |
| chr4 | 54227466 | 54227931 |
| chr4 | 54227786 | 54228468 |
| chr4 | 54229320 | 54230209 |
| chr4 | 54229770 | 54231241 |
| chr4 | 54229943 | 54230726 |
| chr4 | 54230225 | 54230722 |
| chr4 | 54230229 | 54230721 |
| chr4 | 54230262 | 54230917 |
| chr4 | 54230921 | 54231996 |
| chr4 | 54230960 | 54231953 |
| chr4 | 54230971 | 54231909 |
| chr4 | 54231000 | 54231661 |
| chr4 | 54231419 | 54233246 |
| chr4 | 54232225 | 54233168 |
| chr4 | 54232950 | 54233688 |
| chr4 | 54233178 | 54233596 |
| chr4 | 54233313 | 54233539 |
| chr4 | 54233328 | 54234186 |
| chr4 | 54233433 | 54233943 |
| chr4 | 54233436 | 54233953 |
| chr4 | 54233514 | 54234308 |
| chr4 | 54233526 | 54234320 |
| chr4 | 54233528 | 54234306 |
| chr4 | 121485093 | 121485837 |
| chr4 | 121801326 | 121801877 |
| chr6 | 1963574 | 1963787 |
| chr6 | 2765552 | 2766281 |
| chr6 | 2998547 | 2999693 |
| chr6 | 3068566 | 3069012 |
| chr6 | 3157702 | 3158207 |
| chr6 | 3226300 | 3226990 |
| chr6 | 3227946 | 3228352 |
| chr6 | 3258796 | 3259407 |
| chr6 | 3750189 | 3750672 |
| chr6 | 4135856 | 4136240 |
| chr6 | 4879536 | 31830200 |
| chr6 | 6006171 | 6006913 |
| chr6 | 7107766 | 7108289 |
| chr6 | 8589908 | 37705558 |
| chr6 | 11829807 | 11830447 |
| chr6 | 12011735 | 12012189 |
| chr6 | 13303056 | 13303955 |
| chr6 | 13328293 | 13328915 |
| chr6 | 13454836 | 13455845 |
| chr6 | 13455085 | 13455739 |
| chr6 | 13486750 | 13487272 |
| chr6 | 17206414 | 28219012 |
| chr6 | 18939907 | 22260357 |
| chr6 | 19837326 | 19837856 |
| chr6 | 20401791 | 20402701 |
| chr6 | 20402570 | 20403370 |
| chr6 | 20402771 | 20403536 |
| chr6 | 25234798 | 25235009 |
| chr6 | 26204047 | 26204509 |
| chr6 | 26215444 | 26216398 |
| chr6 | 26250017 | 26250540 |
| chr6 | 26596146 | 26596990 |
| chr6 | 27547704 | 27547777 |
| chr6 | 27791525 | 27792022 |
| chr6 | 37697031 | 37697396 |
| chr6 | 37698513 | 37699144 |
| chr6 | 45421718 | 45422060 |
| chr6 | 147507945 | 147508574 |
| chr7 | 1027417 | 1028170 |
| chr7 | 1028745 | 1028982 |
| chr7 | 1084226 | 1084908 |
| chr7 | 1887549 | 1887871 |
| chr7 | 3104818 | 3105037 |
| chr7 | 6196422 | 12703520 |
| chr7 | 7182582 | 7183375 |
| chr7 | 12633550 | 12633931 |
| chr7 | 18795239 | 47850679 |
| chr7 | 21057116 | 21057504 |
| chr7 | 21427239 | 21427909 |
| chr7 | 24366262 | 24366476 |
| chr7 | 25372256 | 25372508 |
| chr7 | 26922489 | 26922709 |
| chr7 | 29468136 | 75878934 |
| chr7 | 30202937 | 30203160 |
| chr7 | 37448569 | 37449073 |
| chr7 | 37594938 | 37595333 |
| chr7 | 43094291 | 43094676 |
| chr7 | 43198594 | 55095878 |
| chr7 | 43583079 | 43583681 |
| chr7 | 43583089 | 43583551 |
| chr7 | 47239505 | 47239700 |
| chr7 | 47581673 | 47581954 |
| chr7 | 53295842 | 102969981 |
| chr7 | 54985672 | 54986034 |
| chr7 | 66227004 | 100121157 |
| chr7 | 76998862 | 76999020 |
| chr7 | 77854449 | 77855208 |
| chr7 | 80303614 | 80304606 |
| chr7 | 80469719 | 80470103 |
| chr7 | 82522987 | 102010434 |
| chr7 | 83770949 | 83771664 |
| chr7 | 84603803 | 84701767 |
| chr7 | 89301767 | 89301845 |
| chr7 | 92134066 | 92134983 |
| chr7 | 92833009 | 92833307 |
| chr7 | 93707490 | 93707877 |
| chr7 | 95893641 | 95894013 |
| chr7 | 100678303 | 100678977 |
| chr7 | 101159943 | 151098370 |
| chr7 | 101789613 | 101790209 |
| chr7 | 102464887 | 102465368 |
| chr7 | 103295864 | 103297039 |
| chr7 | 104713587 | 104713934 |
| chr7 | 105792015 | 105792368 |
| chr7 | 107961212 | 107961600 |
| chr7 | 110933819 | 137072025 |
| chr7 | 111873996 | 111874269 |
| chr7 | 115144752 | 115145169 |
| chr7 | 115367187 | 115367624 |
| chr7 | 116674913 | 121917354 |
| chr7 | 116862628 | 140640933 |
| chr7 | 120274022 | 120274701 |
| chr7 | 120857619 | 135168315 |
| chr7 | 121899118 | 121899693 |
| chr7 | 122849986 | 122850376 |
| chr7 | 122886156 | 122886840 |
| chr7 | 128032229 | 128032582 |
| chr7 | 129156226 | 129594545 |
| chr7 | 132317781 | 132318497 |
| chr7 | 135144930 | 138169113 |
| chr7 | 137515860 | 137672617 |
| chr7 | 138891189 | 138891500 |
| chr7 | 139036196 | 139036796 |
| chr7 | 140696626 | 140697350 |
| chr7 | 149623685 | 149624410 |
| chr7 | 149881972 | 149882712 |
| chr7 | 150902493 | 150903084 |
| chr7 | 152829518 | 152830349 |
| chr7 | 152951425 | 152951618 |
| chr7 | 155482115 | 155482690 |
| chr7 | 156601658 | 156601859 |
| chr7 | 157676552 | 157677126 |
| chr7 | 157689623 | 157690069 |
| chr7 | 158387515 | 158387575 |
| chr8 | 47744742 | 47745338 |
| chr8 | 47865086 | 47865295 |
| chr8 | 80040901 | 80043224 |
| chr8 | 80578459 | 80578765 |
| chr8 | 94895218 | 94895737 |
| chr8 | 94895218 | 94895747 |
| chr8 | 94895255 | 94895731 |
| chr8 | 94951639 | 94952202 |
| chr8 | 95133566 | 95134143 |
| chr8 | 96161482 | 96162547 |
| chr8 | 97644502 | 97645005 |
| chr8 | 100244374 | 100245036 |
| chr8 | 100721642 | 100722256 |
| chr8 | 100950593 | 100951363 |
| chr8 | 102239040 | 102239619 |
| chr8 | 102410735 | 102411574 |
| chr8 | 102654515 | 102655665 |
| chr8 | 102654770 | 102655674 |
| chr8 | 102655733 | 102656138 |
| chr8 | 102806185 | 102806779 |
| chr8 | 103499902 | 103500296 |
| chr8 | 103500801 | 103501474 |
| chr8 | 108977521 | 108978688 |
| chr8 | 109333663 | 109334253 |
| chr8 | 109539825 | 109540079 |
| chr8 | 114456278 | 114457554 |
| chr8 | 115536468 | 115537073 |
| chr8 | 115930683 | 142497631 |
| chr8 | 117121187 | 117122030 |
| chr8 | 122861063 | 122862899 |
| chr8 | 123273404 | 123274098 |
| chr8 | 123273854 | 123274736 |
| chr8 | 123274336 | 123274746 |
| chr8 | 123768036 | 123768383 |
| chr8 | 123949572 | 131282269 |
| chr8 | 124728160 | 124728489 |
| chr8 | 124915882 | 124916823 |
| chr8 | 125334526 | 125335491 |
| chr8 | 125432345 | 125433200 |
| chr8 | 126558355 | 126558639 |
| chr8 | 127736221 | 127736859 |
| chr8 | 127737027 | 127737842 |
| chr8 | 129918116 | 129918703 |
| chr8 | 131648667 | 131649912 |
| chr8 | 132079147 | 132080026 |
| chr9 | 14007444 | 14314053 |
| chr9 | 14303680 | 14304490 |
| chr9 | 16710713 | 16711151 |
| chr9 | 16993492 | 16993728 |

TABLE 9

Co-ordinates of circle identified (Junction tag >1) in GBM cell lines

| Chromosome | Start | End |
|---|---|---|
| chr1 | 32500550 | 32539743 |
| chr1 | 32500565 | 32539742 |
| chr1 | 235078245 | 235078548 |
| chr10 | 130300872 | 130301711 |
| chr11 | 3257642 | 3303607 |
| chr11 | 69917483 | 69917610 |
| chr11 | 70371325 | 70371556 |
| chr12 | 130043305 | 130043472 |
| chr13 | 101820638 | 101820755 |
| chr14 | 39033513 | 39033641 |
| chr17 | 41632758 | 41633279 |
| chr17 | 80024304 | 80024653 |
| chr17 | 80497980 | 80498098 |
| chr17 | 82898940 | 82899120 |
| chr18 | 9809076 | 9809266 |
| chr19 | 310547 | 310663 |
| chr19 | 310597 | 310837 |
| chr19 | 5761463 | 5761618 |
| chr19 | 49867633 | 49867701 |
| chr2 | 3046153 | 3046312 |
| chr2 | 55048772 | 55204950 |
| chr2 | 216766042 | 216766166 |
| chr20 | 31060443 | 31064213 |
| chr20 | 63158461 | 63158602 |
| chr20 | 63626185 | 63626527 |
| chr21 | 37806440 | 37806837 |
| chr21 | 46645209 | 46645315 |
| chr22 | 47549004 | 47549110 |
| chr22 | 49973631 | 49973803 |
| chr3 | 93470432 | 93470782 |
| chr3 | 93470451 | 93470644 |
| chr3 | 194822580 | 194825701 |
| chr4 | 41216413 | 86934989 |
| chr4 | 59802691 | 59803100 |
| chr4 | 118591708 | 119454712 |
| chr4 | 133845222 | 133845431 |
| chr5 | 117973459 | 117973635 |
| chr6 | 18155080 | 18155232 |
| chr6 | 18155085 | 18155223 |
| chr6 | 44044545 | 44045272 |
| chr6 | 54059859 | 54063910 |
| chr7 | 47669121 | 47669934 |
| chr7 | 54590796 | 55256528 |
| chr7 | 54771165 | 54782815 |
| chr7 | 65038261 | 65873269 |
| chr7 | 65038264 | 65873256 |
| chr7 | 76498794 | 76998017 |
| chr7 | 76498805 | 76998018 |
| chr7 | 98547916 | 98548526 |
| chr7 | 157480227 | 157480519 |
| chr8 | 138691547 | 138692217 |
| chr9 | 89254202 | 96157888 |
| chr9 | 127802760 | 127802926 |
| chr9 | 134376587 | 134376652 |
| chrX | 7515541 | 7515675 |
| chrX | 153382567 | 153383002 |
| chrY | 10945179 | 11295108 |
| chrY | 10962886 | 11294341 |

### Discussion of Examples 1-8

It is demonstrated herein that the application of the Circle_finder algorithm (see, e.g., Table 11) to ATAC-seq data can identify eccDNA in cell lines and tissues. Most of the eccDNA thus identified in the cell line could be detected by inverse PCR on DNA enriched for extrachromosomal DNA with disenrichment of linear DNA fragments. The metaphase spreads from OVCAR8 cells showed the presence of these eccDNA loci as a signal off the chromosomes, consistent with the loci being extrachromosomal. Even if ATAC-seq is performed without experimentally dis-enriching linear chromosomal DNA and/or enriching circular DNA, this approach is useful to identify loci that are either contained in eccDNAs or have suffered a tandem segmental duplication in the chromosome. The identification of eccDNA/duplication in the EGFR locus in GBM cell lines as well as GBMs suggested that existing ATAC-seq data from other cancers should also be examined closely to find the driver gene amplification on eccDNA/duplication events in each tumor type. Indeed, several cancer driver genes were found located in such loci (Table 3). These results suggest that deeper sequencing of tumors by ATAC-seq with longer paired-end-reads will identify many more clinically important sites involved in eccDNA/duplication in these tumors.

Chromosome ends are protected by telomeres. Once the chromosome suffers a catastrophic fragmentation, as in chromothripsis, some parts of the chromosome may be protected from degradation by eccDNA formation. EccDNA can also be generated from extra linear DNA produced by some kind of copying mechanism as a byproduct of DNA replication or repair. Either way, the present results show that eccDNA are very prevalent in cancer cell lines and tumors, and that ATAC-seq is an effective method to identify such eccDNA.

It has been reported that eccDNA longer than a few kb may have origins of replication and may get amplified independent of the main chromosome. Thus, if an eccDNA harbors an oncogene, then amplification of such eccDNA in tumor cells will increase the fitness of the tumor cell. In addition, since a centromere is absent in the eccDNA (Turner et al., Nature 543, 122-125 (2017)), eccDNA may segregate unevenly between daughter cells and result in tumor heterogeneity (deCarvalho et al., Nat Genet 50, 708-717 (2018)). Both these mechanisms will increase the likelihood that if a particular type of therapy inhibits a gene resident on a pre-existing eccDNA, then the tumor is likely to acquire resistance through the selective amplification of that eccDNA.

In this context it is particularly exciting that circle (or gene-duplication) at an important locus in a subset of the tumor cells is identified by ATAC-seq even before the amplification is apparent by a CNV analysis of the whole tumor (FIG. 4D). To estimate whether ATAC-seq can identify loci in early, somatically mosaic states of amplification as eccDNA/segmental duplication, the amplicons identified by TCGA from gene array hybridization were analyzed. It is apparent that an amplicon has to be at least around 1.5 MB long to be detected as a single copy amplification by gene microarrays (3 copies per cell) (Materials and Methods for Examples 1-8 and FIG. 7). In contrast, somatically mosaic increase in copy number of loci far smaller than that length was detected by use of Circle_finder on ATAC-seq or WGS data. For example, Circle_finder on ATAC-seq data identifies sites of incipient amplification of the EGFR gene in a subset of tumor cells even before such amplification is detected by copy number measurements, predicting that even if the tumor responds to anti-EGF therapy, it is likely to recur because of amplification of the EGFR gene.

Many of the abundant eccDNA loci intersect with unprocessed pseudogenes, which are known to have introns and regulatory sequences, but crippled by stop codons in the open reading frames (Tutar, Comp Funct Genomics 2012, 424526 (2012)). Since eccDNA evolve and pick up substitution, insertion and deletion mutations (Turner et al., Nature 543, 122-125 (2017); Xu et al., Acta Neuropathol 137, 123-137 (2019)), it is tempting to speculate that amplification of unprocessed pseudogenes on eccDNA and their evolution may make these genes translationally competent to give an unknown advantage during tumorigenesis.

Finally, it is noted that a large fraction of eccDNA identified by ATAC-seq have properties similar to the microDNA reported earlier: length <1 kb, with peaks at 180 and 380 bases, high GC content, enrichment of their sites of origin in regions upstream of genes and in CpG islands and the presence of short sequences of homology flanking the chromosomal locus giving rise to the circle. The small size of these circles has thus been confirmed by rolling circle amplification (Shibata et al., Science 336, 82-86 (2012)), electron microscopy (Shibata et al., Science 336, 82-86 (2012)) and now by ATAC-seq, ruling out any possibility that the previously reported small size was due to preferential amplification of small circles. Although eccDNA longer than 2 kb were observed in mouse somatic tissue but the majority (>90%) of eccDNA were shorter than 2 kb (Dillon et al., Cell Rep 11, 1749-1759 (2015); Shibata et al., Science 336, 82-86 (2012)). Turner et al. (Turner et al., Nature 543, 122-125 (2017)) & deCarvalho et al. (deCarvalho et al., Nat Genet 50, 708-717 (2018)) have identified long circles of DNA in cancers, called ecDNA. It is believed that the long circles identified in tumors by ATAC-Seq, e.g. the one containing the EGFR gene, belong to this latter class of circles. The consistent properties of the small circles suggest that common mechanisms are involved in their generation in cell lines and in tumors, although it is unclear whether exactly the same mechanisms are involved in producing the longer circles seen in ecDNAs that give rise to clinically significant gene amplifications.

### Materials and Methods for Examples 1-8

ATAC-seq library preparation. ATAC-seq for cell lines was performed as per the OMNI-ATAC-seq protocol (Corces et al., Nat Methods 14, 959-962 (2017)). Briefly, C4-2 and OVCAR8 cells were grown in RPMI-1640 (Corning #10-040) supplemented with 10% FBS to about 80% confluence. 50,000 viable cells were lysed in 10 mM Tris-HCl (pH 7.4), 10 mM NaCl, 3 mM MgCl2 and 0.1% Tween-20. Nuclear pellet was then subjected to transposition reaction using Nextera DNA Sample Preparation kit (Illumina #FC-121-1030) in the presence of 0.01% digitonin and 0.1% Tween-20 at 37° C. for 30 minutes and cleaned up with DNA Clean and Concentrator-5 Kit (Zymo #D4014). For qPCR, 3 to 6 additional cycles of PCR amplification was performed using NEBNext High-Fidelity 2X PCR Master Mix (NEB #M0541L) and Nextera Index Kit (Illumina #15055289). Cleaned up libraries were quantified and pooled for sequencing by Novogene.

Identification of eccDNA from ATAC-seq and WGS libraries. Paired end reads were mapped to the hg38 genome build using bwa-mem (Li & Durbin, Bioinformatics 25,1754-1760 (2009)) with default setting. The split reads (reads not mapped in contiguous manner) were collected using tool samblaster ( Faust & Hall, Bioinformatics 30, 2503-2505 (2014)). If one tag of a paired read is mapped contiguously (one entry in mapped file) and other tag is mapped in a split manner (two entries in mapped file) then the particular read id will have three entries in alignment file. Therefore, all the read pair IDs that mapped to three unique sites in the genome from the alignment file were collected. Next, split reads that mapped uniquely at two positions on the same chromosome and in the same orientation were collected. Returning to the list of paired end IDs that mapped uniquely to three sites in the genome, paired end IDs were identified where the contiguously mapped read is between the two split reads and on the opposite strand. From this list a circle is annotated if at least one junctional sequence was found. For karyotype and box plot at least two junctional reads were considered.

Copy number amplification (CNA) Analysis. For each identified eccDNA (JTGE2) an upstream and downstream genomic interval of equivalent length was created. Next, the number of reads that mapped to each of the three intervals (upstream, eccDNA and downstream) were counted. Finally, CNA was computed by counting the number of mapped read in eccDNA interval divided by mean of the number of reads in upstream and downstream intervals. CNA value more than 1 would suggest the amplification of the locus defined by the eccDNA.

EccDNA isolation. EccDNA for FIG. 3 was prepared from the human cancer cell lines. The cells were grown on 150 mm plates until reaching confluence. Approximately 4e7 cells were isolated per sample. The cells were trypsinized and then spun down at 300 g. The cells were washed with PBS, spun down at 300 g and resuspended in 6 mL of re-suspension buffer (P1) of the Qiagen HiSpeed Plasmid MidiKit (catalog 12643). 6 mL lysis buffer (P2) was added according to manufacturer’s instructions. The cells were lysed for 5 minutes and before adding the neutralization buffer (P3), and incubation at room temperature for 10 minutes. The cell lysate was passed through the QIAfilter cartridge. 4 mL of equilibration buffer (QBT) was added to the HiSpeed Tip and allowed to pass through the resin. The lysate was added to the HiSpeed Tip and then the HiSpeed Tip was washed with 20 mL of washing buffer (QC). Then the DNA was eluted from the HiSpeed Tip with 5 mL of elution buffer (QF) and precipitated with 3.5 mL of isopropanol and incubation at room temperature for 5 minutes. The DNA was passed through a QIAprecipitator, which was washed with 2 mL of 70% ethanol. The excess ethanol was removed by passing air through the QIAprecipitator five times. The DNA was precipitated from the QIAprecipitator by 1 mL of TE buffer and quantified using a nanodrop spectrophotometer. The DNA eluted from the QIAprecipitator was then precipitated again by the addition of 2 mL of ethanol and 1 ug of glycogen and centrifugation at 15,000 g. The supernatant was removed, the DNA was air dried for 5 minutes, re-suspended in 20 uL of TE and warmed to 37° C. for 5 minutes. Then the DNA was digested with the Lucigen ATP-dependent plasmid safe DNase (catalog E3101K). The 10X buffer and ATP was added according to manufacturer’s recommendations. Additionally, RNAse A was added to the solution to digest RNA concurrently with the linear DNA. The sample was digested overnight and then purified using a Zymo PCR purification kit (catalog D4003). Briefly, the DNA binding buffer was added to the DNA solution in a 5:1 ratio. The mixture was then added to a Zymo-Spin column in a collection tube. The sample was centrifuged for 30 seconds at 10,000 g. Then the column was washed with 200 uL of DNA wash buffer and centrifuged for 30 seconds at 10,000 g. The wash step was repeated. The DNA was eluted by adding 50 uL of DNA elution buffer and centrifuging for 30 seconds at 10,000 g. The DNA was quantified by a nanodrop spectrophotometer to ensure digestion of the linear DNA and then the DNA digestion, purification, and quantification steps repeated until the DNA concentration no longer decreased after digestion. Together, this process helped ensure that the digestion of linear DNA was complete. These methods are comparable to the methods previously used where the loss of linear DNA was validated by quantifying the loss of linear DNA with QPCR compared to circular DNA. EM imaging in those experiments showed that the linear DNA was no longer present in the samples (Dillon et al., Cell Rep 11, 1749-1759 (2015); Shibata et al., Science 336, 82-86 (2012)).

Outward directed PCRs (inverse PCR) for detection of eccDNA. Outward directed primers were designed across the junctional tags identified from ATAC-seq analysis. PCR was done with Phusion High-Fidelity DNA polymerase (NEB) according to manufacturer’s instructions. 3 ng of purified circular DNA was used as template. Unless otherwise stated, all the computation and plots were made of eccDNA present on chr1-22, chrX & chrY.

Metaphase Fluorescence in-situ hybridization (Metaphase FISH). OVCAR8 cells were cultured in RPMI medium supplemented with 10% FBS and 1% penicillin/streptomycin in presence of 5% C02 in humidified incubator at 37° C. Cells were treated with 2 mM thymidine for 16 hours and released for 9 hours in regular medium followed by another block with 2 mM thymidine to arrest the cells at G1/S boundary. The cells were released from the double-thymidine block for 3 hours in regular medium and 9 hours in 0.1 µg/ml Colcemid. Mitotic cells were shaken off, washed twice with 1X PBS and resuspended in 75 mM KCl for 30 min at 370° C. The cells were centrifuged at 300 Xg for 5 min, fixed with Carnoy’s fixative (3:1 methanol:glacial acetic acid, v/v) on ice for 30 min, washed twice with fixative and metaphase spreads were prepared. The glass slides containing metaphase spreads were immersed in pre-warmed denaturation buffer (70% formamide, 2X SSC, pH 7.0) at 73° C. for 5 min and slides were serially dehydrated with ethanol (70%, 85%, 100%) for 2 min each and dried at room temperature until all the ethanol evaporated. The FISH probes (Empire Genomics) were denatured with hybridization buffer at 730° C. for 5 min and immediately chilled on ice for 2 min. The probe mixture was added onto the slide and coverslips were applied onto the slide and sealed with rubber cement and incubated at 370° C. for overnight in humidified chamber. The coverslips were removed and slides were washed with pre-warmed 0.4X SSC containing 0.3% NP-40 at 73° C. for 2 min followed by washing with 2X SCC buffer containing 0.1% NP-40 at room temperature for 5 min. The slides were dried at room and mounted with Vectashield DAPI medium.

List of TCGA ID that was used for LGG and GBM data analysis. LGG: TCGA-P5-A77X-01A, TCGA-DU-5870-02A, TCGA-DB-A75K-01A, TCGA-W9-A837-01A, TCGA-F6-A8O3-01A, TCGA-FG-A4MY-01A, TCGA-E1-A7YI-01A, TCGA-P5-A735-01A, TCGA-DU-6407-02B. GBM: TCGA-06-A7TK-01A, TCGA-4W-AA9S-01A, TCGA-OX-A56R-01A, TCGA-76-6656-01A, TCGA-RR-A6KB-01A, TCGA-06-A6S1-01A, TCGA-06-A5U0-01A, TCGA-06-A7TL-01A.

Testing the limit of detection of gene amplification by CNV measurements. It was tested whether the detection of eccDNAs from ATAC-Seq data can identify somatically mosaic amplifications before they can be detected by copy number variation analyses from genotyping array data. To determine the sensitivity of detection of an amplicon by genotyping arrays, the previously released copy number variation (CNV) results generated by the TCGA research network were downloaded. The algorithm used by the TCGA research network segments the chromosomes into smaller sections where an amplification or deletion is detected. Empirically, the resulting length of segments with CNV determined by the algorithm are the result of (1) the true length of the amplified or deleted segment and (2) the extent to which the segment was amplified or deleted. While one cannot know whether or not a reported CNV-segment should have been further segmented, it was hypothesized that if ten segments with a similar level of amplification were analyzed, the smallest length among them approximates the smallest length that can be detected by the algorithm at that level of amplification, since the power to detect CNV changes increases as the extent of amplification increases. The TCGA research network reported amplifications as segment mean >0, where segment mean is ln(Copy number/2). All segments with segment mean >0.1 were ordered by reported segment mean values. Bins of ten segments were analyzed for the smallest segment in each bin. The median segment mean value of each bin (extent of amplification) is plotted against and the log-transformed smallest segment length in that bin (FIG. 7).

The correlation between the segment length and segment amplification can be modeled as a linear function with the following formula:

ln(Minimum Segment Length) = 15.8304 - 2.7475*Median Segment Mean

This relatively simple model captured the relationship between minimum segment length and the extent of amplification as measured by segment mean (Adjusted R2 = 0.5442, p<2.2E-16).

If one extra copy of an amplicon is present in every single cell of the sample, the segment mean value is 0.585 [log2(3/2)]. From the linear model in FIG. 7, the minimum segment length detectable at this segment mean value is 1.5 MB. Therefore, most of the somatically mosaic amplifications driven by most of the eccDNAs in the present study (median length - about 2 kb) will not be captured using genotyping arrays.

The number of ATAC-seq libraries analyzed in this study for various tumor type as follow: ACC 8; BLCA 10; BRCA 70; CESC 3; CHOL 1; COAD 38; ESCA 17; GBM 8; HNSC 9; KIRC 15; KIRP 29; LGG 10; LIHC 15; LUAD 21; LUSC 12; MESO 5; PCPG 9; PRAD 21; SKCM 9; STAD 19; TGCT 8; THCA 12; UCEC 10.

### Example 9

### Methods of Detecting MicroDNA

MicroDNA are extrachromosomal circles of DNA seen in normal cells and in cancers (Paulsen et al., Trends Genet. 2018;34(4):270-8). Because microDNA are closed circular DNA molecules, they have to be linearized to produce DNA fragments that can be sequenced. This can be done by rolling circle amplification with random hexamers (Shibata et al., Science. 2012;336(6077):82-6) or by digestion with restriction enzymes. Tagmentation (FIG. 1A), where a Tn5 transposase is used to break linear genomic DNA and add a transposase sequence to the ends of the DNA fragments, is used commonly to probe which parts of the genome are epigenetically open in ATAC-seq (Buenrostro et al., Curr Protoc Mol Biol. 2015;109:21 9 1-9). Transposase tagging breaks DNA and adds tags to end of DNA. ATAC-seq can be used to identify open chromatin at genome level. It uses Tn5, a hyperactive transposase to insert sequencing adapters into open area of chromatin regions.

This Example demonstrates that tagmentation also breaks circles efficiently to produce linear DNA fragments that can be sequenced by high throughput sequencing to identify the junctional sequences that are diagnostic of circles (FIGS. 1A and 1B). Once linearized, the DNA is subjected to paired-end sequencing. An algorithm, such as Circle Finder (https://github.com/pk7zuva/Circle_finder; see also Table 11), can then be used to identify circles, based on criteria, such as those indicated in FIG. 1B and FIG. 1C. By way of elaboration but not limitation, mapped-unmapped pairs of reads are looked for, where one read maps uniquely to the genome, but the other end is unmappable to the genome because it does not exist in the native genome. The algorithm then examines whether the unmappable read can be mapped by splitting read in to two (the differently shaded parts in the figure), where the two parts of the split read map to the genome on the “opposite strand” as to the mapped read, and flanking the mapped read. Since the paired-read sequencing is performed on DNA fragments of a defined length (usually 300-400 base), it is also ensured that at least one of the parts of the split read is <400 bases from the mapped read.

In FIG. 1A, transposase tagging (diamonds) are schematically showing, breaking circles of DNA. Subsequent ligation of adaptors (shaded bars), amplification, and sequencing identifies the junctional sequences (shaded fused bars) unique to each circle. In FIG. 1B, it is shown that the paired end reads obtained from high throughput sequencing identify the junction that is unique to circular DNA (but cannot be mapped to genomic DNA), linked to a tag that is uniquely mapped to the genome, at a specific sequence, in a specific orientation and a specific distance from the junction. The algorithm first identifies the junction sequence as a split read mapping to the genome to identify a potential circle. The second step checks the perfectly mapped end (the other end of the paired end read) maps at a specified distance from at least one of the two halves of the junction sequence. Finally, the perfectly mapped end has to map to DNA that is flanked by the sequences that make up the junction sequence. In addition, our algorithm also checks the polarity of paired reads mapped read should be on one strand, and the split reads of the junction sequence on the opposite strand.

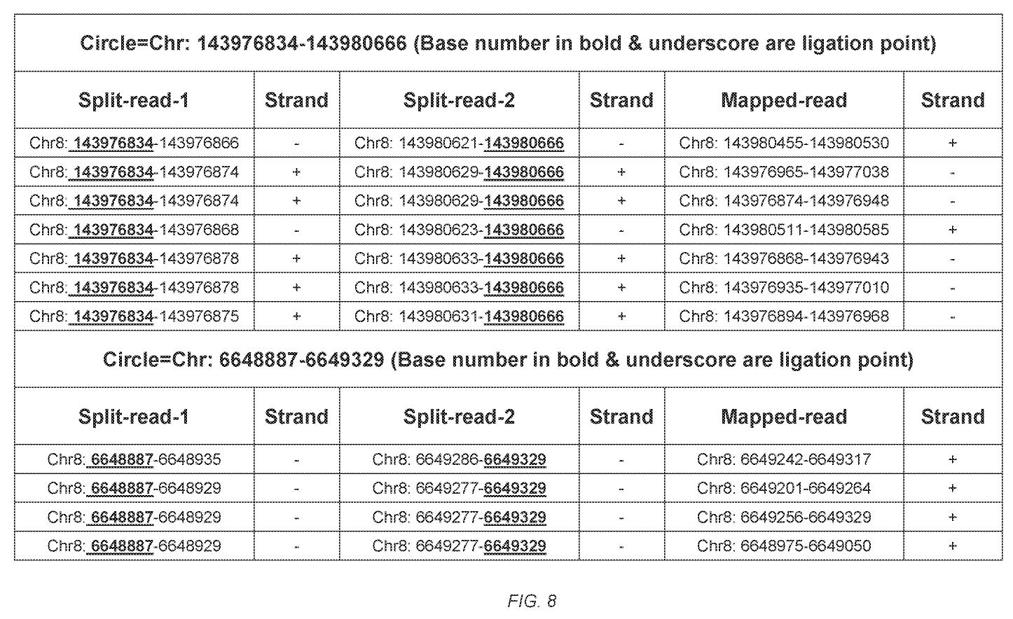

To demonstrate that this approach identifies microDNA, ATAC-seq was performed using a NEXTERA™ kit (Illumina), and data was analyzed looking for circles. Hundreds of circles with the typical size distribution of >90% being less than 500 bases were found, with two peaks at 200 and 500 bases (FIGS. 6 and 7). ATAC-seq data from single cells (Chen et al., Nat Commun. 2018;9(1):5345) was turned to next and microDNA in five different cell types from two species (FIG. 8 and Table 10) were identified.

TABLE 10

Total number of microDNA per Cell in Various Cell Types

| Sample Name | # of Single Cell | Total Number of Unique |
|---|---|---|
| | | microDNA |
| Human | | |
| Fibroblast | 373 | 394,933 |
| Human | | |
| K562 Lymphoblast | 312 | 481,168 |
| Mouse | | |
| Cardiomaocyte | 740 | 1,218,751 |
| CD4 | 741 | 650,363 |

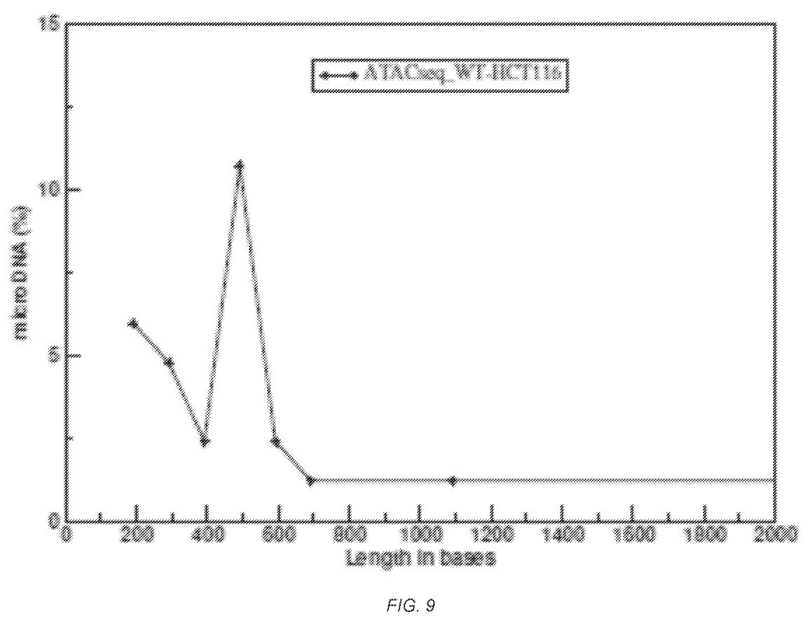

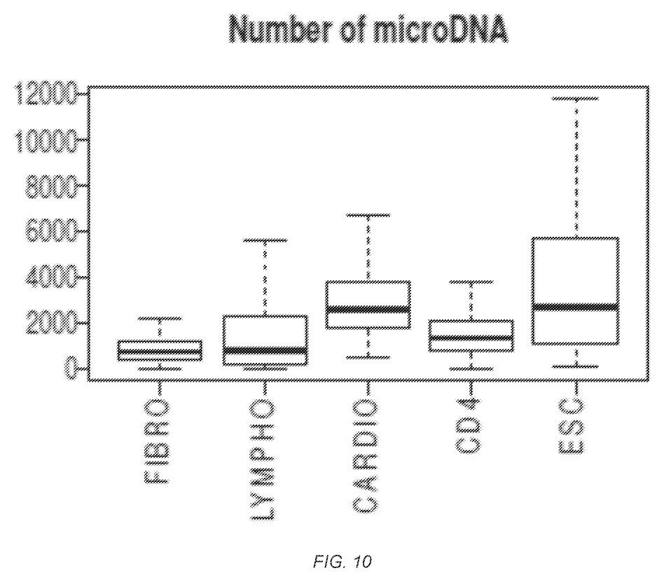

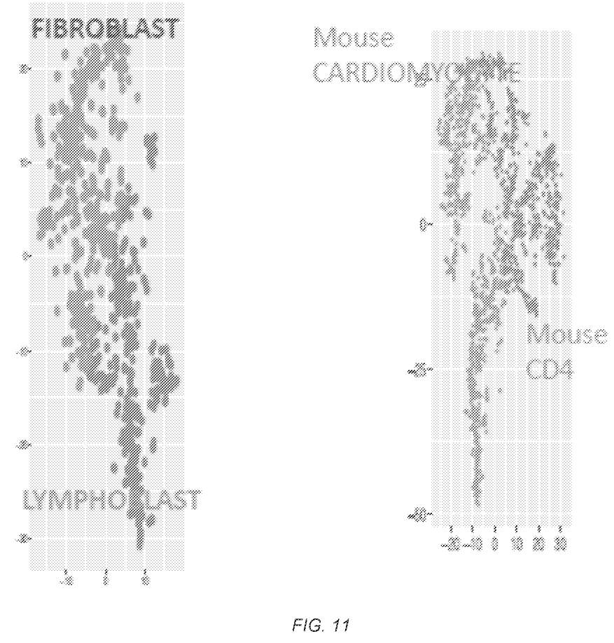

MicroDNA arise from epigenetically active parts of the genome, and since the epigenetically active parts of the genome are expected to be different in different cell types, it was hypothesized that the profile of the parts of the genome from which microDNA arise should be able to distinguish between cell types. TSNE plots (https://github.com/jkrijthe/Rtsne) of the microDNA genome-source profiles show that indeed the profiles can distinguish human fibroblasts from lymphoblasts or mouse cardiomyocytes from CD4+ T lymphocytes (FIG. 9).

In summary, standard tagmentation in ATAC-seq protocols can linearize circular DNA and identify microDNA in cell populations and in single cells. This simplifies the ability to identify microDNA and use the microDNA profiles to identify the tissue source of the microDNA for diagnosis. MicroDNA can be found as part of the circulating DNA in the blood. For example, circulating cell-free microDNA can be used for liquid biopsy as in screening of cancers and following the treatment response of cancers. MicroDNA circulating in the maternal blood can also be used for noninvasive prenatal testing of genetic disease in fetuses. Methods of detecting microDNA in accordance with the presently disclosed subject matter facilitate these applications.

### Example 10

### Screening Methods

Studies with eccDNAs on cancers in patients detect tumors poised to amplify a gene and acquire resistance. Also, methods for assessing circulating long eccDNAs in liquid biopsy provide for screening for cancers.

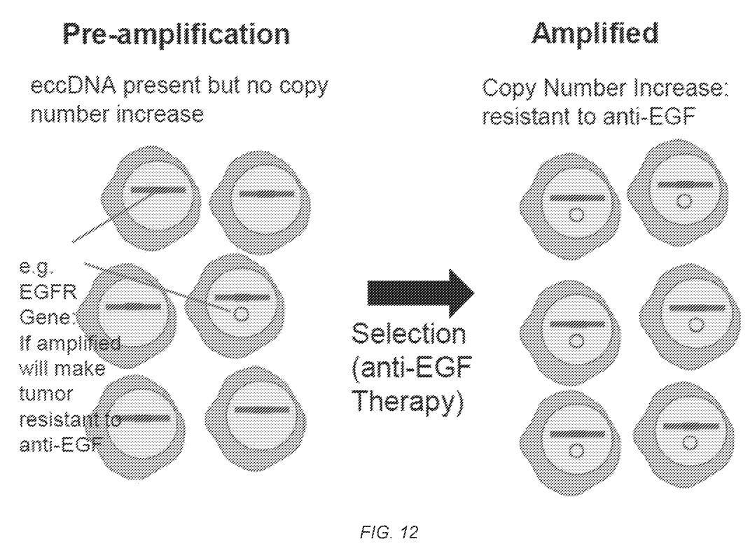

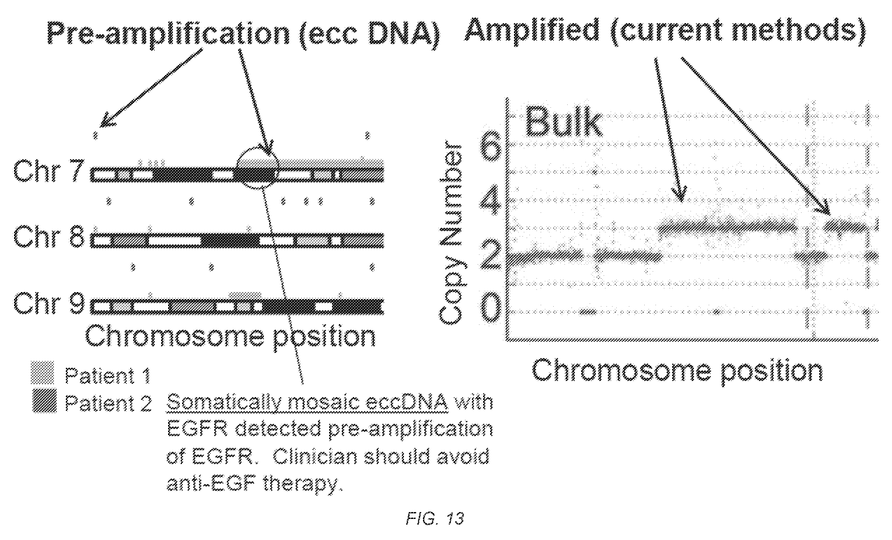

Referring to FIGS. 12 and 13, eccDNAs are detected pre-amplification in Patient 1 (lighter boxes, light blue when shown in color) and in Patient 2 (darker boxes, dark blue when shown in color) to avoid drugs to which the tumor will quickly become resistant by amplifying the drug-resistant gene. Particularly, in the left panel, a karyotype map prepared in accordance with the methods of the presently disclosed subject matter shows that somatically mosaic eccDNA with EGFR are detected to indicate a risk of pre-amplification of EGFR. Thus, a clinician should avoid anti-EGF therapy. In contrast, current methods as shown in the right panel only provide detection after amplification of resistance genes (see Zong et al., Science 2012 Dec 21;338(6114):1622-6).

Thus, the presently disclosed subject matter provides for the detection of drug resistance genes that are poised to amplify. Clinicians should avoid the corresponding drug for the clinical management of cancers, because the resistance gene will quickly amplify and make the cancer resistant to the drug. Thus, in accordance with the methods of the presently disclosed subject matter, single cell ATAC-seq provides for the study of cellular mosaicism of eccDNA in cancers and normal tissues.

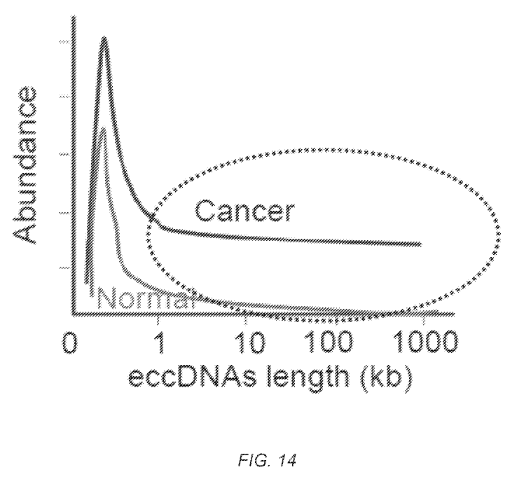

Referring now to FIG. 14, cancers are different from normal cells in having long eccDNA. Also, cell-free eccDNAs are released into blood, including eccDNAs released from cancers into the circulation (Kumar et al., Mol. Cancer Res. 2017, 15:1197-1205; Sin et al., Proc Natl Acad Sci U S A. 2020;117:1658-1665). Thus, circulating cell-free long eccDNA is a biomarker for cancers (liquid biopsy) and eccDNA length can be detected using the presently disclosed method in a convenient and effective manner. Also, eccDNAs released from the fetus into maternal plasma can be used for noninvasive prenatal testing of genetic disease in fetuses. Methods of detecting microDNA in accordance with the presently disclosed subject matter facilitate these applications.

### Example 11

### Exonuclease Method and Kit

In some embodiments, a sample is treated with an exonuclease. Any suitable exonuclease as would be apparent to one of ordinary skill in the art may be used. The following EXAMPLE employs an exonuclease commercially available under the trademark PLASMID-SAFE™.

Isolate DNA from cells.

(a) Trypsinize cells (about 10 million to about 100 million cells) and centrifuge at 200 x g for 5 minutes at 25° C.

Discard the supernatant and wash cell pellet with 10 mL PBS 3 times.

Isolate DNA with HISPEED™ Plasmid Kits (Qiagen #12643)

(b) Purify circular DNA from contaminating linear DNA

Treat the crude DNA fraction (around 1 µg) with PLASMID-SAFE™ exonuclease (Lucigen #E3110K) to digest linear DNA.

Add the following to the reaction:
10x PLASMID-SAFE™ Reaction Buffer 10 µL (final 1x)
25 mM ATP 4 µL (final 1 mM)
10 U/µL PLASMID-SAFE™ ATP-Dependent DNase 2 µL (final 20 units) nuclease-free water to 100 µL

Incubate the reaction at 37° C. for 5 days.

After each 24 hour incubation, add the following to each reaction to continue the enzymatic digestion:
10x PLASMID-SAFE™ 10x Reaction Buffer 0.6 µL
25 mM ATP 4 µL
10 U/µL PLASMID-SAFE™ ATP-Dependent DNase 2 µL
Purify circular DNA with DNA Clean & Concentrator kit (Zymo Research #D4013)

Table 11

Scrip for Read Algorithm
#Arg1 = Number of processors
#Arg2 = Genome or index file “/hdata1/MICRODNA-HG38/hg38.fa”
#Arg3 = fastq file 1 “1E_S1_L1-L4 R1_001.fastq”
#Arg4 = fastq file 2 “1E_S1_L1-L4_R2_001.fastq”
#Arg5 = minNonOverlap between two split reads “10”
#Arg6 = Sample name “IE”
#Arg7 = genome build “hg38”
#Usage: bash “Number of processors” “/path-of-whole-genome-file/hg38.fa” “fastq file 1” “fastq file 2” “minNonOverlap between two split reads” “Sample name” “genome build”
#bash circle_finder-pipeline-bwa-mem-samblaster.sh 16 hg38.fa 1E_S1_L1-L4_Rl_001.fastq.75bp-R1.fastq 1E_S1_L1-L4_R2_001.fastq.75bp-R2.fastq 10 1E hg38

### Step 1: Mapping

bwa mem -t $1 $2 $3 $4 | samblaster -e --minNonOverlap $5 -d $6-$7\.disc.sam -s
$6-$7\.split.sam -u $6-$7\.unmap.sam > $6-$7\.sam

### Step 2: Converting (sam2bam), Sorting and Indexing Mapped Reads. Output of this Step is Input in Step 3

samtools view -@ $1 -bS $6-$7\.sam -o $6-$7\.bam
samtools sort -@ $1 -O bam -o $6-$7\.sorted.bam $6-$7\.bam
samtools index $6-$7\.sorted.bam
samtools view -@ $1 -bS $6-$7\.disc.sam > $6-$7\.disc.bam
samtools view -@ $1 -bS $6-$7\.split.sam > $6-$7\.split.bam
samtools view -@ $1 -bS $6-$7\.unmap.sam > $6-$7\.unmap.bam

### Step 3: Extract Concordant Pairs with Headers

samtools view -@ $1 -hf 0x2 $6-$7\.sorted.bam -bS > $6-$7\.concordant.bam

### Step 4: Converting Bam to Bed Format (Remember Bedtools Generate 0 Based Co-Ordinates)

bedtools bamtobed -cigar -i $6-$7\.split.bam | sed -e s/ _2\V2/\ 2/g | sed -e s/-1\V1/\
⅟g | awk '{printf ("%s\t%d\t%d\t%s\t%d\t%d\t%s\t%s\n",$1,$2,$3,$4,$5,$6,$7,$8)}' |
awk 'BEGIN{FS=OFS="\t"} {gsub("M", " M ", $8)} 1' | awk 'BEGIN{FS=OFS="\t"}
{gsub("S", " S ", $8)} 1' | awk'BEGIN{FS=OFS="\t"} {gsub("H", " H ", $8)} 1' | awk
'BEGIN{FS=OFS=" "} {if (($9=="M" && $NF=="H") || ($9=="M" && $NF=="S"))
{printf ("%s\tfirst\n",$0)} else if (($9=="S" && $NF=="M") || ($9=="H" &&
$NF=="M"))
{printf ("%s\tsecond\n",$0)} }' | awk'BEGIN{FS=OFS="\t"} {gsub("\ ", "", $8)} 1' > $6-
$7\.split.txt
bedtools bamtobed -cigar -i $6-$7\.concordant.bam | sed -e s/\V/\ /g | awk '{printf
("%s\t%d\t%d\t%s\t%d\t%d\t%s\t%s\n",$1,$2,$3,$4,$5,$6,$7,$8)}' > $6-
$7\.concordant.txt
bedtools bamtobed -cigar -i $6-$7\.disc.bam | sed -e s/\V/\ /g | awk '{printf
("%s\t%d\t%d\t%s\t%d\t%d\t%s\t%s\n",$1,$2,$3,$4,$5,$6,$7,$8)}' > $6-$7\.disc.txt

### Step 5: Calculating the Read-ID Frequency. The Frequency of 2 would Indicate that Particular Read is Uniquely Mapping in Genome and there are Only Two Split-Mapped Reads

awk '{print $4}' $6-$7\.split.txt | sort | uniq -c > $6-$7\.split.id-freq.txt
#This file "$6-$7\.split.id-freq.txt" will be used for collecting split id that have
frequency equal to 4.
awk '$1=="2" {print $2}' $6-$7\.split.id-freq.txt > $6-$7\.split.id-freq2.txt
awk '$1=="4" {print $2}' $6-$7\.split.id-freq.txt > $6-$7\.split.id-freq4.txt
awk '{print $4}' $6-$7\.concordant.txt | sort | uniq -c > $6-$7\.concordant.id-freq.txt
#The following command will chose (may not be always true) one concordant and 2
split read
awk '$1=="3" {print $2}' $6-$7\.concordant.id-freq.txt > $6-$7\.concordant.idfreq3.
txt
awk '$1>3 {print $2}' $6-$7\.concordant.id-freq.txt > $6-$7\.concordant.id-freqGr3.txt

### Step 6: Selecting Split Reads that were 1) Mapped Uniquely and 2) Mapped on More than One Loci. For Normal MicroDNA Identification no need to use the “freqGr2” File

grep -w -Ff $6-$7\.split.id-freq2.txt $6-$7\.split.txt > $6-$7\.split_freq2.txt
grep -w -Ff $6-$7\.split.id-freq4.txt $6-$7\.split.txt > $6-$7\.split_freq4.txt

#Selecting concordant pairs that were 1) mapped uniquely and 2) mapped on more than one loci (file “freqGr3.txt”)

grep -w -Ff $6-$7\.concordant.id-freq3.txt $6-$7\.concordant.txt > $6-
$7\.concordant_freq3.txt
grep -w -Ff $6-$7\.concordant.id-freqGr3.txt $6-$7\.concordant.txt > $6-
$7\.concordant_freqGr3.txt

### Step 7: Putting Split Read with Same ID in One Line

sed 'N;s/\n/\t/' $6-$7\.split_freq2.txt > $6-$7\.split_freq2.oneline.txt
sed 'N;s/\n/\t/' $6-$7\.split_freq4.txt > $6-$7\.split_freq4.oneline.txt

### Step 8: Split Reads Map on Same Chromosome and Map on Same Strand

Finally extracting id (split read same chromosome, split read same strand), collecting all the split reads that had quality >0

awk '$1==$10 && $7==$16 && $6>0 && $15>0 {print $4} ' $6-
$7\.split_freq2.oneline.txt > $6-$7\.split_freq2.oneline.S-R-S-CHR-S-ST.ID.txt

### Step 9: Based on Unique ID I am Extracting One Continuously Mapped Reads and Their Partner Mapped as Split Read (3 Lines for Each ID)

grep -w -Ff $6-$7\.split_freq2.oneline.S-R-S-CHR-S-ST.ID.txt $6-
$7\.concordant_freq3.txt > $6-$7\.concordant_freq3.2SPLIT-1M.txt

### Step 10: Sorting Based on Read-ID Followed by Length of Mapped Reads

awk 'BEGIN{FS=OFS="\t"} {gsub("M", " M ", $8)} 1' $6-
$7\.concordant_freq3.2SPLIT-1M.txt | awk'BEGIN{FS=OFS="\t"} {gsub("S", " S ",
$8)} 1' | awk 'BEGIN{FS=OFS="\t"} {gsub("H", " H ", $8)} 1' | awk
'BEGIN{FS=OFS=" "} {if (($9=="M" && $NF=="H") || ($9=="M" && $NF=="S"))
{printf ("%s\tfirst\n",$0)} else if (($9=="S" && $NF=="M") || ($9=="H" &&
$NF=="M"))
{printf ("%s\tsecond\n",$0)} else {printf ("%s\tconfusing\n",$0)}}' | awk
'BEGIN{FS=OFS="\t"} {gsub("\ ", "", $8)} 1' | awk'{printf ("%s\t%d\n",$0,($3-$2)+1)}'
| sort -k4,4 -k10,10n | sed 'N;N;s/\n/\t/g' | awk{if ($5==$15) {print $0} else if
(($5=="1" && $15=="2" && $25=="1") || ($5=="2" && $15=="1" && $25=="2"))
{printf
("%s\t%d\t%d\t%s\t%d\t%d\t%s\t%s\t%s\t%d\t%s\t%d\t%d\t%s\t%d\t%d\t%s\t%s\t%
s\t%d\t%s\t%d\t%d\t%s\t%d\t%d\t%s\t%s\t%s\t%d\n",
$1,$2,$3,$4,$5,$6,$7,$8,$9,$10,$21,$22,$23,$24,$25,$26,$27,$28,$29,$30,$11,$1
2,$13,$14,$15,$16,$17,$18,$19,$20)} else if (($5==" 1" && $15=="2" && $25=="2")
|| ($5=="2" && $15=="1" && $25==" 1")) {printf
("%s\t%d\t%d\t%s\t%d\t%d\t%s\t%s\t%s\t%d\t%s\t%d\t%d\t%s\t%d\t%d\t%s\t%s\t%
s\t%d\t%s\t%d\t%d\t%s\t%d\t%d\t%s\t%s\t%s\t%d\n",
$11,$12,$13,$14,$15,$16,$17,$18,$19,$20,$21,$22,$23,$24,$25,$26,$27,$28,$29,
$30,$1,$2,$3,$4,$5,$6,$7,$8,$9,$10)} }' > $6-$7\.concordant_freq3.2SPLIT-
1M.inoneline.txt

### Step 11: Unique Number of MicroDNA with Number of Split Reads

awk '$1==$11 && $1==$21 && $7==$17' $6-$7\.concordant_freq3.2SPLIT-
1M.inoneline.txt | awk '($7=="+" && $27=="-") || ($7=="-" && $27=="+")' | awk{if
($17=="+" && $19=="second" && $12<$2 && $22>=$12 && $23<=$3) {printf
("%s\t%d\t%d\n",$1,$12,$3)} else if ($7=="+" && $9=="second" && $2<$12 &&
$22>=$2 && $23<=$13) {printf ("%s\t%d\t%d\n",$1,$2,$13)} else if ($17=="-" &&
$19=="second" && $12<$2 && $22>=$12 && $23<=$3) {printf
("%s\t%d\t%d\n",$1,$12,$3)} else if ($7=="-" && $9=="second" && $2<$12 &&
$22>=$2 && $23<=$13) {printf ("%s\t%d\t%d\n",$1,$2,$13)} }' | sort | uniq -c | awk
'{printf ("%s\t%d\t%d\t%d\n",$2,$3,$4,$1)}' > $6-$7\.microDNA-JT.txt
rm *hg38.sam *hg38.bam

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein. Buenrostro et al., Curr Protoc Mol Biol 109, 21 29 21-29 (2015).

Corces et al., Science 362, (2018).

Dillon et al., Cell Rep 11, 1749-1759 (2015).

Kumar et al., Mol Cancer Res 15, 1197-1205 (2017).

Shibata et al., Science 336, 82-86 (2012).

Moller et al., G3 (Bethesda) 6, 453-462 (2015).

Moller et al., Proc Natl Acad Sci USA 112, E3114-3122 (2015).

Moller et al., Nat Commun 9, 1069 (2018).

deCarvalho et al., Nat Genet 50, 708-717 (2018).

Shoura et al., G3 (Bethesda) 7, 3295-3303 (2017).

Turner et al., Nature 543, 122-125 (2017).

Wu et al., Nature 575, 699-703 (2019).

Morton et al., Cell 179, 1330-1341 e1313 (2019).

Xie et al., Cell 175, 1228-1243 e1220 (2018).

Maher et al., Cell 148, 29-32 (2012).

Libermann et al., Nature 313, 144-147 (1985).

Maire et al., Neuro Oncol 16 Suppl 8, viii1-6 (2014).

Xu et al., Acta Neuropathol 137, 123-137 (2019).

Bailey et al., Cell 174, 1034-1035 (2018).

Tutar, Comp Funct Genomics 2012, 424526 (2012).

Corces et al., Nat Methods 14, 959-962 (2017).

Altschul et al. (1990a) J Mol Biol 215:403-410.

Altschul et al. (1990b) Proc Natl Acad Sci USA 87:14:5509-5513.

Altschul et al. (1997) Nucleic Acids Res 25:3389-3402.

Anders et al. (2014) Bioinformatics 31:166-169.

Ausubel et al. (1995) Current Protocols in Molecular Biology, Greene Publishing. Friedman et al. (2010) J Stat Softw 33:1-22.

Gait (1984) Oligonucleotide Synthesis: A Practical Approach, IRL Press, Oxford, England.

Gautier et al. (2004) Bioinformatics 20:307-315.

Glover (1985) DNA Cloning: a Practical Approach. Oxford Press, Oxford.

Gross & Mienhofer (eds.) (1981) The Peptides, Vol. 3. Academic Press, New York, New York, United States of America, pp. 3-88.

Harlow & Lane (1988) Antibodies, a Laboratory Manual, Cold Spring Harbor Laboratory Publications, Cold Spring Harbor, New York, United States of America.

Karlin & Altschul (1990) Proc Natl Acad Sci USA 87:2264-2268.

Karlin & Altschul (1993), Proc Natl Acad Sci USA 90:5873-5877.

Roe et al. (1996) DNA Isolation and Sequencing: Essential Techniques, John Wiley, New York, New York, United States of America.

Sambrook et al. (1989) Molecular Cloning: A Laboratory Manual, Cold Spring Harbor Laboratory Publications, Cold Spring Harbor, New York, United States of America.

Suykens & Vandewalle (1999) Neural Processing Letters 9:293-300.

U.S. Pat. Application Publication Nos. 2010/0120097; 2011/0189679; 2014/0113333; 2015/0307874; 2018/0064695; 2018/0169084; 2019/0030012; 2019/0282565; 2010/0120098, 2016/0060691, 2019/0032128.

U.S. Pat. Nos. 3,974,281; 5,800,992; 6,004,755; 6,013,449; 6,020,135; 6,033,860; 6,040,138; 6,177,248; 6,251,601; 6,309,822; 6,762,180; 7,824,856; 8,592,462; 9,884,802; 9,920,367; 10,028,966; 10,105,365; 10,227,584; each of which is incorporated by reference in its entirety.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method of detecting an extrachromosomal circular DNA (eccDNA) in a biological sample, the method comprising treating the biological sample to produce a tagged linearized fragment of genomic DNA; and determining whether the tagged linearized fragment is derived from an eccDNA to thereby detect the eccDNA.

2. The method of claim 1, wherein treating the biological sample to produce a tagged linearized fragment of genomic DNA comprises treating the biological sample with an insertional enzyme complex to produce a tagged linearized fragment of genomic DNA.

3. The method of claim 1 wherein determining whether the tagged linearized fragment is derived from an eccDNA comprises amplifying the tagged linearized fragment of genomic DNA.

4. The method of claim 1 wherein determining whether the tagged linearized fragment is derived from an eccDNA comprises sequencing the tagged linearized fragment of genomic DNA.

5. The method of claim 1 wherein determining whether the tagged linearized fragment is derived from an eccDNA comprises detecting a junctional sequence in the tagged linearized fragment.

6. The method of claim 5, wherein detecting a junctional sequence comprises employing read pairs.

7. The method of claim 1 further comprising treating the sample with an exonuclease prior to treating the biological sample to produce a tagged linearized fragment of genomic DNA, optionally treating the sample with an exonuclease prior to treating the biological sample with an insertional enzyme complex to produce a tagged linearized fragment enriched from eccDNA.

8. The method of claim 1 wherein the insertional enzyme complex comprises an insertional enzyme and at least two adaptors molecules.

9. The method of claim 2 wherein the insertional enzyme is a transposase.

10. The method of according to claim 9, wherein the transposase is a Tn5 transposase.

11. The method of claim 1 wherein the sample comprises a biopsy or a blood sample.

12. The method of claim 1, wherein the subject is a subject suffering from a cancer or suspected to be suffering from a cancer or a subject having a genetic disease or disorder or suspected to have a genetic disease or disorder,.

13. The method of claim 12, wherein the subject having a genetic disease or disorder or suspected to have a genetic disease or disorder is a fetus and the sample comprises a maternal blood sample.

14. A method, comprising analyzing a sample from a subject using the method of claim 1, to detect eccDNA; and providing a diagnosis or prognosis based on the detected eccDNA.

15. The method of claim 14, wherein providing a diagnosis or prognosis comprises identifying a cell type in the subject, identifying a cell population, identifying a tissue type, and/or identifying a nucleic acid sequence on the eccDNA.

16. The method of claim 14, further comprising choosing a therapy based on the diagnosis or prognosis, optionally based on the identified cell type, cell population, tissue type, or nucleic acid.

17. A method of detecting a cell type, a population of cells, or a tissue type in a subject, the method comprising:
(a) detecting an extrachromosomal circular DNA (eccDNA) in a biological sample from the subject by:
(i) treating the biological sample to produce a tagged linearized fragment of genomic DNA; and
(ii) determining whether the tagged linearized fragment is derived from an eccDNA to thereby detect the eccDNA; and
(b) determining a genomic region from which the eccDNA is derived to thereby detect a cell type, a population of cells or a tissue type in a subject.

18. A method of detecting a nucleic acid sequence associated with a condition in a subject, the method comprising:
(a) detecting an extrachromosomal circular DNA (eccDNA) in a sample from the subject by:
(i) treating the biological sample to produce a tagged linearized fragment, optionally enriched from eccDNA; and
(ii) determining whether the tagged linearized fragment is derived from an eccDNA to thereby detect the eccDNA; and
(b) detecting a presence of a nucleic acid sequence on the eccDNA, wherein the nucleic acid sequence is associated with a condition in the subject.

19. The method of claim 17, wherein treating the biological sample to produce a tagged linearized fragment, optionally enriched from genomic eccDNA, comprises treating the biological sample with an insertional enzyme complex to produce a tagged linearized fragment of genomic DNA, optionally treating the biological sample with an exonuclease to digest genomic linear DNA and then with an insertional enzyme complex to produce a tagged linearized fragment of genomic DNA.

20. The method of claim 17, wherein determining whether the tagged linearized fragment is derived from an eccDNA comprises amplifying the tagged linearized fragment of genomic DNA.

21. The method of claim 17, wherein determining whether the tagged linearized fragment is derived from an eccDNA comprises sequencing the tagged linearized fragment of genomic DNA.

22. The method of claim 17, wherein determining whether the tagged linearized fragment is derived from an eccDNA comprises detecting a junctional sequence in the tagged linearized fragment.

23. The method of claim 22, wherein detecting a junctional sequence comprises employing read pairs.

24. The method of claim 17, further comprising treating the sample with an exonuclease prior to treating the biological sample to produce a tagged linearized fragment of genomic DNA.

25. The method of claim 19, wherein the insertional enzyme complex comprises an insertional enzyme and at least two adaptors molecules.

26. The method of claim 19, wherein the insertional enzyme is a transposase.

27. The method of according to claim 26, wherein the transposase is a Tn5 transposase.

28. The method of claim 17, wherein the sample comprises a biopsy or a blood sample.

29. The method of claim 17, wherein the subject is a subject suffering from a cancer or suspected to be suffering from a cancer or a subject having a genetic disease or disorder or suspected to have a genetic disease or disorder.

30. The method of claim 29, wherein the subject having a genetic disease or disorder or suspected to have a genetic disease or disorder is a fetus and the sample comprises a maternal blood sample.

31. The method of claim 17, wherein identifying a cell type in the subject, identifying a cell population, identifying a tissue type; and/or identifying a nucleic acid sequence on the eccDNA further comprises identifying a cancer or a genetic disease or disorder in the subject.

32. The method of claim 17, further comprising choosing a therapy based on the identified cell type, cell population, tissue type, and/or nucleic acid sequence.

33. A kit for detecting eccDNA in a sample, wherein the kit comprises one or more reagents suitable for carrying out the method according to claim 1, and instructional material for employing the one or more reagents.
